(19) United States
(12) Patent Application Publication
(10) Pub. No.: US 2025/0061991 A1
(43) Pub. Date: Feb. 20, 2025

(54) SYSTEMS AND METHODS FOR DEVELOPING AND UTILIZING PARTICIPANT-ADJUSTED ENDPOINTS

(71) Applicant: Folia Health, Inc., Boston, MA (US)

(72) Inventors: Nell Meosky Luo, Somerville, MA (US); Dan Toffling, Wellesley, MA (US); Luke Stephens, Cambridge, MA (US)

(21) Appl. No.: 18/807,654

(22) Filed: Aug. 16, 2024

Related U.S. Application Data
(60) Provisional application No. 63/520,128, filed on Aug. 17, 2023.

(51) Int. Cl. G16H 10/60; G06Q 10/109

(52) U.S. Cl. CPC G16H 10/60; G06Q 10/109

(57) ABSTRACT

A healthcare management computing system including a processor in communication with a memory is described. The processor is configured to receive a first input from a user computing device and identify historical health data stored in the memory. The processor is also configured to, based upon health data and the historical health data, determine additional health data associated with the health data and cause a plurality of data fields associated with the user profile to be populated with the additional health data. The processor is further configured to receive a second input from the user computing device, the second input including modified additional health data, and update the historical health data in the memory to include the modified additional health data as being associated with the health data.

### CROSS REFERENCE TO RELATED APPLICATIONS

This application claims the benefit of priority to U.S. Provisional Patent Application No. 63/520,128, filed Aug. 17, 2023, which is hereby incorporated by reference herein in its entirety.

### TECHNICAL FIELD

This application is directed to data analysis and, more specifically, to developing and utilizing participant-adjusted endpoints in data analysis.

### BACKGROUND

Health is individual; no two people, even within the same condition category, have an identical experience or manifestation of disease. This phenomenon may be referred to as heterogeneity (e.g., the degree to which the community of individuals experiencing a condition exhibits differences in the primary outcomes-of-interest that describe each individual's experience of the condition and how they assess the impact of a therapy). Across many condition communities, heterogeneity is present. In other words, each individual notices a unique combination of most-important symptoms, and has a particular experience of the impact of a new therapy on his or her disease and quality-of-life.

This phenomenon has been clearly illustrated by COVID-19. There are myriad individual responses to infection, informed by each person's baseline state of health and a constellation of circumstances. Together, these determine whether a patient loses their sense of smell, develops a fever, or has trouble breathing. Every person infected with COVID-19 has the same starting diagnosis—but the experience of the illness is highly variable (e.g., from asymptomatic to multiorgan failure).

While the degree of variation in the individual experience of COVID-19 is extreme, widespread heterogeneity is present in the manifestation of most chronic conditions, and the way in which new therapies for these conditions impact the health of patients who receive them. For example, in chronic obstructive pulmonary disease (COPD), heterogeneity is found in clinical manifestations, described physiology, imaging findings, and inflammatory reactions exhibited by individuals. Thus, individualized treatment is optimal.

As another example, in multiple sclerosis (MS), a neurological chronic condition found in a much younger cohort than COPD, there is also widespread variation in phenotype, with only a few clinical features of the disease shared by most or all people living with MS. Even within the three major sub-groups of MS that have been identified, there persists high heterogeneity; outcomes in relapsing-remitting MS vary from minimal disability accumulating over 20+ years, to rapidly progressive cases in which significant disability is seen over only a few years (e.g., there is no reliable single pathway treatment).

This phenomenon exists even in well-characterized conditions advantaged by relatively straightforward diagnostics, and seemingly more homogenous populations. In cystic fibrosis (CF), a rare genetic condition that affects cystic fibrosis transmembrane conductance regulator (CFTR) expression resulting in respiratory, digestive, and other complications, there is a relatively clear diagnostic pathway, and a well-understood set of primary genotypes and phenotypes. However, there remains variation at the individual level in the experience of the condition. People living with CF and their caregivers experience a broad range of symptoms and must maintain complex treatment plans, which in turn can contribute to complications of the disease.

In order to accurately measure outcomes experienced in the course of living with a disease, and to establish a more robust understanding of the impact of new therapies, it is imperative that individual variation is accounted for. Failure to capture the unique experience of each individual can result in missing important signals regarding the efficacy of a new therapy and the current state of an individual's health. Accordingly, improved systems and methods for developing and utilizing participant-adjusted endpoints in data analysis are desired.

### BRIEF DESCRIPTION

In one aspect, a healthcare management computing system including at least one processor in communication with at least one memory is provided. The at least one processor is configured to receive a first input from a user computing device, the first input including health data associated with a first time period and a user profile associated with the user computing device, and identify historical health data stored in the at least one memory and associated with the health data wherein the historical health data includes first historical health data associated with the user profile before the first time period and second historical health data associated with a plurality of user profiles different from the user profile before the first time period. The at least one processor is also configured to, based upon the health data and the historical health data, determine additional health data associated with the health data and cause a plurality of data fields associated with the user profile to be populated with the additional health data. The at least one processor is further configured to receive a second input from the user computing device, the second input including modified additional health data including at least one modification to the additional health data, and update the historical health data in the at least one memory to include the modified additional health data as being associated with the health data.

In another aspect, at least one non-transitory computer-readable storage medium with instructions stored thereon is described. The instructions, in response to execution by at least one processor, cause the at least one processor to receive a first input from a user computing device, the first input including health data associated with a first time period and a user profile associated with the user computing device, and identify historical health data stored in the at least one non-transitory computer-readable storage medium and associated with the health data wherein the historical health data includes first historical health data associated with the user profile before the first time period and second historical health data associated with a plurality of user profiles different from the user profile before the first time period. The instructions also cause the at least one processor to, based upon the health data and the historical health data, determine additional health data associated with the health data, and cause a plurality of data fields associated with the user profile to be populated with the additional health data. The instructions further cause the at least one processor to receive a second input from the user computing device, the second input including modified additional health data including at least one modification to the additional health data, and update the historical health data in the at least one non-transitory computer-readable storage medium to include the modified additional health data as being associated with the health data.

In another aspect, a method for healthcare management implemented by at least one processor in communication with at least one memory is described. The method includes receiving a first input from a user computing device, the first input including health data associated with a first time period and a user profile associated with the user computing device, and identifying historical health data stored in the at least one memory and associated with the health data wherein the historical health data includes first historical health data associated with the user profile before the first time period and second historical health data associated with a plurality of user profiles different from the user profile before the first time period. The method also includes, based upon the health data and the historical health data, determining additional health data associated with the health data and causing a plurality of data fields associated with the user profile to be populated with the additional health data. The method further includes receiving a second input from the user computing device, the second input including modified additional health data including at least one modification to the additional health data, and updating the historical health data in the at least one memory to include the modified additional health data as being associated with the health data.

### BRIEF DESCRIPTION OF THE DRAWINGS

FIG. 1 illustrates an example participant-adjusted endpoint (PAEP) system, as described herein.

FIG. 2 illustrates another example of the PAEP system described herein.

FIG. 3 illustrates an example process for developing and utilizing participant-adjusted endpoints as implemented by the PAEP system shown in FIG. 1.

FIG. 4 illustrates an example configuration of a computing device, in accordance with the present disclosure.

FIG. 5 illustrates an example configuration of server components for developing and utilizing participant-adjusted endpoints, in accordance with the present disclosure.

FIG. 6 illustrates an example information capture process implemented by the PAEP system shown in FIG. 1.

FIG. 7 illustrates an process for building and scheduling items as implemented by the PAEP system shown in FIG. 1.

FIG. 8 illustrates an example dashboard generated by the PAEP system shown in FIG. 1.

FIG. 9 illustrates another example method for developing and utilizing participant-adjusted endpoints as implemented by the PAEP system shown in FIG. 1.

FIG. 10 illustrates an example commonality and frequency graph illustrating information utilized by the PAEP system shown in FIG. 1.

FIG. 11 illustrates an example registration and setup page utilized by the PAEP system shown in FIG. 1.

FIG. 12 illustrates another example registration and setup page utilized by the PAEP system as shown in FIG. 11.

FIG. 13 illustrates another example registration and setup page utilized by the PAEP system as shown in FIG. 11.

FIG. 14 illustrates another example registration and setup page utilized by the PAEP system as shown in FIG. 11.

FIG. 15 illustrates another example registration and setup page utilized by the PAEP system as shown in FIG. 11.

FIG. 16 illustrates another example registration and setup page utilized by the PAEP system as shown in FIG. 11.

FIG. 17 illustrates another example registration and setup page utilized by the PAEP system as shown in FIG. 11.

FIG. 18 illustrates another example registration and setup page utilized by the PAEP system as shown in FIG. 11.

FIG. 19 illustrates another example registration and setup page utilized by the PAEP system as shown in FIG. 11.

FIG. 20 illustrates another example registration and setup page utilized by the PAEP system as shown in FIG. 11.

FIG. 21 illustrates another example registration and setup page utilized by the PAEP system as shown in FIG. 11.

FIG. 22 illustrates another example registration and setup page utilized by the PAEP system as shown in FIG. 11.

FIG. 23 illustrates an example dashboard GUI utilized by the PAEP system.

FIG. 24 illustrates an example GUI provided in response to selection of the questions selector utilized by the PAEP system.

FIG. 25 illustrates a next GUI after the user selected track in

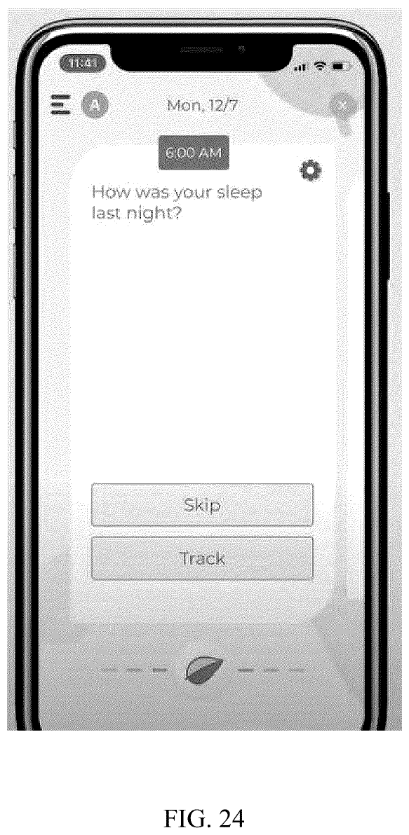

FIG. 24.

FIG. 26 illustrates another next GUI after the user selected track in FIG. 24.

FIG. 27 illustrates another next GUI after the user selected track in FIG. 24.

FIG. 28 illustrates another next GUI after the user selected track in FIG. 24.

FIG. 29 illustrates another next GUI after the user selected track in FIG. 24.

FIG. 30 illustrates another next GUI after the user selected track in FIG. 24.

FIG. 31 illustrates another next GUI after the user selected track in FIG. 24.

FIG. 32 illustrates another next GUI after the user selected track in FIG. 24.

FIG. 33 illustrates another next GUI after the user selected track in FIG. 24.

FIG. 34 illustrates another next GUI after the user selected track in FIG. 24.

FIG. 35 illustrates another next GUI after the user selected track in FIG. 24.

FIG. 36 illustrates another next GUI after the user selected track in FIG. 24.

FIG. 37 illustrates another next GUI after the user selected track in FIG. 24.

FIG. 38 illustrates another next GUI after the user selected track in FIG. 24.

FIG. 39 illustrates another next GUI after the user selected track in FIG. 24.

FIG. 40 illustrates another next GUI after the user selected track in FIG. 24.

FIG. 41 illustrates another next GUI after the user selected track in FIG. 24.

FIG. 42 illustrates another next GUI after the user selected track in FIG. 24.

FIG. 43 illustrates another next GUI after the user selected track in FIG. 24.

FIG. 44 illustrates another next GUI after the user selected track in FIG. 24 showing the completion of daily tracking.

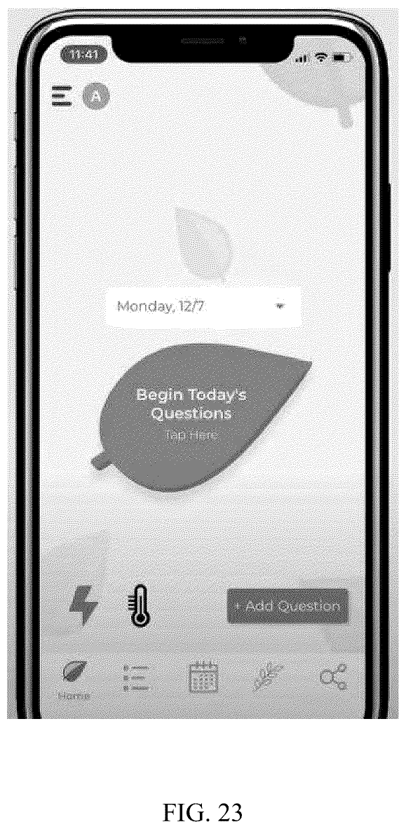

FIG. 45 illustrates an example GUI shown in response to user selection of an insights tab shown on the dashboard of FIG. 23.

FIG. 46 illustrates a GUI shown in response to selection of the new graph selector shown in FIG. 45.

FIG. 47 illustrates another GUI shown following selection of the new graph selector shown in FIG. 45.

FIG. 48 illustrates the GUI of FIG. 47 upon user entry of joint into the search bar.

FIG. 49 illustrates the GUI of FIG. 46, now including joint pain (scale of 1-10), as selected in FIG. 48.

FIG. 50 illustrates the GUI of FIG. 49, where the user has indicated they would like an additional item to be shown in the requested graph.

FIG. 51 illustrates another GUI shown following selection of the new graph selector shown in FIG. 45.

FIG. 52 illustrates another GUI shown following selection of the new graph selector shown in FIG. 45.

FIG. 53 illustrates a requested graph GUI as generated following FIGS. 45-52.

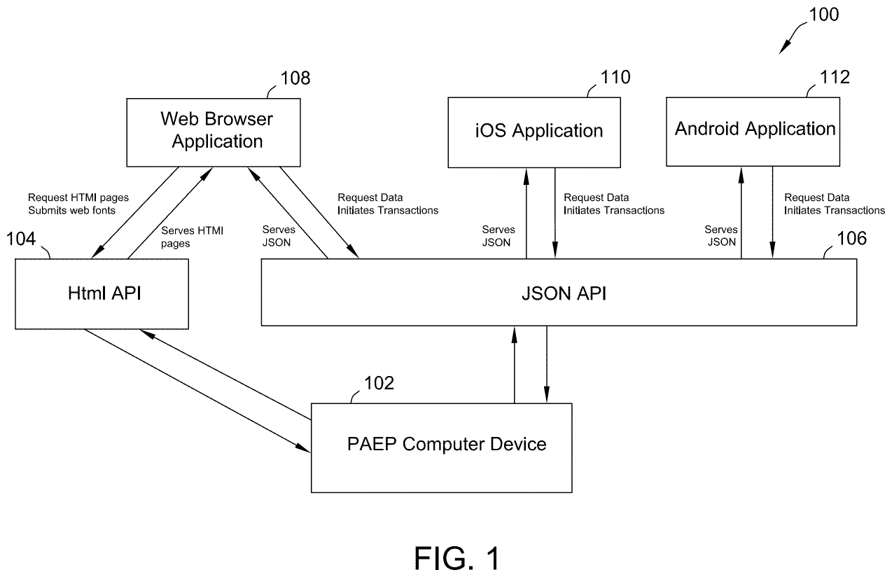

FIG. 54 illustrates an example home screen generated by the PAEP system shown in FIG. 1.

FIG. 55 illustrates an example screenshot and/or interface for developing and utilizing participant-adjusted endpoints as implemented by the PAEP system shown in FIG. 1.

FIG. 56 illustrates an example screenshot and/or interface for developing and utilizing participant-adjusted endpoints as implemented by the PAEP system shown in FIG. 1.

FIG. 57 illustrates an example screenshot and/or interface for treatment tracking as implemented by the PAEP system shown in FIG. 1.

FIG. 58 illustrates an example screenshot and/or interface for treatment tracking as implemented by the PAEP system shown in FIG. 1.

FIG. 59 illustrates an example screenshot and/or interface for heat map visualization as implemented by the PAEP system shown in FIG. 1.

FIG. 60 illustrates an example screenshot and/or interface for flare tracking as implemented by the PAEP system shown in FIG. 1.

FIG. 61 illustrates an example screenshot and/or interface for flare tracking as implemented by the PAEP system shown in FIG. 1.

FIG. 62 illustrates an example screenshot and/or interface of a baseline function for home-reported outcomes as implemented by the PAEP system shown in FIG. 1.

FIG. 63 illustrates another example screenshot and/or interface of the baseline function for home-reported outcomes as shown in FIG. 62.

FIG. 64 illustrates another example screenshot and/or interface of the baseline function for home-reported outcomes as shown in FIG. 62.

FIG. 65 illustrates another example screenshot and/or interface of the baseline function for home-reported outcomes as shown in FIG. 62.

FIG. 66 illustrates another example screenshot and/or interface of the baseline function for home-reported outcomes as shown in FIG. 62.

FIG. 67 illustrates another example screenshot and/or interface of the baseline function for home-reported outcomes as shown in FIG. 62.

FIG. 68 illustrates another example screenshot and/or interface of a tracking function for home-reported outcomes implemented by the PAEP system shown in FIG. 1.

FIG. 69 illustrates another example screenshot and/or interface of the tracking function for home-reported outcomes as shown in FIG. 68.

FIG. 70 illustrates another example screenshot and/or interface of the tracking function for home-reported outcomes as shown in FIG. 68.

FIG. 71 illustrates another example screenshot and/or interface of the tracking function for home-reported outcomes as shown in FIG. 68.

FIG. 72 illustrates another example screenshot and/or interface of the tracking function for home-reported outcomes as shown in FIG. 68.

FIG. 73 illustrates another example screenshot and/or interface of the tracking function for home-reported outcomes as shown in FIG. 68.

FIG. 74 illustrates another example screenshot and/or interface of the tracking function for home-reported outcomes as shown in FIG. 68.

FIG. 75 illustrates another example screenshot and/or interface of the tracking function for home-reported outcomes as shown in FIG. 68.

FIG. 76 illustrates another example screenshot and/or interface of the tracking function for home-reported outcomes as shown in FIG. 68.

FIG. 77 illustrates an example screenshot and/or interface of a treatment group function for home-reported outcomes implemented by the PAEP system shown in FIG. 1.

FIG. 78 illustrates an example screenshot and/or interface of the treatment group function for home-reported outcomes as shown in FIG. 77.

FIG. 79 illustrates an example screenshot and/or interface of the treatment group function for home-reported outcomes as shown in FIG. 77.

FIG. 80 illustrates another example screenshot and/or interface of the baseline function for home-reported outcomes as shown in FIG. 62.

FIG. 81 illustrates another example screenshot and/or interface of the baseline function for home-reported outcomes as shown in FIG. 62.

FIG. 82 illustrates an example screenshot and/or interface of a copy function for home-reported outcomes as implemented by the PAEP system shown in FIG. 1.

FIG. 83 illustrates an example screenshot and/or interface of the copy function for home-reported outcomes as shown in FIG. 82.

FIG. 84 illustrates an example screenshot and/or interface of the copy function for home-reported outcomes as shown in FIG. 82.

FIG. 85 illustrates an example screenshot and/or interface of the copy function for home-reported outcomes as shown in FIG. 82.

FIG. 86 illustrates an example screenshot and/or interface of a suggestion function for home-reported outcomes as implemented by the PAEP system shown in FIG. 1.

FIG. 87 illustrates an example screenshot and/or interface of the suggestion function for home-reported outcomes as shown in FIG. 86.

FIG. 88 illustrates an example screenshot and/or interface of tag functions for home-reported outcomes as implemented by the PAEP system shown in FIG. 1.

FIG. 89 illustrates an example screenshot and/or interface of tag functions for home-reported outcomes as seen in FIG. 88.

FIG. 90 illustrates an example screenshot and/or interface of a flare tracking function implemented by the PAEP system shown in FIG. 1.

FIG. 91 illustrates another example screenshot and/or interface of the flare tracking function as shown in FIG. 90.

FIG. 92 illustrates another example screenshot and/or interface of the flare tracking function as shown in FIG. 90.

FIG. 93 illustrates another example screenshot and/or interface of the flare tracking function as shown in FIG. 90.

FIG. 94 illustrates another example screenshot and/or interface of the flare tracking function as shown in FIG. 90.

FIG. 95 illustrates another example screenshot and/or interface of the flare tracking function as shown in FIG. 90.

FIG. 96A illustrates another example screenshot and/or interface of the flare tracking function as shown in FIG. 90.

FIG. 96B illustrates another example screenshot and/or interface of the flare tracking function as shown in FIG. 90.

FIG. 97 illustrates another example screenshot and/or interface of the flare tracking function as shown in FIG. 90.

FIG. 98 illustrates another example screenshot and/or interface of the flare tracking function as shown in FIG. 90.

FIG. 99 illustrates another example screenshot and/or interface of the flare tracking function as shown in FIG. 90.

FIG. 100 illustrates another example screenshot and/or interface of the flare tracking function as shown in FIG. 90.

FIG. 101 illustrates another example screenshot and/or interface of the flare tracking function as shown in FIG. 90.

FIG. 102 illustrates another example screenshot and/or interface of the flare tracking function as shown in FIG. 90.

FIG. 103 illustrates an example screenshot and/or interface of a research function as implemented by the PAEP system shown in FIG. 1.

FIG. 104 illustrates another example screenshot and/or interface of the research function as shown in FIG. 103.

FIG. 105 illustrates another example screenshot and/or interface of the research function as shown in FIG. 103.

FIG. 106 illustrates another example screenshot and/or interface of the research function as shown in FIG. 103.

FIG. 107 illustrates another example screenshot and/or interface of the research function as shown in FIG. 103.

FIG. 108 illustrates an example screenshot and/or interface of a chapter function implemented by the PAEP system shown in FIG. 1.

FIG. 109 illustrates another example screenshot and/or interface of the chapter function as shown in FIG. 108.

FIG. 110 illustrates another example screenshot and/or interface of the chapter function as shown in FIG. 108.

FIG. 111 illustrates another example screenshot and/or interface of the chapter function as shown in FIG. 108.

FIG. 112 illustrates another example screenshot and/or interface of the chapter function as shown in FIG. 108.

FIG. 113 illustrates another example screenshot and/or interface of the chapter function as shown in FIG. 108.

FIG. 114 illustrates an example screenshot and/or interface of the date selection, trends, and visualization function as implemented by the PAEP system shown in FIG. 1.

FIG. 115 illustrates another example screenshot and/or interface of the date selection, trends, and visualization function as shown in FIG. 114.

FIG. 116 illustrates another example screenshot and/or interface of the date selection, trends, and visualization function as shown in FIG. 114.

FIG. 117 illustrates an example screenshot and/or interface of the journal and search filter functions as implemented by the PAEP system shown in FIG. 1.

FIG. 118 illustrates another example screenshot and/or interface of the journal and search filter functions as shown in FIG. 117.

FIG. 119 illustrates another example screenshot and/or interface of the journal and search filter functions shown in FIG. 117.

FIG. 120 illustrates another example screenshot and/or interface of the journal and search filter functions shown in FIG. 117.

FIG. 121 illustrates another example screenshot and/or interface of the journal and search filter functions shown in FIG. 117.

FIG. 122 illustrates another example screenshot and/or interface of the journal and search filter functions shown in FIG. 117.

FIG. 123 illustrates another example screenshot and/or interface of the journal and search filter functions shown in FIG. 117.

FIG. 124 illustrates another example screenshot and/or interface of the journal and search filter functions shown in FIG. 117.

FIG. 125 illustrates another example screenshot and/or interface of the journal and search filter functions shown in FIG. 117.

FIG. 126A illustrates another example screenshot and/or interface of the journal and search filter functions shown in FIG. 117.

FIG. 126B illustrates another example screenshot and/or interface of the journal and search filter functions shown in FIG. 117.

FIG. 127 illustrates an example method for healthcare management, in accordance with the present disclosure.

FIG. 128 illustrates a block diagram of an example data taxonomy, in accordance with the present disclosure.

FIG. 129 illustrates a block diagram of an example data taxonomy structure for observations, in accordance with the present disclosure.

FIG. 130 illustrates another block diagram of an example data taxonomy as shown in FIG. 129.

FIG. 131 illustrates a flowchart showing an iterative process for updating a data taxonomy, in accordance with the present disclosure.

### DETAILED DESCRIPTION

Described herein are systems and methods for developing and utilizing participant-adjusted endpoints. For example, participant-adjusted endpoints (PAEPs) as used herein may mean an outcome or event used to objectively measure the effect of certain habits and/or treatments at the participant level (e.g., participant-adjusted). To better address individual variation in disease manifestation and to more comprehensively describe the impact of therapy, PAEPs are generated and utilized in a framework that accounts for individual variation by enabling measurement of different outcomes-of-interest among study participants, with population analysis focused on comparison of change at the domain level, and overall degree of change in disease burden experienced by participants.

PAEPs enable a more complete and patient-centered view of individual outcomes and therapeutic impact by, as examples, allowing for the measurement of impact across the full array of individual disease expression, including both outcomes that are commonly experienced in a population and outcomes that are specific to a smaller cluster of individuals, and complementing the clinical perspective with direct patient and caregiver perspectives on which outcomes are most important.

PAEPs are especially useful for application to conditions in which there is heterogeneity of disease expression or experience. They are also helpful in conditions with complex treatment plans, as individuals may have variable responses to the full set of therapies used, and the side-effects experienced as a result of their treatment plans.

For instance, rare disease communities are likely beneficiaries of the PAEP approach, due to the nascent state of endpoint development in many of these conditions, the particularly patient-driven nature of research in these communities, and the phenotype complexity present in many rare diseases. However, there are many communities outside of rare disease in which existing measures do not fully reflect the experience of the condition or the impact of introduction of therapy, and in these situations, the use of PAEPs should be considered.

One embodiment includes a healthcare management computing system including at least one processor in communication with at least one memory, wherein the at least one processor is configured to: receive a first input from a user computing device, the first input including health data associated with a first time period and a user profile associated with the user computing device; identify historical health data stored in the at least one memory and associated with the health data, wherein the historical health data includes first historical health data associated with the user profile before the first time period and second historical health data associated with a plurality of user profiles different from the user profile before the first time period; based upon the health data and the historical health data, determine additional health data associated with the health data; cause a plurality of data fields associated with the user profile to be populated with the additional health data; receive a second input from the user computing device, the second input including modified additional health data including at least one modification to the additional health data; and update the historical health data in the at least one memory to include the modified additional health data as being associated with the health data.

In some embodiments, the at least one processor is further configured to: receive a third input from the user computing device, the third input including second health data associated with a second time period and the user profile, wherein the second health data includes at least one data entry that is the same as the health data; identify the updated historical health data in the at least one memory; and cause a second plurality of data fields associated with the user profile to be populated with the modified additional health data.

In some embodiments, the first time period is one day.

In some embodiments, the health data is associated with one of a typical day for a user associated with the user profile or an atypical day for the user.

In some embodiments, the health data includes at least one of an observation severity, a count, a presence, a numeric value with a unit, a tag, a timing, a duration, a free text note, a voice note, or a media image.

In some embodiments, the at least one processor is further configured to: generate a calendar associated with the user profile; and cause an icon to be added to a portion of the calendar associated with the first time period, wherein the icon is associated with the health data.

In some embodiments, the user profile and the plurality of user profiles different from the user profile are associated with a same health condition.

In some embodiments, the at least one processor is further configured to, based upon the historical health data and the modified additional health data, determine one or more suggestions for a user associated with the user profile to address a health condition identified in the modified additional health data.

One embodiment includes at least one non-transitory computer-readable storage medium with instructions stored thereon that, in response to execution by at least one processor, cause the at least one processor to: receive a first input from a user computing device, the first input including health data associated with a first time period and a user profile associated with the user computing device; identify historical health data stored in the at least one non-transitory computer-readable storage medium and associated with the health data, wherein the historical health data includes first historical health data associated with the user profile before the first time period and second historical health data associated with a plurality of user profiles different from the user profile before the first time period; based upon the health data and the historical health data, determine additional health data associated with the health data; cause a plurality of data fields associated with the user profile to be populated with the additional health data; receive a second input from the user computing device, the second input including modified additional health data including at least one modification to the additional health data; and update the historical health data in the at least one non-transitory computer-readable storage medium to include the modified additional health data as being associated with the health data.

In some embodiments, the at least one processor is further configured to: receive a third input from the user computing device, the third input including second health data associated with a second time period and the user profile, wherein the second health data includes at least one data entry that is the same as the health data; identify the updated historical health data in the at least one non-transitory computer-readable storage medium; and cause a second plurality of data fields associated with the user profile to be populated with the modified additional health data.

In some embodiments, the first time period is one day.

In some embodiments the health data is associated with one of a typical day for a user associated with the user profile or an atypical day for the user.

In some embodiments the health data includes at least one of an observation severity, a count, a presence, a numeric value with a unit, a tag, a timing, a duration, a free text note, a voice note, or a media image.

In some embodiments, the at least one processor is further configured to: generate a calendar associated with the user profile; and cause an icon to be added to a portion of the calendar associated with the first time period, wherein the icon is associated with the health data.

In some embodiments, the user profile and the plurality of user profiles different from the user profile are associated with a same health condition.

In some embodiments, the at least one processor is further configured to, based upon the historical health data and the modified additional health data, determine one or more suggestions for a user associated with the user profile to address a health condition identified in the modified additional health data.

In one embodiment, a method for healthcare management implemented by at least one processor in communication with at least one memory is provided. The method includes: receiving a first input from a user computing device, the first input including health data associated with a first time period and a user profile associated with the user computing device; identifying historical health data stored in the at least one memory and associated with the health data, wherein the historical health data includes first historical health data associated with the user profile before the first time period and second historical health data associated with a plurality of user profiles different from the user profile before the first time period; based upon the health data and the historical health data, determining additional health data associated with the health data; causing a plurality of data fields associated with the user profile to be populated with the additional health data; receiving a second input from the user computing device, the second input including modified additional health data including at least one modification to the additional health data; and updating the historical health data in the at least one memory to include the modified additional health data as being associated with the health data.

In some embodiments, the method includes receiving a third input from the user computing device, the third input including second health data associated with a second time period and the user profile, wherein the second health data includes at least one data entry that is the same as the health data; identifying the updated historical health data in the at least one memory; and causing a second plurality of data fields associated with the user profile to be populated with the modified additional health data.

In some embodiments, the method includes generating a calendar associated with the user profile; and causing an icon to be added to a portion of the calendar associated with the first time period, wherein the icon is associated with the health data.

In some embodiments, the method includes, based upon the historical health data and the modified additional health data, determining one or more suggestions for a user associated with the user profile to address a health condition identified in the modified additional health data.

In one embodiment, a computer program is provided, and the computer program is embodied on a computer-readable medium. In an example embodiment, the system is executed on a single computer system, without requiring a connection to a server computer. In a further example embodiment, the system is run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Apple Inc. located in Cupertino, CA). In yet a further embodiment, the system is run on a Mac OS® environment (Mac OS is a registered trademark of Apple Inc. located in Cupertino, CA). In a further embodiment, the system is run on a Linux® environment (Linux is a registered trademark of Linus Torvalds in the United States and other countries). The system is flexible and designed to run in various different environments without compromising any major functionality. In some embodiments, the system includes multiple components distributed among a plurality of computing devices. One or more components are in the form of computer-executable instructions embodied in a computer-readable medium. The systems and processes are not limited to the specific embodiments described herein. In addition, components of each system and each process can be practiced independently and separately from other components and processes described herein. Each component and process can also be used in combination with other assembly packages and processes.

In one embodiment, a computer program is provided, and the program is embodied on a computer-readable medium and utilizes a Structured Query Language (SQL) with a client user interface front-end for administration and a web interface for standard user input and reports. In another embodiment, the system is web enabled and is run on a business entity intranet. In yet another embodiment, the system is fully accessed by individuals having an authorized access outside the firewall of the business-entity through the Internet. The application is flexible and designed to run in various different environments without compromising any major functionality.

As used herein, an element or step recited in the singular and preceded with the word “a” or “an” should be understood as not excluding plural elements or steps, unless such exclusion is explicitly recited. For example, reference to “a device” may refer to a plurality of devices. Further, references to “example embodiment” or “one embodiment” of the present disclosure are not intended to be interpreted as excluding the existence of additional embodiments that also incorporate the recited features.

As used herein, the term “database” may refer to either a body of data, a relational database management system (RDBMS), or to both. A database may include any collection of data including hierarchical databases, relational databases, flat file databases, object-relational databases, object-oriented databases, and any other structured collection of records or data that is stored in a computer system. The above examples are for example only, and thus, are not intended to limit in any way the definition and/or meaning of the term database. Examples of RDBMS's include, but are not limited to including, Oracle® Database, MySQL, IBM® DB2, Microsoft® SQL Server, Sybase®, and PostgreSQL. However, any database implementation (e.g., relational, document-based) may be used that enables the system and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California).

The term processor, as used herein, may refer to central processing units, microprocessors, microcontrollers, reduced instruction set circuits (RISC), application specific integrated circuits (ASIC), logic circuits, and any other circuit or processor capable of executing the functions described herein.

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are for example only, and are thus not limiting as to the types of memory usable for storage of a computer program.

FIG. 1 illustrates an example participant-adjusted endpoint (PAEP) system 100. As shown in FIG. 1, system 100 includes a PAEP computer device 102 (e.g., including a processor and a database), a hypertext markup language (HTML) application programming interface (API) 104, a JavaScript object notation (JSON) API 106, a first user device 108 (e.g., a personal computer), a second user device 110 (e.g., a mobile device such as a smartphone), and a third user device 112 (e.g., another mobile device such as a smartphone). In some embodiments, API 104 and/or API 106 are implemented on PAEP computer device 102.

In the example embodiment, PAEP computer device 102 is configured to develop and utilize participant-adjusted endpoints, as described herein. In order to implement the systems and methods described herein, PAEP computer device 102 transmits data to and receives data from, as examples device 108 (e.g., via API 104 and/or API 106), device 110 (e.g., via API 106), and/or device 112 (e.g., via API 106). For instance, a web browser application executed on device 108 may request HTML pages and/or submit web forms to API 104 and receive HTML pages from API 104. Further, the web browser application executed on device 108 may request data from and initiate transactions in messages to API 106 and receive appropriate data back in messages from API 106.

As other examples, device 110 (e.g., an iOS application at device 110) may request data and initiate transactions in messages to API 106 and receive appropriate data back in messages from API 106. Further, device 112 (e.g., an Android application at device 112) may request data and initiate transactions in messages to API 106 and receive appropriate data back in messages from API 106.

For example, a participant (e.g., a user) at any of devices 108-112 (e.g., or other computer devices) may desire more accurate tracking of their current care experience and more clarity on what's happening to feel more in control of their care. Accordingly, via system 100 (e.g., any of devices 108-112 in communication with device 102 via API 104 and/or API 106), the user may sign up and onboard to understand the benefits provided by system 100 and device 102. The user may i) set up/update treatments they are using to receive reminders from device 102 to complete them; ii) Set up/update indicators they are watching on an outside data stream; iii) Receive real-time or weekly reminders to track; iv) Track their own observations; v) Pull in outside data; vi) See their health trends and how they're changing; vii) Share their trends and how they're changing; viii) Contribute their insights to their community's research needs; ix) Compare themself to others and to current knowledge to figure out what to do; x) Keep track of what they've learned; and/or xi) Take action and update the plan.

As further examples, a participant (e.g., a user) at any of devices 108-112 (e.g., or other computer devices) may desire to stay on top of living with a complex condition, while making sure they're not missing any major important changes (e.g., wanting to compartmentalize being a patient, but still wanting to be responsible). Accordingly, via system 100 (e.g., any of devices 108-112 in communication with device 102 via API 104 and/or API 106), the user may sign up and onboard to understand the benefits provided by system 100 and device 102. For example, the user may i) Set up/update their treatment plan to have a clear record of what they're using, and to remember refills and major planned changes; ii) Set up the few indicators that they want to keep consistent track of and their outside data streams; iii) Receive weekly or monthly reminders to track; iv) Track their own observations; v) Pull in outside data; vi) See if anything major is changing; vii) Keep tabs on what is happening with their condition; viii) Contribute their insights to their community's research needs; ix) Share info with their doctor at scheduled visits; x) Keep track of what they've learned; and/or xi) If needed, update the plan.

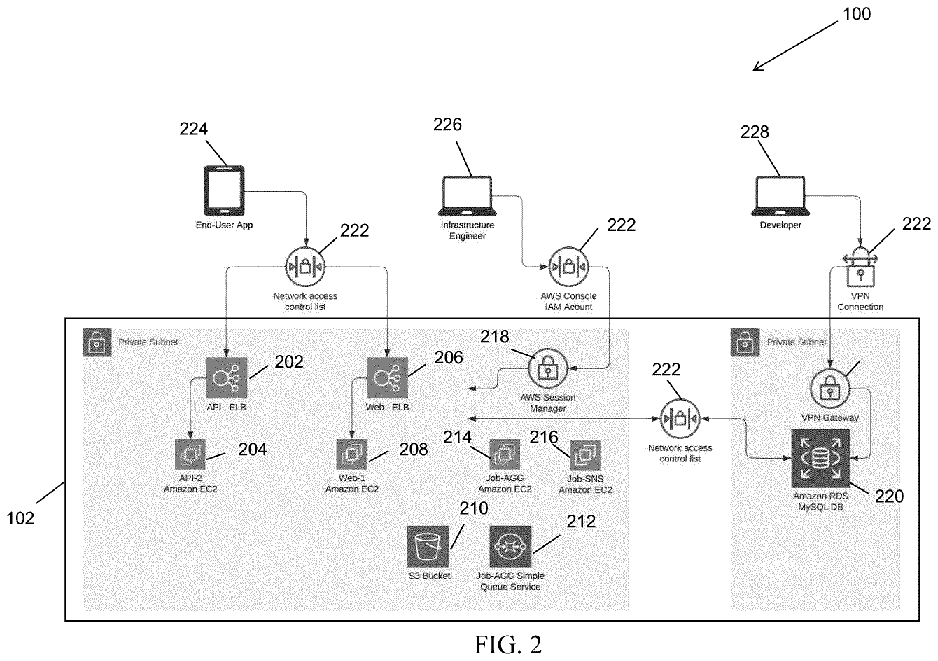

FIG. 2 illustrates another example of PAEP system 100. In the example shown in FIG. 2, device 102 is implemented by a load balancing component 202 (e.g., API-ELB) in communication with a cloud computing component 204 (API-2 Amazon EC), a load balancing component 206 (e.g., Web-ELB) in communication with a cloud computing component 208, a database 210 (e.g., a simple storage service (S3) bucket), a message queue component 212 (e.g., a simple queue service (SQS) message queue), an aggregator component 214, a notification component 216 (e.g., simple notification service (SNS) messaging service), a session manager component 218, and a database 220 (e.g., a relational database (RDS) for MySQL (structured query language)). Various network connection, gateway, identity and access management (IAM), and/or control list components 222 are also provided. As shown in FIG. 2, certain devices in communication with device 102 include a user device 224 (e.g., 108-112), an information technology (IT) infrastructure device 226, and a developer device 228.

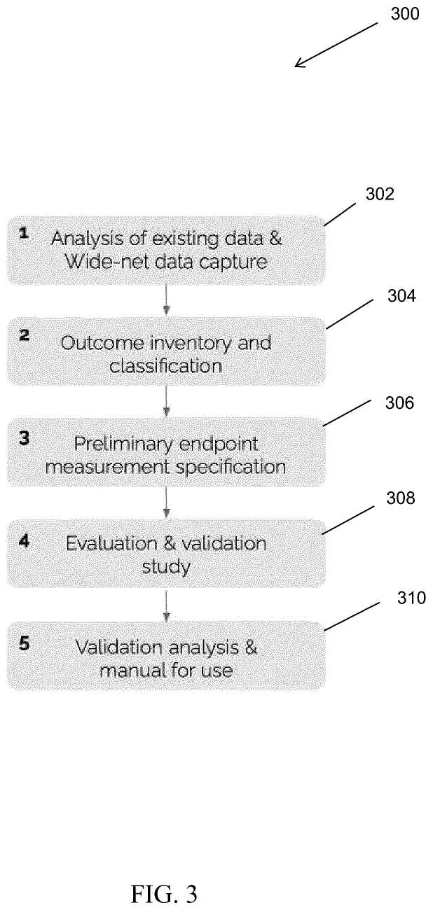

FIG. 3 illustrates an example process 300 for developing and utilizing participant-adjusted endpoints as implemented by PAEP system 100. Generally, process 300 includes, capturing 302 data with a wide-net, enumerating 304 an outcome inventory, measuring 306 preliminary endpoints, capturing 308 structured data, and analyzing 310 the captured data.

For example, to optimally collect data for the development of a participant-adjusted endpoint, natural history of a disease may be considered. When there is a strong existing understanding of the natural history, including a broad inventory of outcomes experienced by patients (e.g., including those that are less common in the population), it is possible to begin directly with structured data capture for the development of a PAEP.

For condition communities with a more nascent understanding of the natural history of the disease, wide-net data capture may be implemented to establish a broad inventory of outcomes that define the patient experience of a condition. Wide-net data capture methods may include patient interviews, literature reviews, and/or consultation with experts in the condition to establish the outcome inventory. Enabling participants to express the full scope of their individual experiences (e.g., via PAEP system 100), including outcomes that may not be obviously directly related to the condition-of-focus, is key.

From this wide-net data capture, an outcome inventory is developed (e.g., a listing of all of the outcomes experienced by this population). Based on the wide-net data capture results, two attributes of each outcome in the full outcome inventory can be estimated: (1) popularity and (2) frequency. Utilizing the estimates and a 2×2 matrix, the estimates are grouped outcomes into four tiers which can be used to establish a map of all outcomes-of-interest in the population, and to suggest the most relevant collection methods for each individual outcome. Certain outcomes are experienced as outcomes-of-interest for the entire population, while some outcomes are crucial to a smaller proportion. This concept is important for properly implementing PAEPs, because while each patient may be seeing a positive impact of a therapy, the specific indicators for “improvement” may vary person to person.

For this 2×2 system, the first axis is popularity or commonality (e.g., how many people experience it), scored from popular to less popular; the second is frequency (e.g., how often this outcome is generally experienced), scored from routine, occurring on most days, to exception, occurring less frequently. All outcomes are assigned to one tier, with the breakdown as follows: Tier A: Popular and Routine;

Tier B: Less Popular and Routine; Tier C: Less Popular and Exception; and Tier D: Popular and Exception.

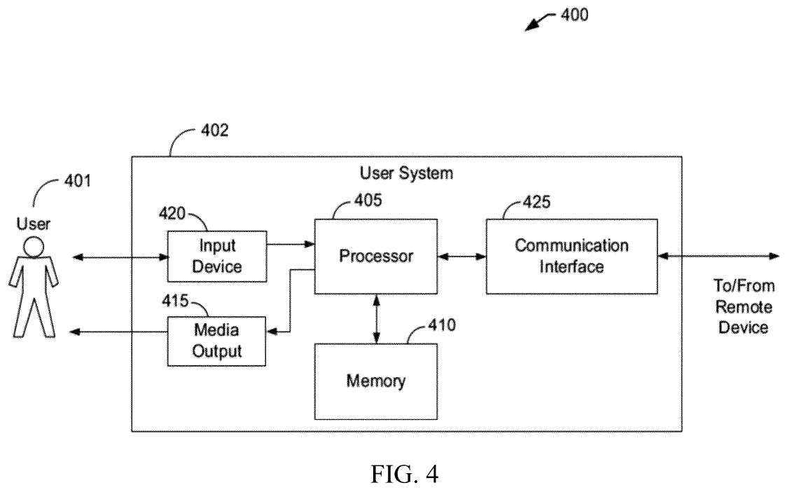

FIG. 4 illustrates an example configuration of a user system 402 operated by a user 401. In the example embodiment, user system 402 may be used to implement devices 108-112 (shown in FIG. 1), and may be used by user 401 to interact with PAEP computer device 102 (also shown in FIG. 1). In the example embodiment, user system 402 includes a processor 405 for executing instructions. In some embodiments, executable instructions are stored in a memory area 410. Processor 405 may include one or more processing units, for example, a multi-core configuration. Memory area 410 is any device allowing information such as executable instructions and/or written works to be stored and retrieved. Memory area 410 may include one or more computer readable media.

User system 402 also includes at least one media output component 415 for presenting information to user 401. Media output component 415 is any component capable of conveying information to user 401. In some embodiments, media output component 415 includes an output adapter such as a video adapter and/or an audio adapter. An output adapter is operatively coupled to processor 405 and operatively couplable to an output device such as a display device, a liquid crystal display (LCD), organic light emitting diode (OLED) display, or “electronic ink” display, or an audio output device, a speaker or headphones.

In some embodiments, user system 402 includes an input device 420 for receiving input from user 401. Input device 420 may include, for example, a keyboard, a pointing device, a mouse, a stylus, a touch sensitive panel, a touch pad, a touch screen, a gyroscope, an accelerometer, a position detector, or an audio input device. A single component such as a touch screen may function as both an output device of media output component 415 and input device 420. User system 402 may also include a communication interface 425, which is communicatively couplable to a remote device, such as PAEP computer device 102. Communication interface 425 may include, for example, a wired or wireless network adapter or a wireless data transceiver for use with a mobile phone network, Global System for Mobile communications (GSM), 3G, or other mobile data network or Worldwide Interoperability for Microwave Access (WIMAX).

Stored in memory area 410 are, for example, computer readable instructions for providing a user interface to user 401 via media output component 415 and, optionally, receiving and processing input from input device 420. A user interface may include, among other possibilities, a web browser and client application. Web browsers enable users, such as user 401, to display and interact with media and other information typically embedded on a web page or a website from PAEP computer device 102. A client application allows user 401 to interact with a server application from PAEP computer device 102.

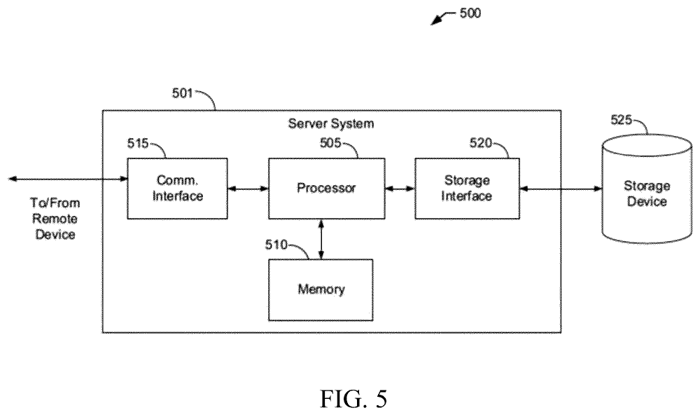

FIG. 5 illustrates an example configuration of a server system 501. Server system 501 may be used to implement PAEP computer device 102 (shown in FIG. 1), for example. Server system 501 includes a processor 505 for executing instructions. Instructions may be stored in a memory area 510, for example. Processor 505 may include one or more processing units (e.g., in a multi-core configuration) for executing instructions. The instructions may be executed within a variety of different operating systems on server system 501, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or other suitable programming languages, etc.).

Processor 505 is operatively coupled to a communication interface 515 such that server system 501 is capable of communicating with a remote device such as user system 402 (shown in FIG. 4) or another server system 501. For example, communication interface 515 may receive requests from, and send results to, devices 108-112 via the Internet, as illustrated in FIG. 1.

Processor 505 may also be operatively coupled to a storage device 525. For example, storage device 525 may be used to implement databases 210, 220 (shown in FIG. 1) and/or other databases described herein. Storage device 525 is any computer-operated hardware suitable for storing and/or retrieving data. In some embodiments, storage device 525 is integrated in server system 501. For example, server system 501 may include one or more hard disk drives as storage device 525. In other embodiments, storage device 525 is external to server system 501 and may be accessed by a plurality of server systems 501. For example, storage device 525 may include multiple storage units such as hard disks or solid state disks in a redundant array of inexpensive disks (RAID) configuration. Storage device 525 may include a storage area network (SAN) and/or a network attached storage (NAS) system.

In some embodiments, processor 505 is operatively coupled to storage device 525 via a storage interface 520. Storage interface 520 is any component capable of providing processor 505 with access to storage device 525. Storage interface 520 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 505 with access to storage device 525.

Memory area 510 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program. Cloud embodiments are also envisioned.

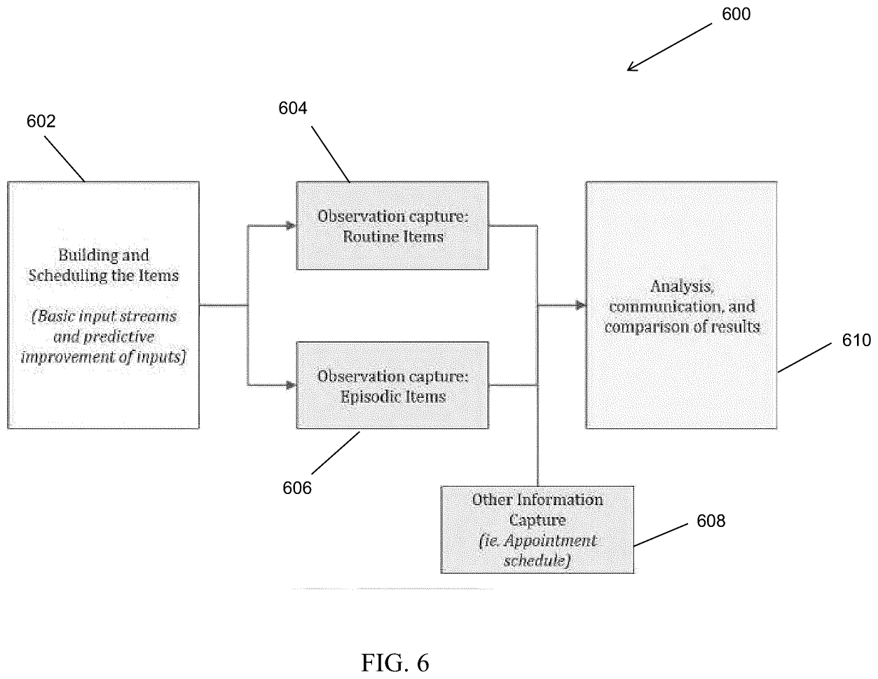

FIG. 6 illustrates an example information capture process 600 implemented by PAEP system 100.

In the example embodiment, system 100 is designed to help caregivers and patients capture, communicate, and compare health and behavior observations. To capture an observation, device 102 builds and schedules items 602 (e.g., basic input streams and predictive improvement of inputs). A user (e.g., at one of devices 108-112) then responds to item 602 (e.g., a bundle of multiple-choice questions designed to help the user record the details of that observation and generated by device 102). There are at least 5 types of observations supported by the device 102, and each has a corresponding Item type. The 5 item types are: (1) Progress; (2) Symptoms; (3) Medications; (4) Treatment Behaviors or Habits; and (5) Measurements. These items can be accessed by the user through one of two observation capture flows: routine flow 604 or episodic flow 606. Routine flow 604 is schedule-based, and designed to allow users to regularly keep track of a particular Item. Episodic flow 606 allows for one-off item responses by the user, and does not have a scheduling component.

As an example, a parent at one of device 108-112 may set up a symptom (e.g., by communicating with device 102): headache item as a routine, as they want to remember to track whether or not their child has had a headache on any given day. They will select an appropriate frequency and timing for that item (e.g., daily, at dinnertime). On a dashboard of a graphical user interface (GUI) generated by device 102, that parent will receive a prompt on a daily basis around 6 pm (dependent on her dinnertime setting) to respond to the symptom: headache item. If their daughter has had a headache, the parent will be asked a series of multiple-choice questions designed to gather details on the headache—these details are called characteristics, and might include how bad the headache was, how long it lasted, and whether it was accompanied by nausea.

However, if their daughter does not frequently get headaches, the parent might not have a symptom: headache item set up as a routine. In this case, the parent could still go through the same observation capture sequence—they would simply access the symptom: headache item through episodic flow 606, rather than by responding to a scheduled prompt. In episodic flow 606, symptoms, medications, and treatment behaviors or habits are generally grouped together into one expanded item, as the user is often looking to track a symptom together with the medications and/or treatment behaviors that were used to react to that symptom. Because of this, in episodic flow 606, the example parent would receive the headache characteristic questions, followed by a treatment question—which medications and/or treatment behaviors have they used to treat the symptom, if any? System 100 allows for pre-set episodic symptom tracking pathways, which allow the user to quickly move through tracking a symptom that they experience frequently, complete with user-specific characteristic questions and pre-set treatments for the symptom.

Outside of items, users can also capture non-observation information 608 about their care. This information is captured in order to help the user better organize their health information, and in some cases, to help communicate the observation results. Examples of this type of non-observation information include details on the healthcare providers and external caregivers (like schools) that interface with the patient, and a log of scheduled and completed healthcare appointments.

Upon gathering the data described herein, device 102 then processes 610 the data via analysis, communication, and comparison of results, as explained herein in further detail.

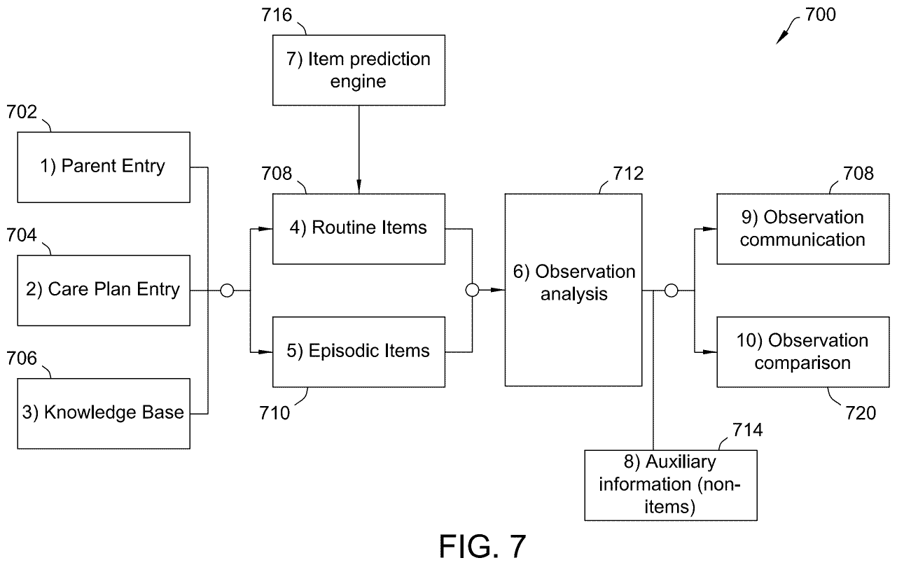

FIG. 7 illustrates an example process 700 for building and scheduling items (e.g., building and scheduling item 602) as implemented by PAEP system 100. As shown in FIG. 7, process 700 includes parent entry 702, care plan entry 704, knowledge base 706 (e.g., at database 210 or 220), routine items 708 (e.g., associated with routine flow 604), episodic items 710 (e.g., associated with episodic flow 606), observation analysis 712 (e.g., associated with processing 610), auxiliary information 714 (e.g., associated with information 608), an item prediction engine 716, observations communication 718 (e.g., associated with processing 610), and observations comparison 720 (e.g., associated with processing 610).

In the example embodiment, there are three main input streams that feed the creation of routine and episodic items. These input streams are parent entry 702, care plan entry 704, and knowledge base 706. Through parent entry 702, as the name suggests, parents (or other individual users) can complete a guided questionnaire to create a routine item.

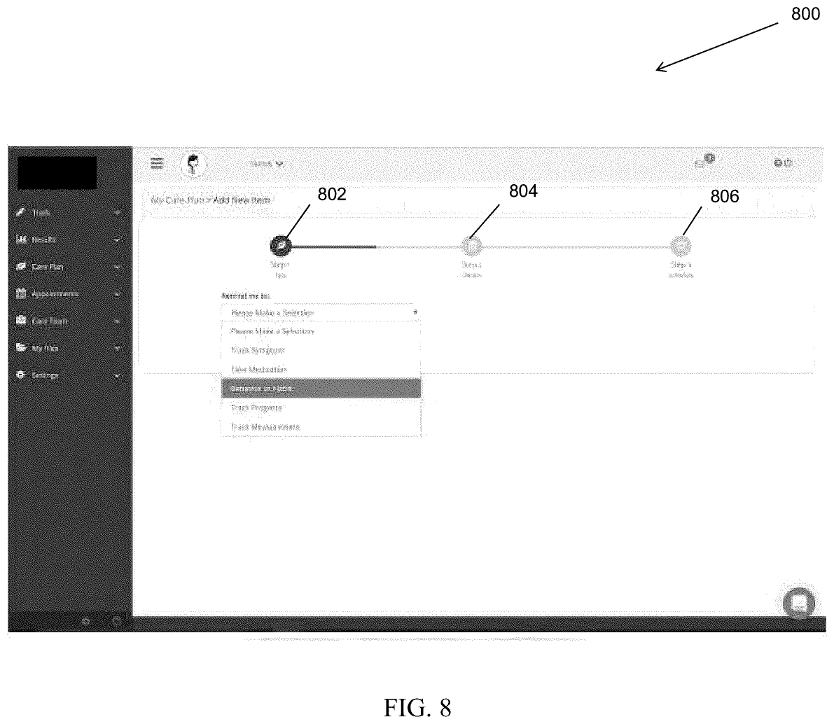

FIG. 8 illustrates an example GUI 800 generated by PAEP system 100 with an example questionnaire. As shown in the FIG. 8, parent entry 702 is a 3-step process. In step one 802: the user selects an item type (e.g., track symptom, track medication, behavior or habit, track progress, track measurement, etc.).

In step two 804, the user enters the details of that item. For example, if the user is adding a new behavior or habit, they would search for the correct behavior from the behaviors available in database 210 or 220, and then would enter the dose amount and frequency. In the case of a behavior, this is equivalent to amount of time or number of sessions required to complete the behavior, and how often the behavior should occur (e.g., in the event that the user searches for a behavior and can't find the correct one, they can add their own). This new behavior will be added to their local list of behaviors, and will also be added to a staging area that allows for determination as to whether the new behavior should be added to the overall list stored at database 210 or 220).

In step three 806, the user sets the schedule for the new item. There are several scheduling options, such as: daily time-of-day scheduling, daily specified-time scheduling, and interval scheduling. Daily time-of-day scheduling allows the user to specify certain periods during the day (e.g., breakfast, lunch, bedtime, etc.) at which they would like to receive a reminder for this item. Daily specified-time scheduling is more precise, allowing the user to choose an exact time to receive a reminder. Finally, interval scheduling makes it possible for the user to set regular intervals at which they will receive a reminder (e.g., once every 12 hours, or once every 2 weeks). The interval begins at a date and time specified by the user. Once step three is completed, the new routine item will be displayed on GUI 800 on the specified days, and the user will receive alerts by text, email, or mobile application alert at the chosen times.

Returning to FIG. 7, the second stream of item creation is care plan entry 704, a mediated process that allows users to upload information on a change to the care plan (e.g., which describes the treatments and tracking required to manage the patient's health) and see these changes reflected in the routine and episodic items available to them in system 100. A user can initiate care plan entry using either a web or mobile application provided by device 102 (e.g., at devices 108-112), and is prompted to do so at the end of every appointment that is scheduled in their profile at system 100.

To create a care plan request, the user simply notes the reason for the changes (e.g., doctor's appointment, phone call with a provider, their own change) and then uploads all supporting documentation to system 100 for storage. This documentation can include audio or video of the healthcare provider or the user explaining changes to the care plan; photos or scans of documents from the healthcare provider; or photos, scans, or Word documents containing the user's notes. Upon uploading the supporting documentation, the user is prompted to add any commentary to explain the care plan changes, and the user-facing side of care plan entry is completed with the submission of the care plan request.

Once a user submits the care plan request, an administrator of system 100 may initiate care plan entry, through which the new routine items and pre-set episodic symptom tracking pathways for the user can be built. Care plan entry is a stepwise, online process that is part of an administrator web interface in system 100. In the example embodiment, the process has 5 steps: i) renew routines and episodes (e.g., renew or retire all existing routine items and all pre-set episodic symptom tracking pathways) —for example, a user may renew yoga (every day, lunchtime), renew Asmanex (2× per day, breakfast and bedtime), renew headache tracking pathway, renew coughing tracking pathway, and retire Xopenex (as needed); ii) update renewed routines (e.g., for those routine items that were renewed, review the details and update as necessary—for example, a user may change yoga (every day, lunchtime) to yoga (every day, 07:30 am); iii) Add new routines (e.g., create new routine items, as specified by the care plan request—for example, add: exercise, 3× per week, 20 minutes); iv) update renewed episodes (e.g., for those pre-set episodic symptom tracking pathways that were renewed, review the details and update as necessary—for example, change coughing tracking pathway—if severity >4, take Albuterol (2 puffs)); and v) add new episodes (e.g., create new episodic symptom tracking pathways, as specified by the care plan request—for example, a user may add: wheezing tracking pathway, with the following characteristics and treatments . . . (etc.)).

The third stream of item creation is via knowledge base 706. Knowledge base 706 is database (e.g., included in database 210 or 220) of possible items that may be tracked by patients, with the ability for individual items to be assigned to condition- and age-based profiles, for easy suggestion to users who fit those profile types. For example, the knowledge base 706 includes an item called “headache,” which has multiple associated characteristics, including severity and accompanying nausea. Headache can be assigned to multiple different condition- and age-based profiles. For example, it might be assigned to the condition profile for migraine, in all ages. When a new user signs up in system 100 and chooses migraine as a condition, they will be prompted to add headache as a routine symptom to track.

Knowledge base 706 includes tables for all major item types, and is constantly updated by device 102 in reaction to changing guidelines and to user and physician requests. When a user adds a new item that is not included in knowledge base 706, the new item is reviewed and, if appropriate, will be added to knowledge base 706. In this way, the knowledge base 706 remains optimally up-to-date, and even ahead of current institutional medical knowledge.

Once either routine or episodic observations have been captured, they are automatically subject to a series of analytics designed to produce easily comprehensible feedback for the user. This level 1 analysis, which includes readable metrics (e.g., see below) and basic bar and pie charts, are displayed on the main page of the web app, and may also be accessed through the navigation on both the web application and the mobile application by clicking on “Results”. Level 1 analysis is designed to keep the user up-to-date with the current status of the patient's tracked health outcomes, care plan adherence, and overall wellness, as a summary of all observations captured by the platform. The time-series of Level 1 Analysis may be bounded by the last two weeks.

Readable metrics are phrase- and sentence-format results designed to be easily understood by the user who is unfamiliar or uncomfortable with graphical representations of data. For example, if a user has tracked use of their child's Asmanex inhaler for 73% of scheduled Asmanex items, they would see the following readable metric on the main page: “We usually remember to take Asmanex (73%)”. As another example, if this same user had reported 55% of days to be good days through the general progress item, they would see, “Overall, Jessica is feeling pretty good. 55% of days were good days, and the top reason for good days is that Jessica is enjoying school. The top reason for bad days is that she is not sleeping well.” These readable metrics are built by matching the calculated score for an individual metric, like Asmanex adherence, to a prefabricated phrase, like “We usually remember to take . . . ”. These phrases are updated based on consumer feedback, and in some embodiments, an artificial intelligence component of device 102 (e.g., similar to a chatbot), allowing for faster iteration based on this feedback.

For communication with outside stakeholders, including doctors and other care team members, device 102 utilizes level 2 analysis, which incorporates a longer timeframe and correlation calculation across observation types. For instance, level 2 analysis might include an analysis of the correlation between missed medication and a symptom, or the converse, which might suggest a side effect. The results of level 2 analysis are then presented in the appointment guide (e.g., a PDF document generated by device 102 that includes level 1 and level 2 analysis results), along with non-observation information, that is organized to optimally facilitate a problem-solving conversation between the caregiver/patient and the healthcare professional.

Finally, level 3 analysis is designed to establish links across patients and patient populations. This comparison analysis will be a great value-add to the medical community, as many of the datatypes that will be captured through the use of device 102 are not currently available in large quantities across patient populations, and will be useful in determining the efficacy of treatment plans for many chronic conditions. Over time, this level 3 analysis will be used to help direct research and determine the best course of treatment for an individual patient, based not only on their condition but on other factors captured by the platform, including the symptoms that they are experiencing and the success of previous treatments.

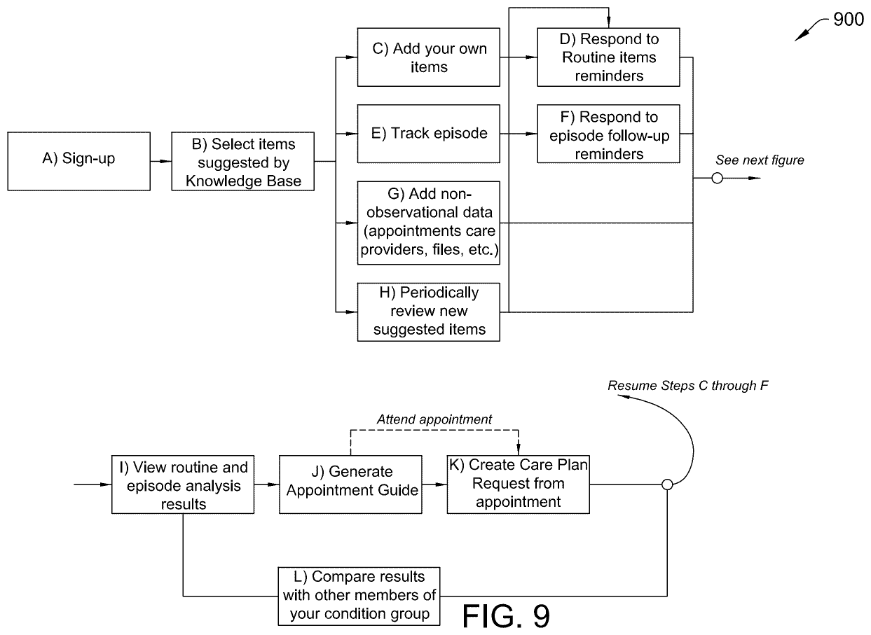

FIG. 9 illustrates another example method 900 for developing and utilizing participant-adjusted endpoints as implemented by PAEP system 100 as described above in further detail.

In a summary of a portion of the example embodiment, PAEP system 100 includes a cloud-based web and mobile application with advanced analytic capabilities designed to optimize the ease and accuracy with which patients and caregivers can capture health observations. It includes three main components: i) build and scheduling of items (e.g., 602); ii) item-based observation capture and other non-observation information capture (e.g., 604-608); and 3) observation analysis, communication, and comparison (e.g., 610). System 100 provides a new set of data on health outcomes and treatment plan adherence that will help to improve the state of chronic disease care, for both users of system 100 and for patients impacted by the results of studies using system 100.

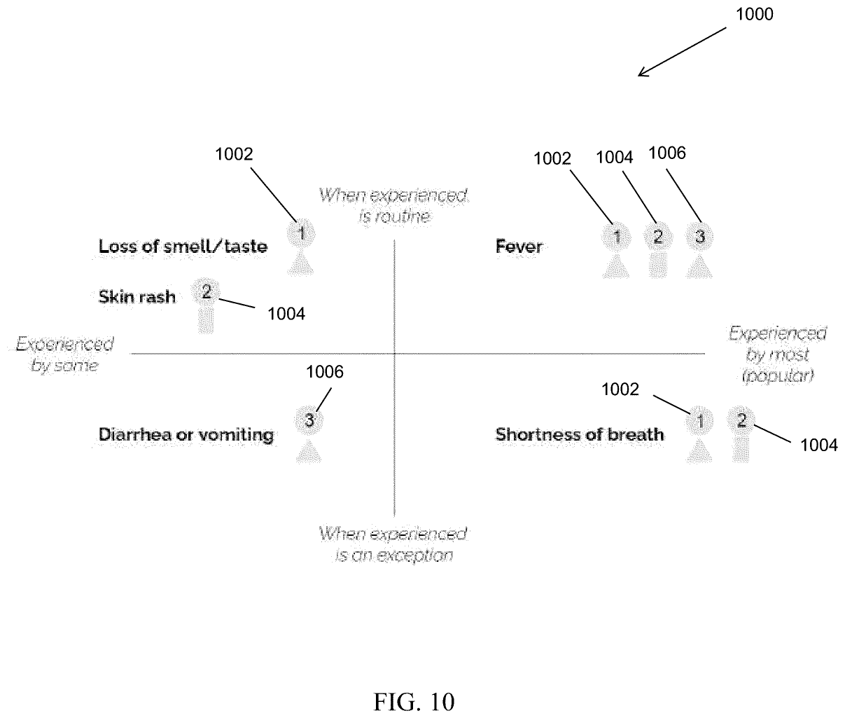

FIG. 10 illustrates an example commonality and frequency graph 1000 illustrating information utilized by PAEP system 100 (e.g., with respect to COVID-19). In the COVID-19 example, the way in which coronavirus infection

affects each person differently makes this acute condition a compelling case for the

use of PAEP system 100 to understand the efficacy of potential therapies. Take three example individuals, each exhibiting some overlapping and some unique symptoms to the rest of the group. Patient one 1002 has fever, loss of smell/taste, and shortness of breath, patient two 1004 has fever, skin rash and shortness of breath, and patient three 1006 has fever and diarrhea or vomiting.

In this example, all three patients with COVID symptoms experience a fever. In addition to the common outcome-of-interest, they each exhibit more individual expressions of the disease, like diarrhea, vomiting, or shortness of breath. This is an example of variation in the disease manifestation. To fully understand the efficacy of a new therapy for COVID-19 in this example population, a way to measure not only change in fever (the common outcome), but also change in diarrhea for Patient 3, improvement in smell/taste for Patient 1, etc. is needed. Accordingly, utilization of PAEP system 100 is favorable.

In the example above, fever is experienced regularly (routinely) by the whole population, making this a “Tier A” symptom. Loss of smell/taste, which occurs regularly but only by a small number of people, lands in “Tier B”. Diarrhea or vomiting is only experienced by a small number and is experienced as an exception rather than routine, and so is “Tier C”. Finally, shortness of breath is experienced by a majority of the population, but is not routine, and so is a “Tier D” outcome.

Once an outcome inventory has been established and a first estimate of tiering has been completed (e.g., above), structured data capture can be achieved using a combination of the following methods: i) home-reported outcome (HRO) data capture (e.g., see below); ii) electronic patient-reported outcome (ePRO) capture; iii) clinical endpoint capture; iv) text-based data collected by participants using a guided notebook or by responding to a questionnaire; and/or v) interview-based data capture via structured interviews.

Certain clinical endpoints and patient reported outcome (PRO) instruments may be used as components of the PAEP data collection method implemented by PAEP system 100. However, in some cases, supplementing with HRO or other data capture methods that allow for variation in the set of data-points captured by each individual participant are optimal.

For example, in the example embodiment, HROs are patient and caregiver-generated data on symptoms and behaviors that are captured outside the context of the clinic, utilizing multiple-choice and multi-select questions generated by device 102, in a manner that is designed for interchangeable capture of individual outcomes (no interdependence, unlike most PRO questionnaires). They allow for the detection of change in both common outcomes in a population and more personal outcomes experienced by participants. The content of individual HRO question and answer text is held constant across a population, but the assortment of HRO questions completed by an individual is specific to that participant.

HROs capture the observations made by patients and family caregivers in the course of their everyday lives. HROs are ideally collected real-time or with a short recall period of less than 10 days. The intention is for these data to reflect the changes in health observed by a patient or caregiver outside of clinical encounters.

Unlike most PROs, HROs are not primarily collected as one-piece surveys, where questions are dependent on each other and must be administered together in the same order for each participant. HROs are designed as individual data-points, and the data-points themselves (like coughing severity 1-10 or presence of headache) act independently of one another. A set of HRO questions can be the same across a population (a group of 50 patients all tracking the same 5 HRO items), but also have built in flexibility. For example, in the group of 50 patients, all might track those 5 HRO items, but a subset of the group may decide to add something else that is of

particular use to them.

The HRO allows for much greater flexibility in data collection, enabling patients and caregivers to capture insights most relevant to them. This, in turn, improves the experience and associated tolerability of long-term self-assessment, leading to longitudinal dataset development.

Given the less-standardized nature of HRO collection, in most cases a patient-specific baseline for each HRO item being studied will be established by device 102. Population baselines may also be useful, but a patient-specific dataset lends itself well to the personal threshold analytical methods used increasingly to augment precision, such as in cancer care and research.

The form of the HRO question is primarily determined based on the relative frequency of the outcome being studied. If an outcome occurs with frequency in participants who experience it, and so is ‘routine’, the associated HRO question enables capture of a gradient of experience (e.g., including severity (1-10), count-per-day (integer), or multiple-choice (option-based)). If the outcome is less frequent, and so is ‘episodic’, it is a candidate for a simple Yes/No HRO question framing, wherein analysis focuses on absolute frequency rather than a gradient of experience with each episode.

For instance, an example HRO data specification is provided below in Tables One and Two (e.g., to illustrate the components of HRO data capture for a particular condition or area of study). Based on scores in the outcome inventory, domain vs. domain weighting, and tiering system results, a data spec with the following sections is implemented:

TABLE ONE
Home-Reported Outcomes Requested from all Participants

| | | | |
|---|---|---|---|
| Domain, | Question | Question Type | Tracking Schedule |
| Outcome | Text | (Y/N, 1-10, | (routine (with |
| | | multiple choice, | schedule), quick- |
| | | multi-select, count) | track) |

TABLE TWO
Home-Reported Outcomes Suggested, Selected by Individual
Participants

| | | | |
|---|---|---|---|
| Domain, | Question | Question Type | Tracking Schedule |
| Outcome | Text | (Y/N, 1-10, | (routine (with |
| | | multiple choice, | schedule), quick- |
| | | multi-select, count) | track) |

Once the outcome inventory is established, domains are assigned to group the outcomes into topic areas. Domains may also be assigned a priori, drawing from previous expertise in the condition. This grouping facilitates understanding of general trends within the outcomes that are reported by participants, and easy exaltation the impact of therapy across different topic areas. In the example embodiment, analysis of results for a participant-adjusted endpoint should include both outcome-level and domain-level analysis.

For instance, in a cystic fibrosis example, both respiratory outcomes (e.g., coughing, wheezing, shortness of breath, etc.) and gastrointestinal outcomes (e.g., bowel movement count and characteristics, nausea, etc.) are both important. “Respiratory” and “Gastrointestinal” are outcome domains in this example.

In most cases, each outcome should only be assigned to one domain. In circumstances in which the domain for an outcome is unclear, that outcome should be annotated with the additional potential applicable domain(s). Domain-level analysis can then be conducted with different versions of the domain assignment, to assess whether the assignment of this outcome has an effect on the overall domain-level results.

TABLE THREE
Domain vs. Domain Weighting

| | Relative Domain Weight |
|---|---|
| Domain 1 | 60 |
| Domain 2 | 30 |
| Domain 3 | 10 |

Trial designs can be experimental, where researchers randomly assign an intervention or therapy to approximately one half of participants (e.g., a treatment group) and use a placebo or the current gold standard treatment (e.g., control group) for the other half of participants.

A HRO data-spec benefits from this type of study design as outcomes are more easily analyzed, but can be great in any setting where some type of comparator exists. Two examples of possible comparators would be pre-intervention vs. post-intervention outcomes or observed vs. expected outcomes.

Regardless of experimental design, HROs compliment longitudinal studies because the data is collected on a daily or weekly basis. This creates a higher amount of granularity in the data as clinic-based outcome collection requires participants to travel or visit a healthcare professional. This coordination with healthcare facilities causes traditional data collection methods to be more infrequent and only collected data on a monthly to several times a year basis.

A first step in analysis is an exploratory data analysis (EDA) phase. The goal of this step is to summarize the main characteristics of the data, often visually, and seeing what the data says (e.g., outside of rigorous hypothesis testing). This step provides insights into patient responses outside of what is expected. Example EDA steps implemented by PAEP system 100 are as follows with example outputs.

A first step includes creating a table of all domains with number of outcomes, people, tracks, and positive or negative changes, as shown in Table Four.

TABLE FOUR

| | | | | | |
|---|---|---|---|---|---|
| D1 | O₁ | X₁/Y (%1) | T₁ | p₁/O₁ | n₁/O₁ |
| D2 | O₂ | X₂/Y (%2) | T₂ | p₂/O₂ | n₂/O₂ |
| D3 | O₃ | X₃/Y (%3) | T₃ | p₃/O₃ | n₃/O₃ |

A second step includes tabling of all outcomes with number of people, number of tracks, and number of positive or negative change, as shown in Table Five.

TABLE FIVE

| | | | Participants | Participants |
|---|---|---|---|---|
| | | | with at least 1 | with at least 1 |
| | Number of | Total | positive | negative |
| Outcomes | people | tracks | change | change |
| Outcome 1 | N₁ | T₁ | P₁/N₁ | n₁/N₁ |
| Outcome 2 | N₂ | T₂ | P₂/N₂ | n₂/N₂ |
| Outcome 3 | N₃ | T₃ | P₃/N₃ | n₃/N₃ |

A third step includes generating a histogram of changes across each domain. A fourth step includes generating a histogram of each participant with number of outcomes, number of positive changes and number of negative changes.

Statistical analysis by device 102 typically includes one or more of the following frameworks: i) participant; ii) domain; and iii) outcome results. For example, a comparison group (e.g., one or more of a pre-post or control vs. treatment group is defined. The main objective then is to use the results from all participants, domains, and outcomes to assess in what areas people showed improvement.

What constitutes “improvement” or “significant change” can be highly variable depending on the condition space, previous research, and study design. All of the above conditions may be analyzed, as analysis may follow the same fundamental design. Statistical tests for participant adjusted endpoints will use a comparison method such as two-sample t-test, regression, and chi-square to compare observed and expected results.

In participant analysis, the broad outcomes across the whole population and/or one individual's progress over the course of the study are compared. This may resemble traditional clinical trial analysis. Comparing several different outcomes simultaneously either in a population or individual over time allows device 102 to control for different characteristics in the population.

In domain analysis, a goal is to compare each domain specifically across the population, and see how many people improved. By examining each domain individually, device 102 compares those with similar outcomes, but could with small differences in either type, outcome, or frequency. This is because a domain level analysis takes a group of symptoms into account rather than just one specific item.

Further, for outcome analysis, a goal is to compare all outcomes in a comprehensive outcome list. In this analysis, device 102 determines what outcomes saw the most improvement across everyone who experienced those outcomes. The benefit of this analysis is that each outcome can manifest differently, and device 102 achieves the granular detail from the HROs.

An example consideration deals with missing data, or instances where device 102 has information about certain participant outcomes. As mentioned earlier, HRO datasets typically collect data at more regular and frequent intervals than traditional clinic-based data collection. This means missing data could be less detrimental to overall study success than normally.

A next consideration is people responding at different intervals and times (e.g., how does device 102 compare one person who records their experiences every day, to someone who records twice a week). One way device 102 handles this situation is to standardize everyone's responses to the same amount (e.g., perhaps over that week device 102 averages each person's outcomes so they are on the same time scale).

Further, device 102 controls for baseline severity. Individuals with greater overall disease severity have more opportunities to improve their outcomes compared to those with better overall health.

Additionally, the framework for power calculations can be tricky. Sample size is highly dependent on various assumptions device 102 may make and how much change is necessary for “significant change”.

Another consideration for device 102 is that people record different outcomes from one another, a common occurrence when using participant adjusted endpoints. Domain assignment, as implemented by device 102, helps with this complication, as device 102 analyzes outcomes as a slightly higher, but related level, making different outcomes more similar to each other.

Device 102 establishes a multidimensional understanding of efficacy, including variation among individuals in experience of a condition and response to therapy, in order to fully measure the benefit-harm balance. The concept of utilizing personal thresholds in concert with population thresholds for change is key to the participant-adjusted endpoint framework of system 100.

The introduction of a participant-adjusted endpoint framework by system 100 will have an outsized effect on the development of research studies that reflect the priorities of patient populations, allow for variation in the individual experience of disease, promote diversity in research, and ensure long-term applicability of resulting endpoints to a changing population, evolving therapeutic landscape, and shifting baseline state of health.

PAEP system 100 may implement an enhanced understanding of patient priorities, and is an elementally patient-driven approach to establishing endpoints for a condition. System 100 reflects a view of outcomes that has greater fidelity to the impact felt by individual patients and caregivers, and is less abstracted from the reality of living with a condition than more traditional, clinical measurements. System 100 enables patients and family caregivers to be directly involved in determining how the efficacy of a therapy for their condition will be measured, and ensure that endpoints used to make regulatory decisions for new therapies are aligned with the ultimate goals of the individuals who will receive them.

PAEP system 100 also unlocks new avenues in precision medicine, as it establishes a path for greater accuracy in the description of individual phenotypes. This, in turn, creates greater potential for discovering new clusters of phenotypes, to further subdivide existing diagnoses and to become more precise in the description of conditions to form a more precise diagnosis and follow a more precise treatment plan for the individual.

PAEP system 100 also promotes diversity in clinical research. Enablement of outcomes data capture and endpoint development that may be adjusted based on the phenotype and experiences of the individual allows for greater representation of a diverse set of participants, and decreases the need to recruit highly homogenous groups of participants in order to conduct rigorous studies of a new intervention.

Additionally, the flexibility of system 100 ensures long-term applicability to a changing population, evolving therapeutic landscape, and shifting baseline state of health. The adjustable and relative nature of system 100 assures this longevity, as the outcomes-of-interest that include an endpoint for a given condition are able to shift over time, based on the phenotypes of the individuals in the population and how their outcomes are measured.

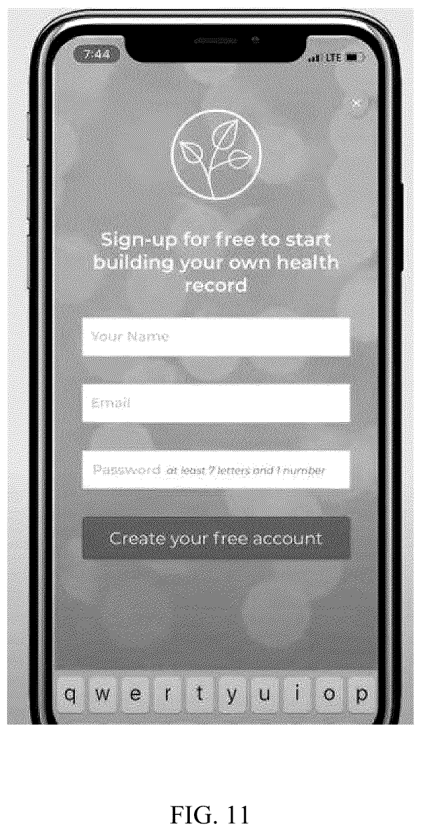

FIGS. 11-53 illustrate example screenshots and/or GUIs (e.g., as shown at one of devices 108-112 based on data received from device 102 via API 104 and/or 106) for developing and utilizing participant-adjusted endpoints as implemented by PAEP system 100. Example features of an application provided by system 100 for developing and utilizing participant-adjusted endpoints may include behavior tracking, trigger tracking, advanced scheduling, tiered tracking based on previous response, activity-based, suggested questions, smart in-application reporting, clear, readable metrics, a single place to get a summary and relevant, and timely push notifications based on user behavior. For example, the different customizations/selections made by a user as shown in FIGS. 11-53 may be analyzed by device 102 to update data in database 210 or 220 associated with the user and data in database 210 or 220 associated with the general population.

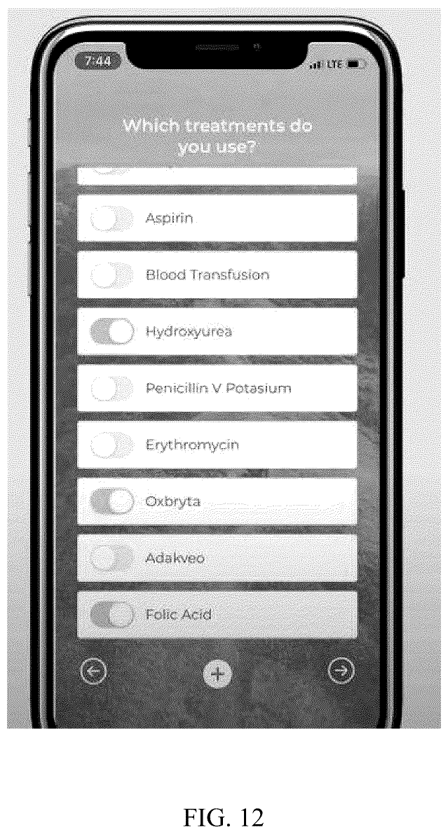

In the example shown in FIGS. 11-53, FIG. 11 illustrates an example login page at one of devices 108-112 where a user can input a name, email address, and/or password to register/create an account in system 100. FIG. 12 illustrates a next example interface asking the user which treatments they currently use. Selectors are provided adjacent each treatment, along with an add selector where a user can create their own treatment (e.g., if not already included in the list of treatments provided). As shown in FIG. 12, a user has selected Hydroxyurea, Oxbryta, and Folic Acid.

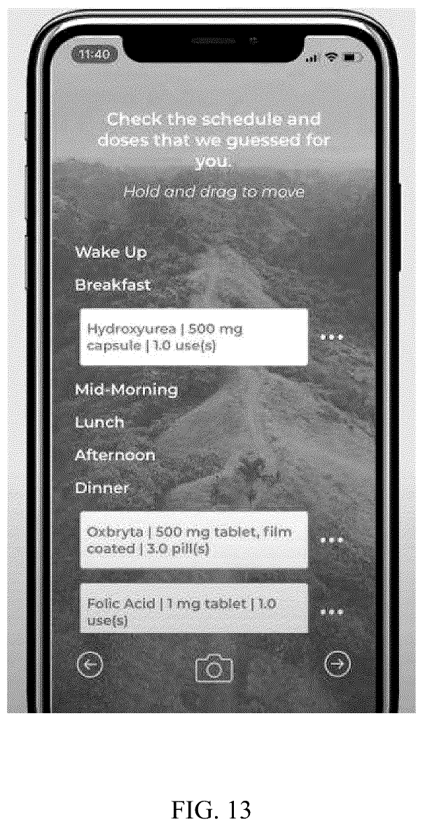

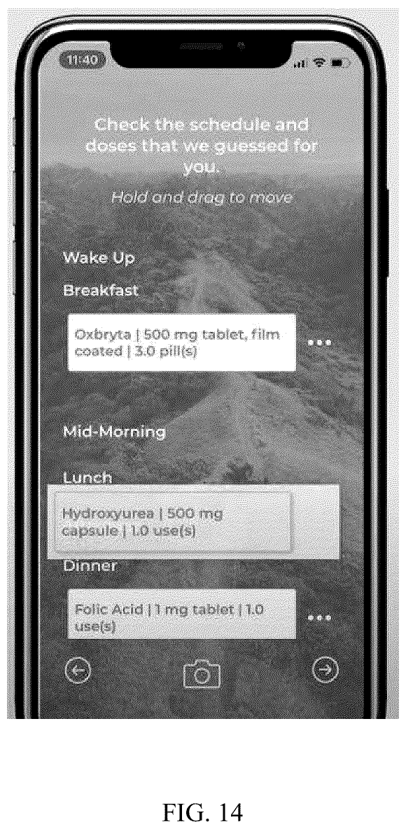

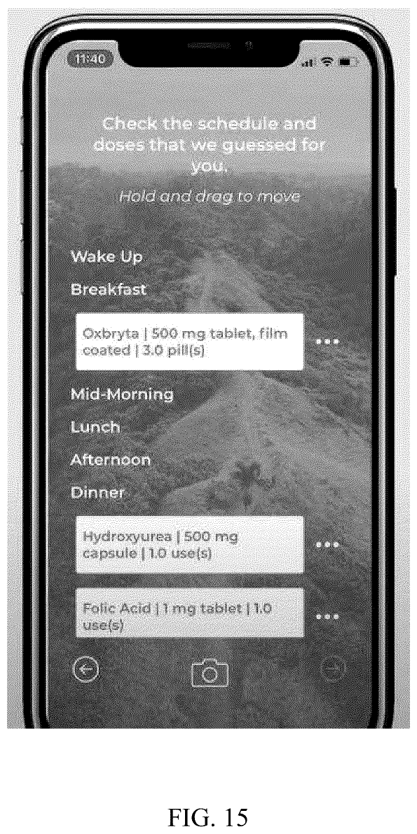

FIG. 13 illustrates a schedule automatically generated (e.g., based on data stored in database 210 or 220) by device 102 based on the inputs shown in FIG. 12. For example, device 102 scheduled Hydroxyurea at breakfast, and Oxbryta and Folic Acid at dinner. However, if desired, a user can manually change the scheduled (e.g., by dragging and dropping the treatments at the GUI). FIG. 14 illustrates the example GUI while Hydroxyurea is being dragged across the interface, and FIG. 15 illustrates the example GUI after Hydroxyurea is scheduled for dinner (e.g., instead of breakfast). As shown in FIG. 15, Oxbryta has also been moved to breakfast from dinner.

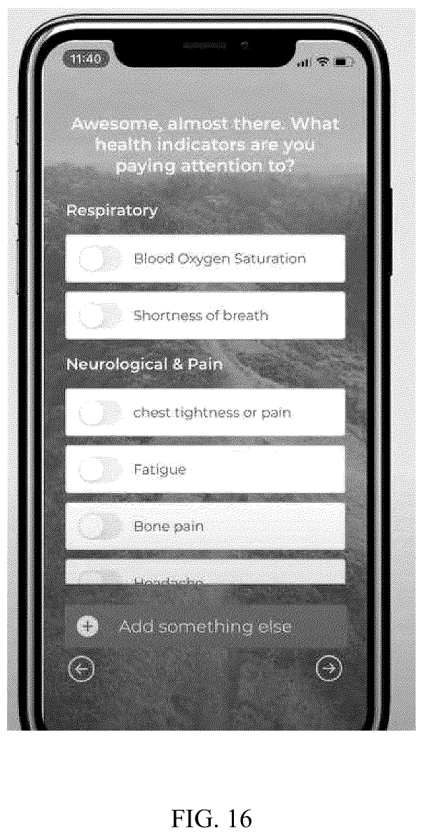

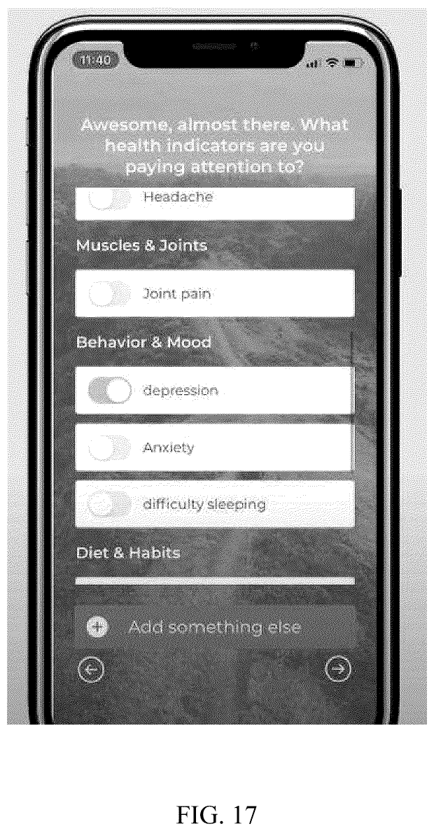

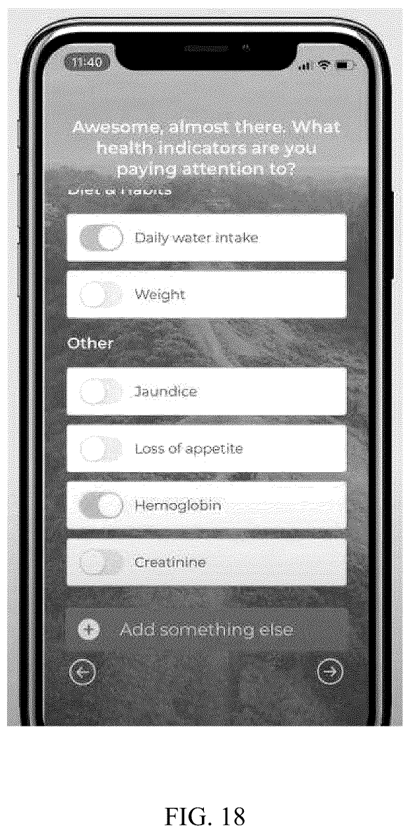

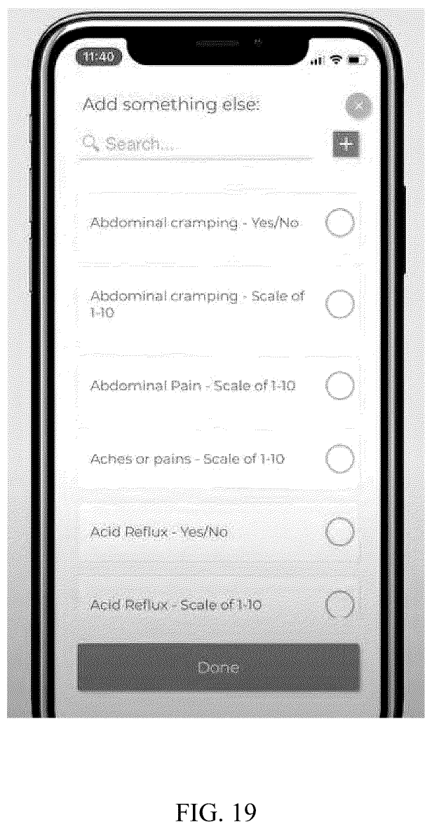

FIG. 16 illustrates a next GUI asking which health indicators the user is paying attention to. Selectors are provided adjacent each indicator for user selection, as well as an add selector where a user can create their own indicator. For instance, FIG. 17 illustrates user selection of depression, FIG. 18 illustrates user selection of daily water intake and hemoglobin, and FIG. 19 illustrates a GUI provided in response to user selection of the add selector.

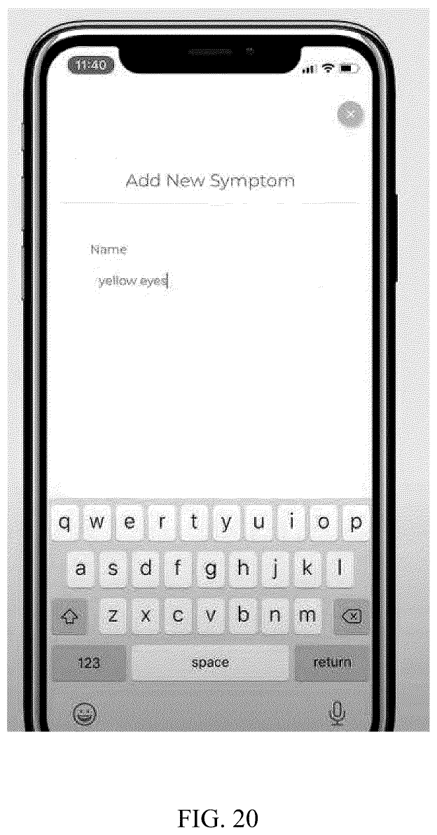

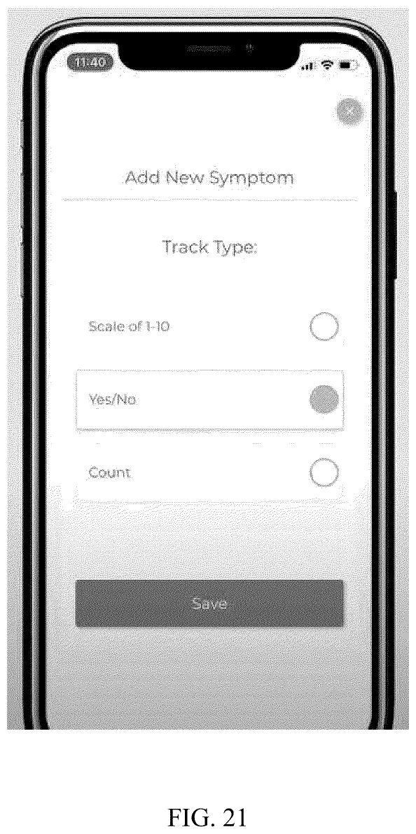

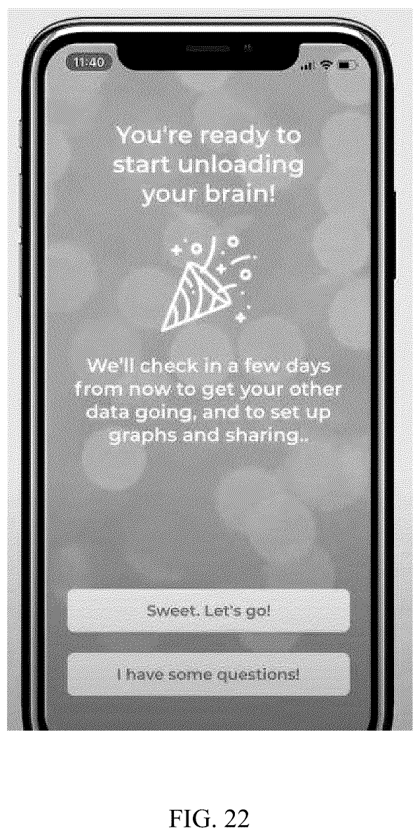

For example, in FIG. 19 certain additional indicators are provided for selection by a user, as well as an add selector for a user to create a new indicator. FIG. 20 illustrates a GUI provided in response to user selection of the add indicator along with entry of “yellow eyes” into a name field. Upon entry of yellow eyes, a type selection GUI is shown in FIG. 21. Accordingly, a user may select scale of 1-10, yes/no, count, and/or other track types. As shown in FIG. 21, the user has selected yes/no as the track type for yellow eyes. FIG. 22 illustrates an example GUI provided in response to selection creation of the yellow eyes indicator (e.g., and selection of the save selector shown in FIG. 21) and indicating that the initial setup process with system 100 is complete.

FIG. 23 illustrates an example dashboard (e.g., home page) GUI generated by device 102 that launches when a user opens an application and/or web page associated with system 100. As shown in FIG. 23, the date is provided, a “Begin Today's Questions” selector (e.g., prompting a user to being answering questions associated with the user), as well as other selectors for accessing various functionalities of the application and/or web page.

FIG. 24 illustrates a GUI provided in response to selection of the questions selector (e.g., for a user to being their daily questions). As shown in FIG. 24, the user is prompted to enter details regarding their last night's sleep and can either select skip, to skip this step, or track, to enter information regarding their sleep and have their sleep tracked.

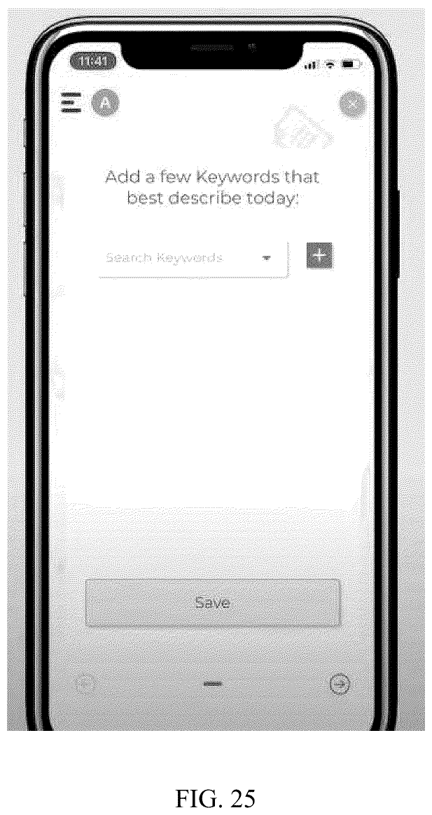

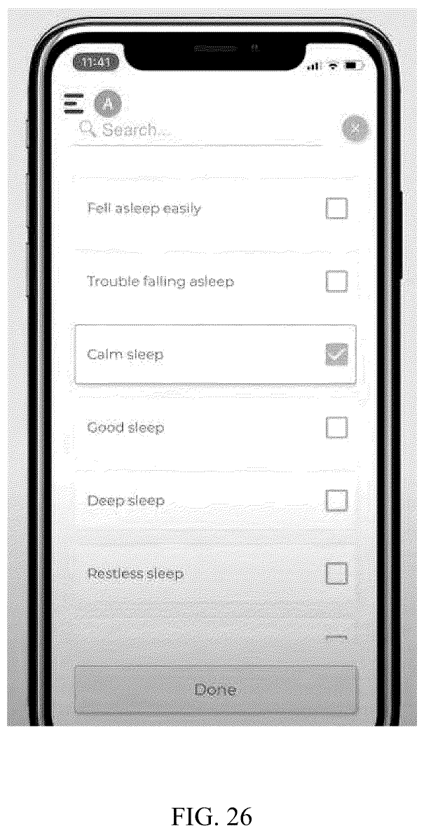

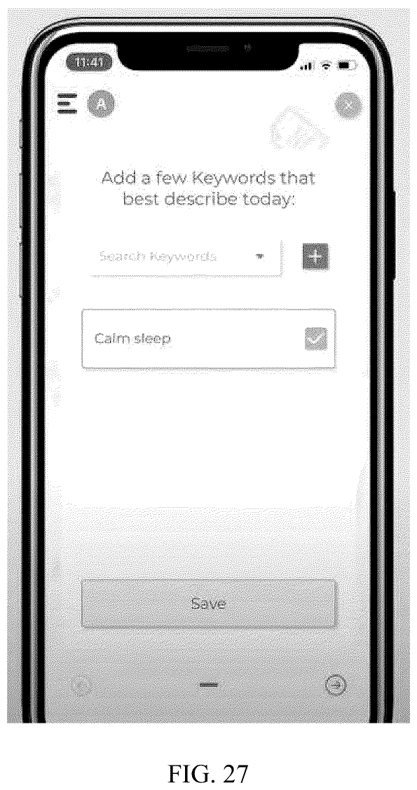

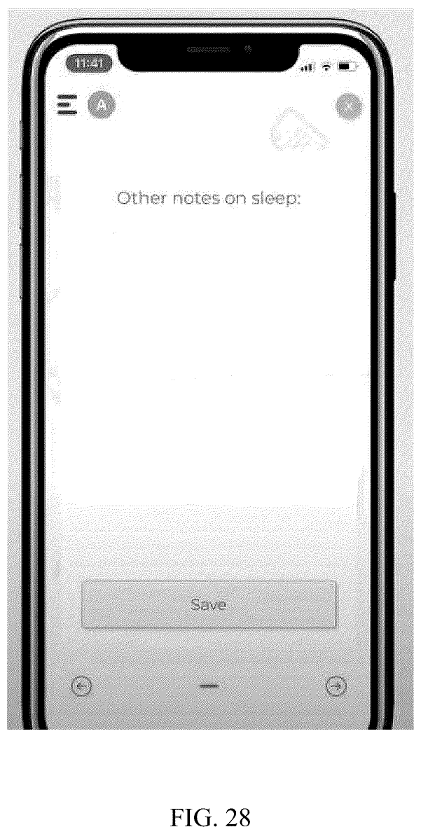

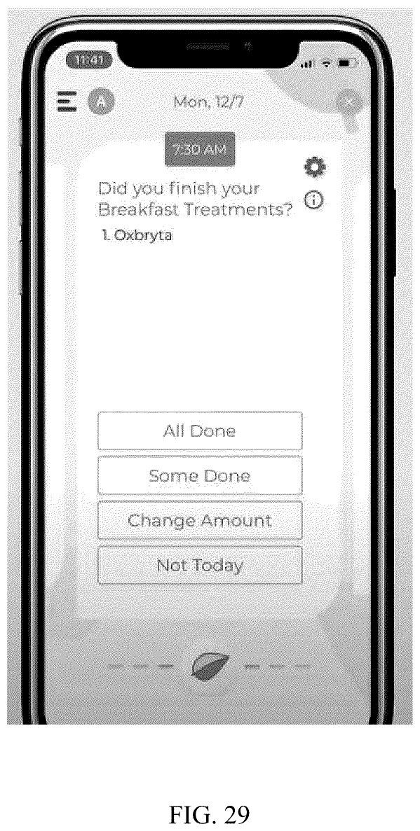

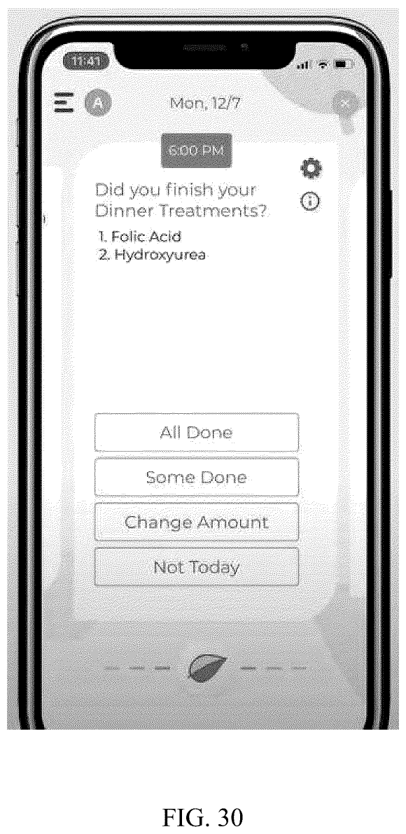

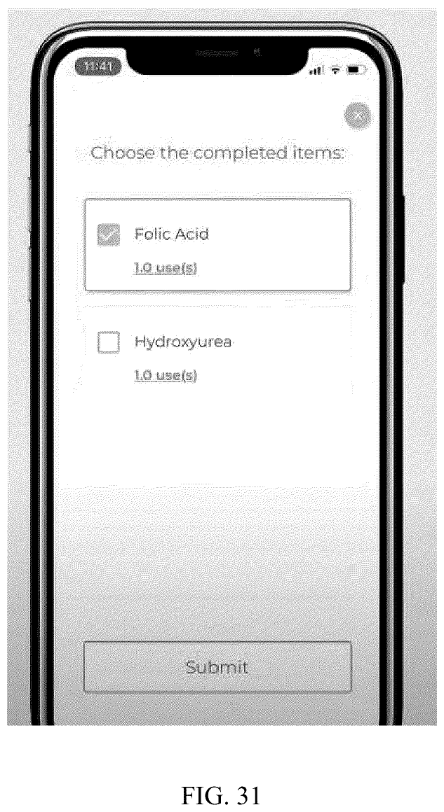

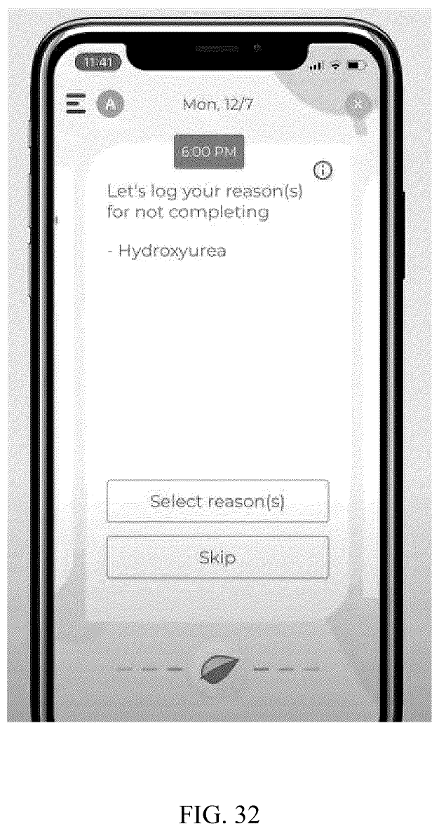

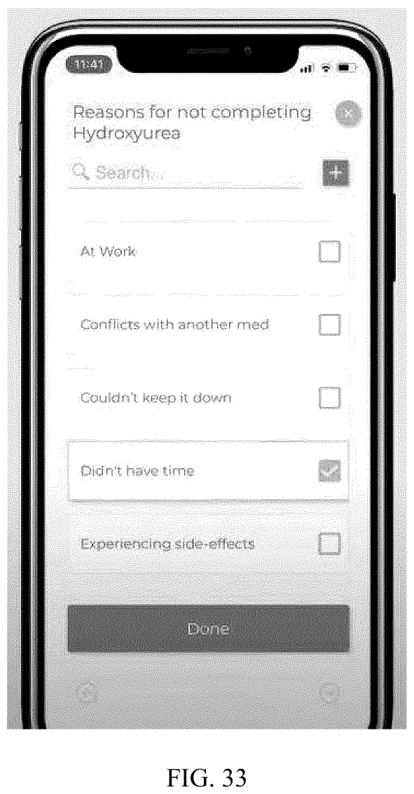

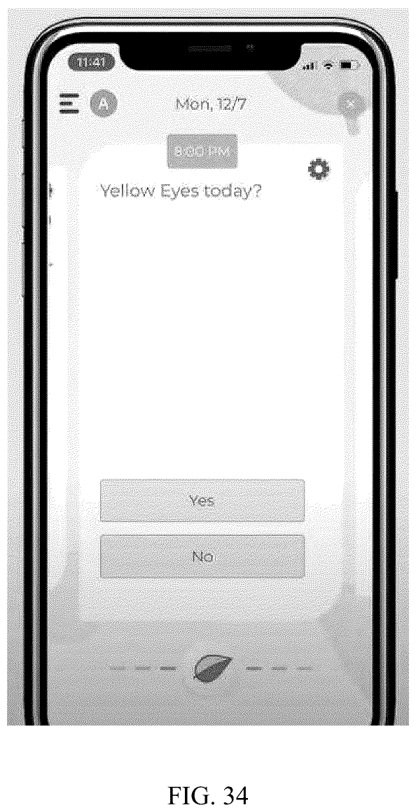

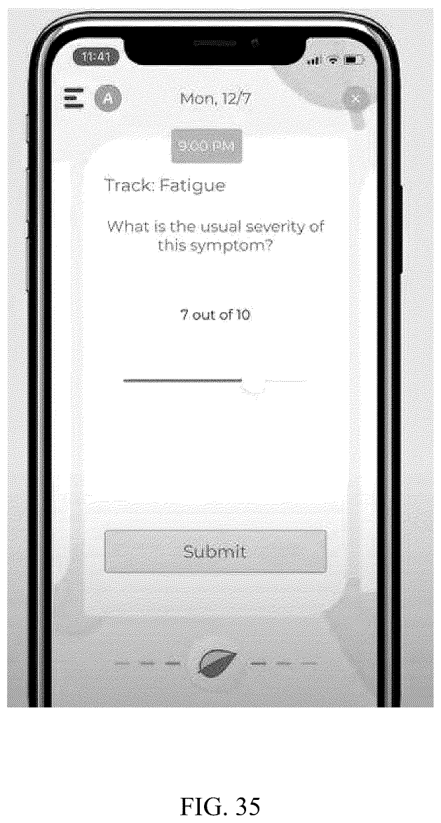

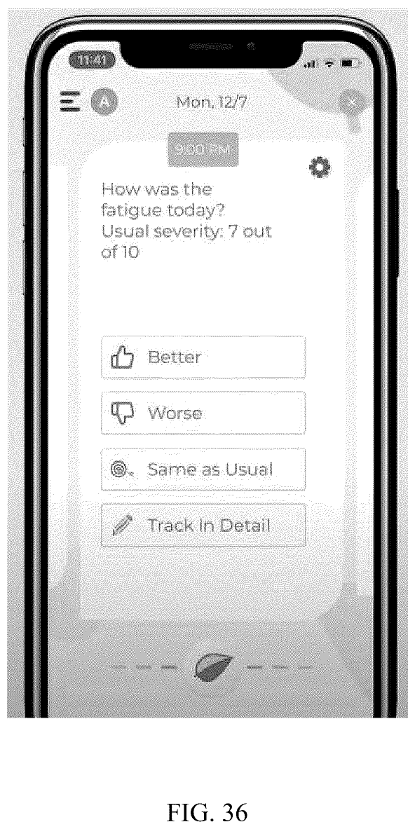

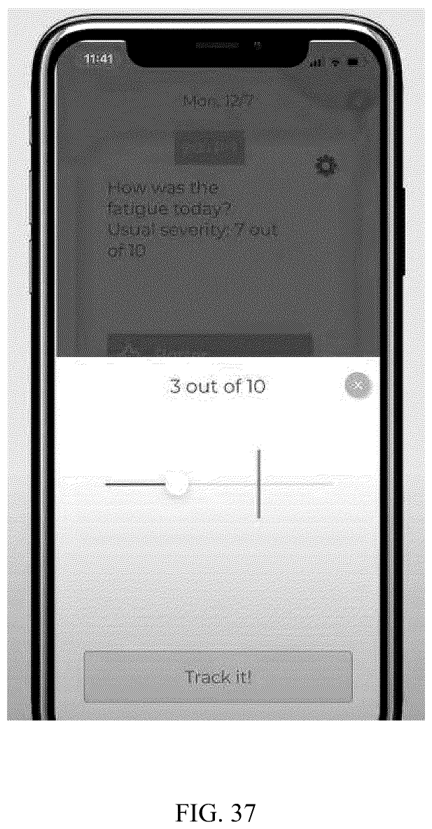

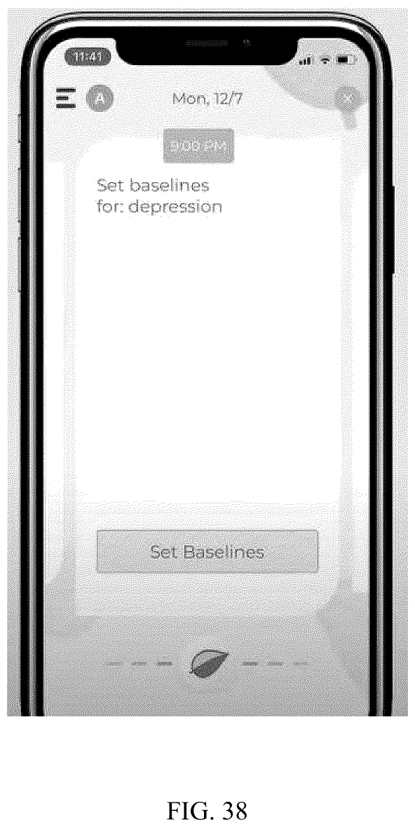

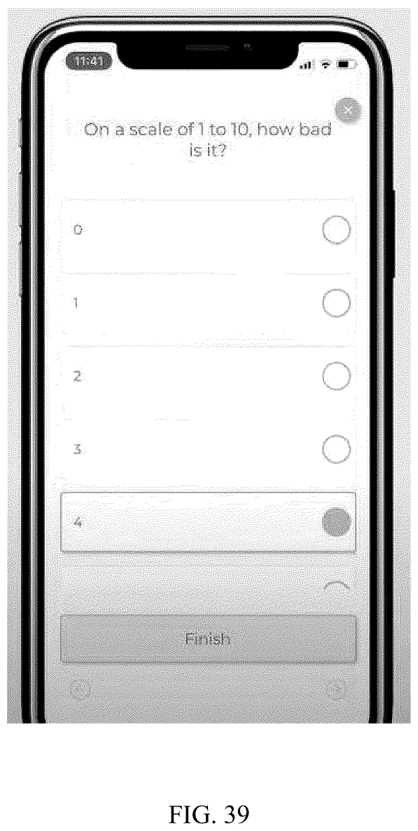

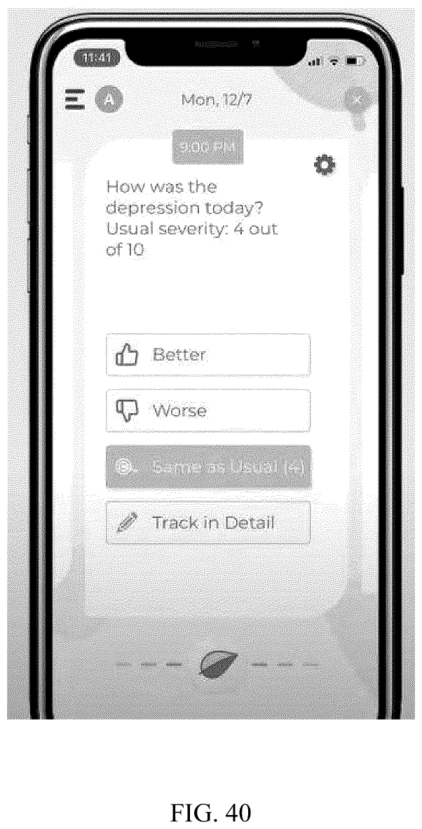

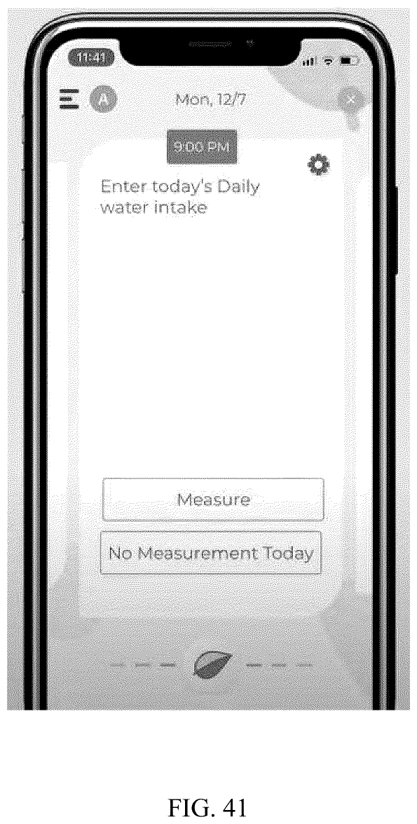

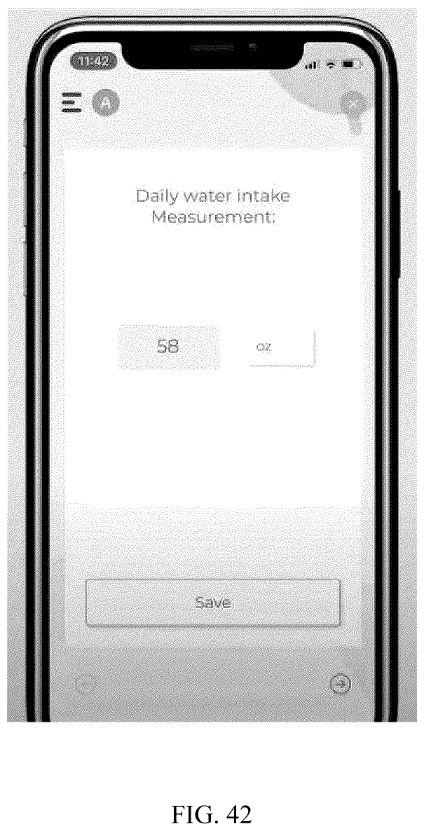

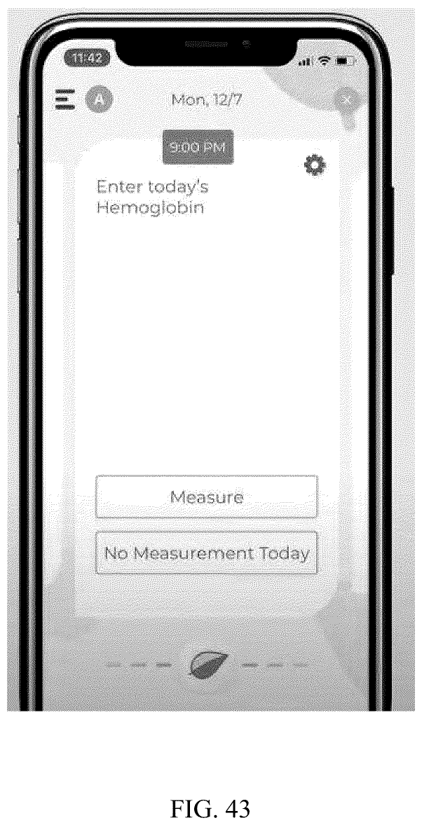

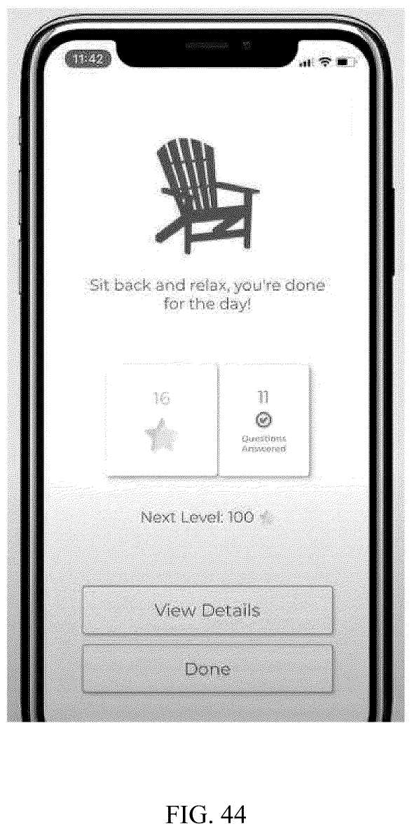

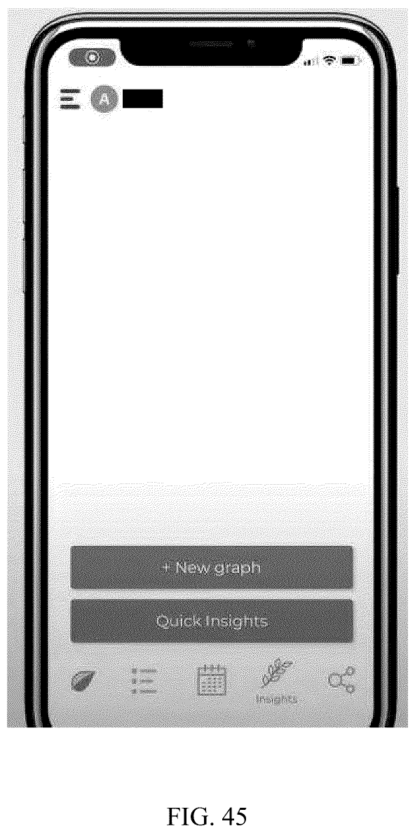

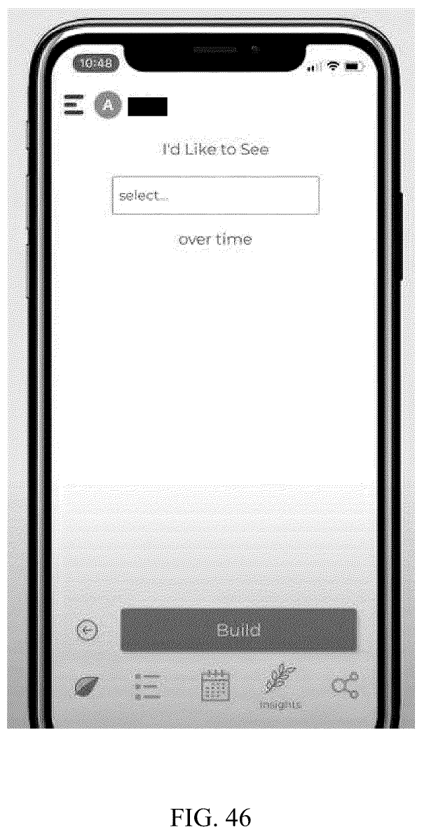

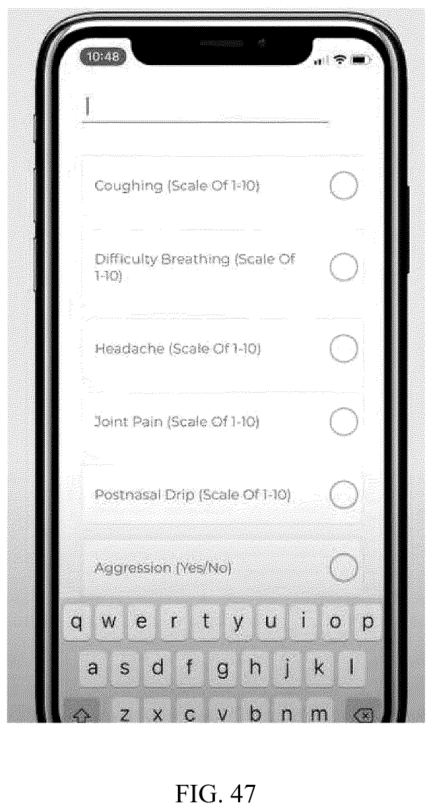

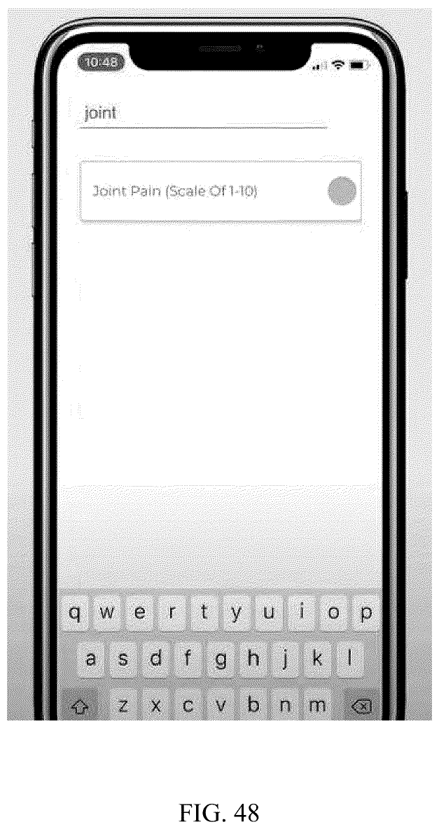

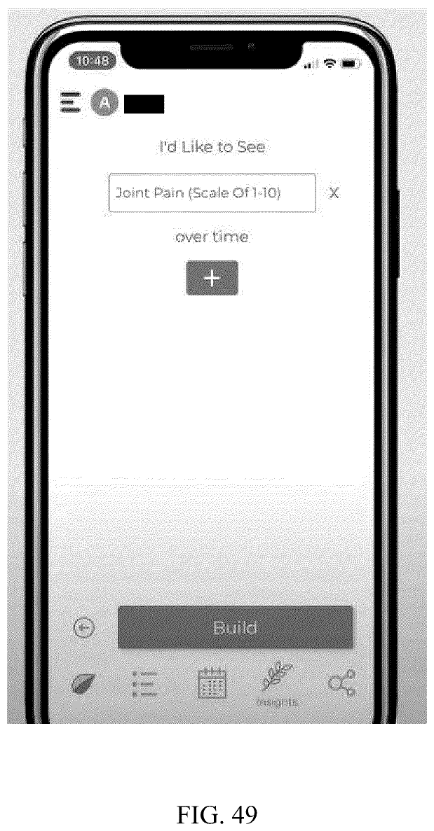

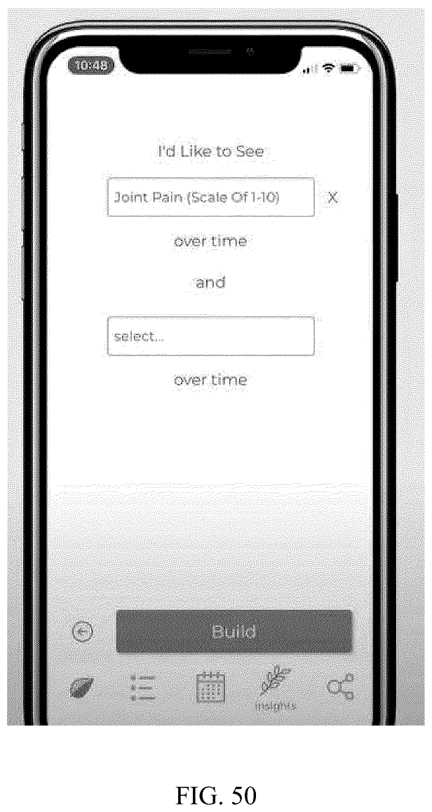

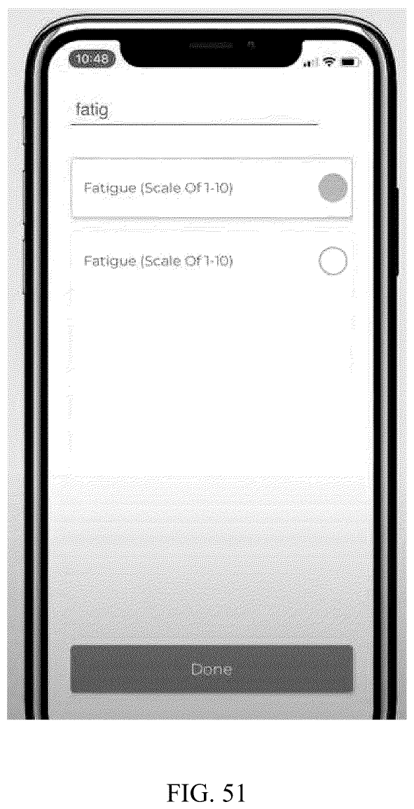

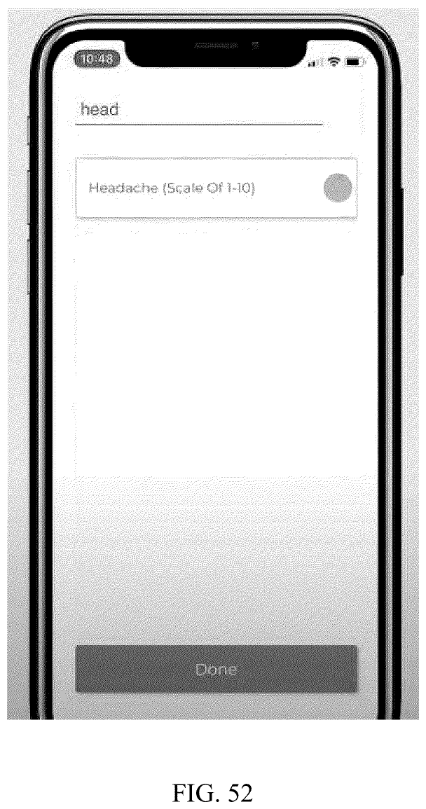

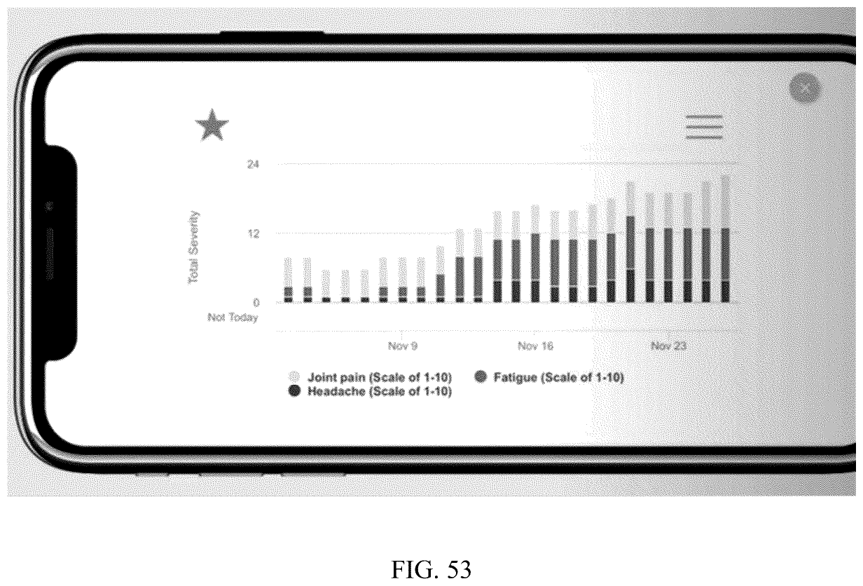

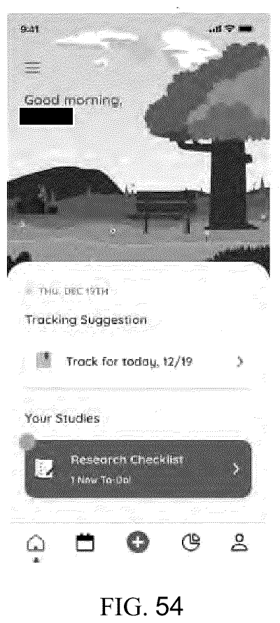

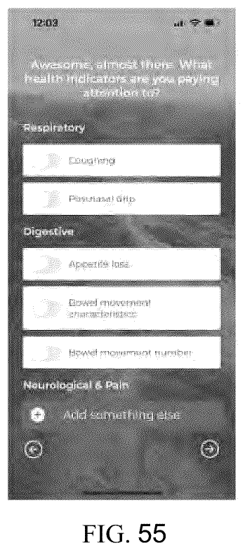

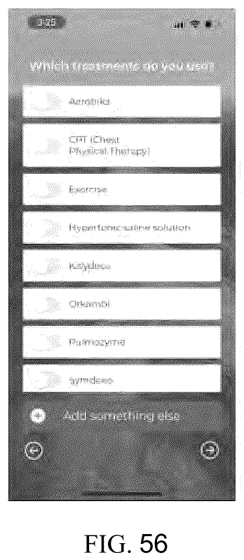

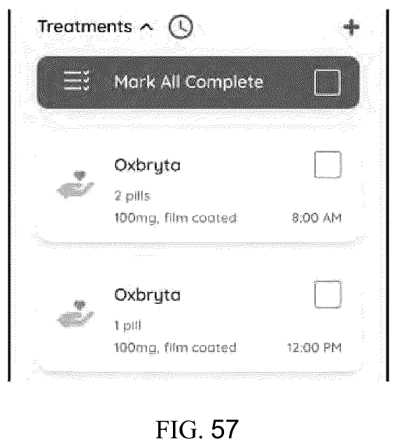

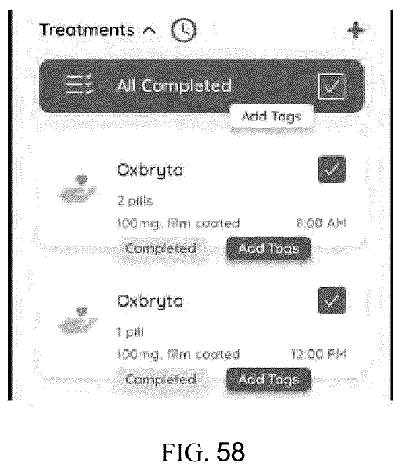

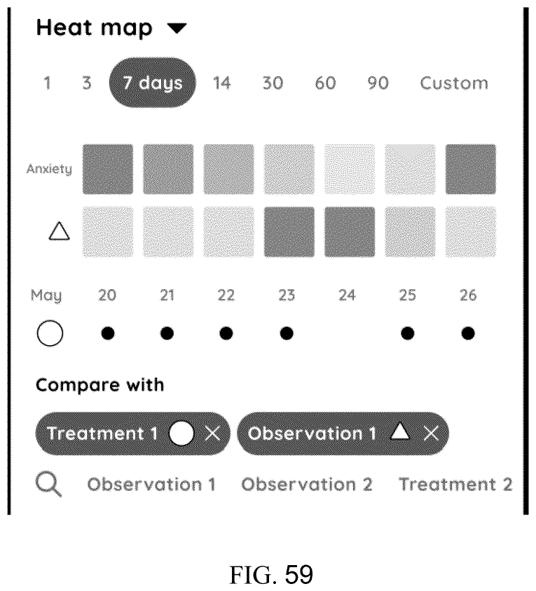

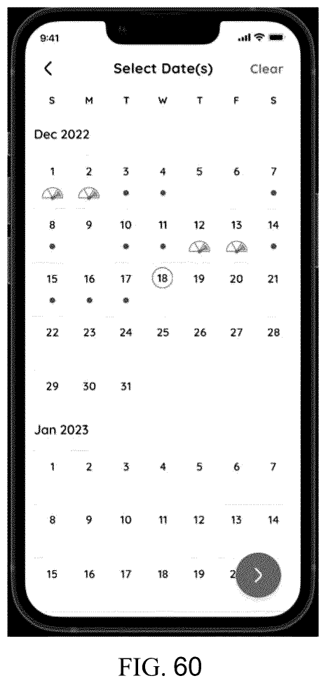

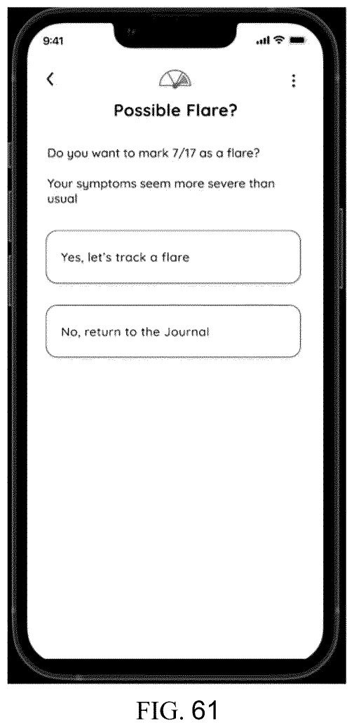

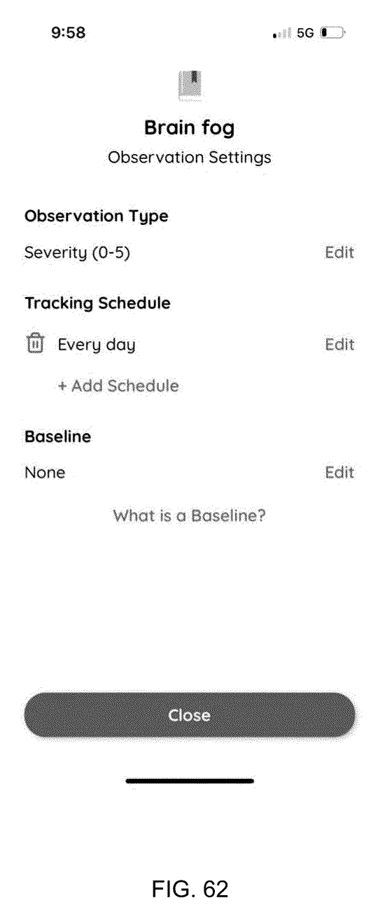

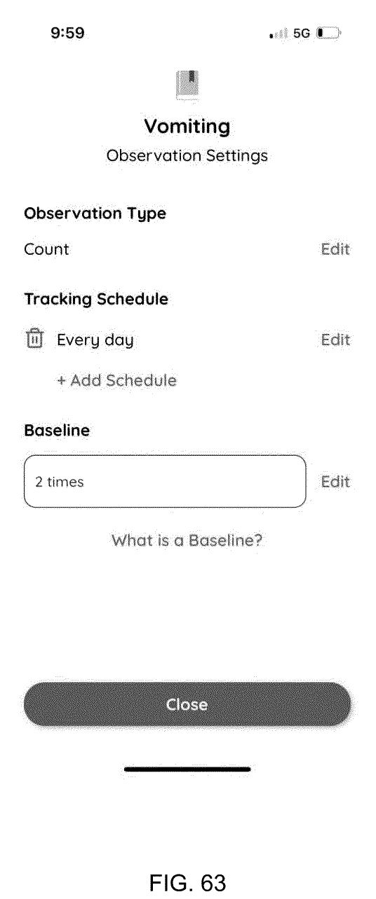

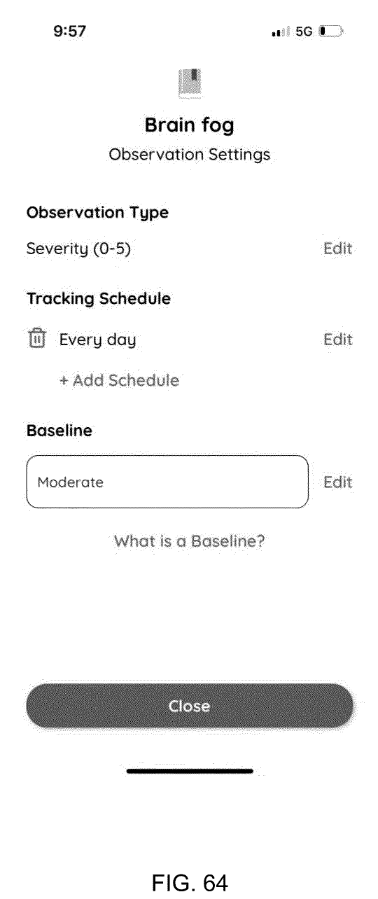

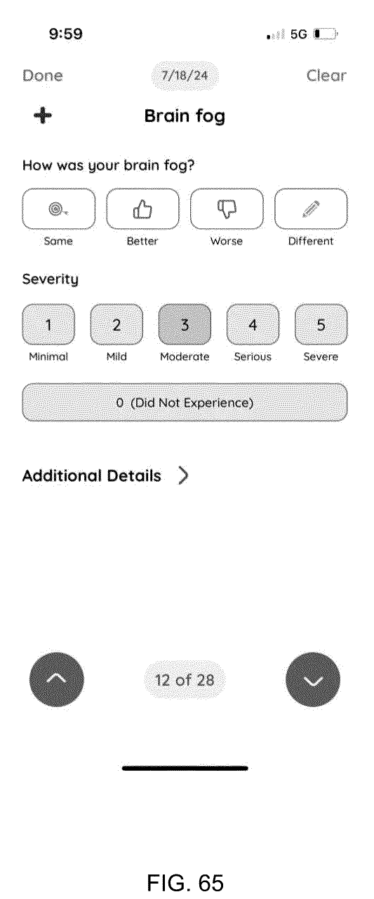

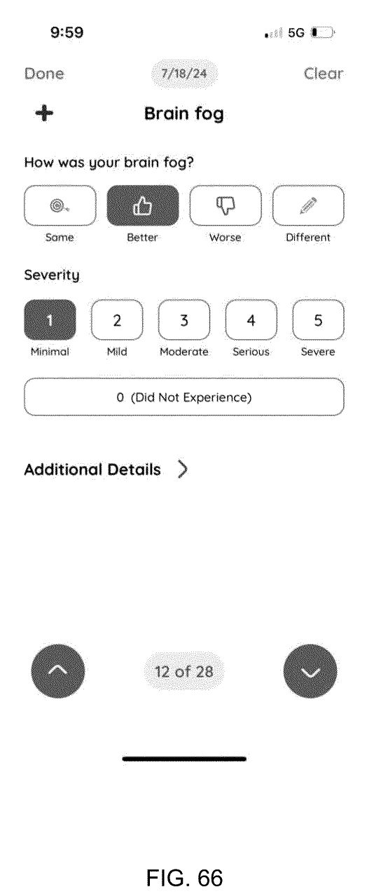

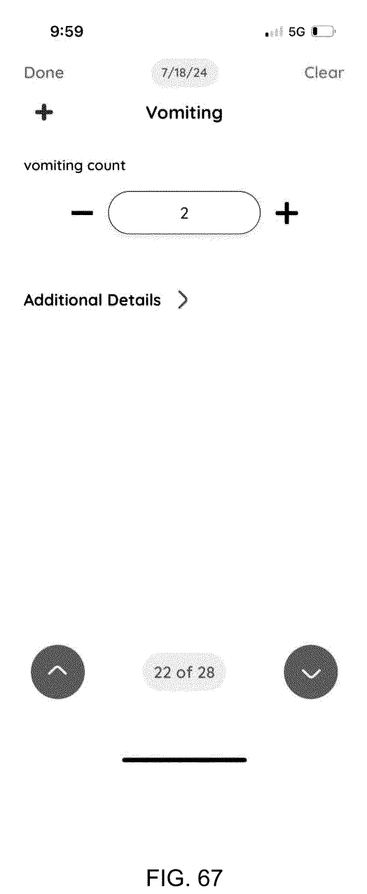

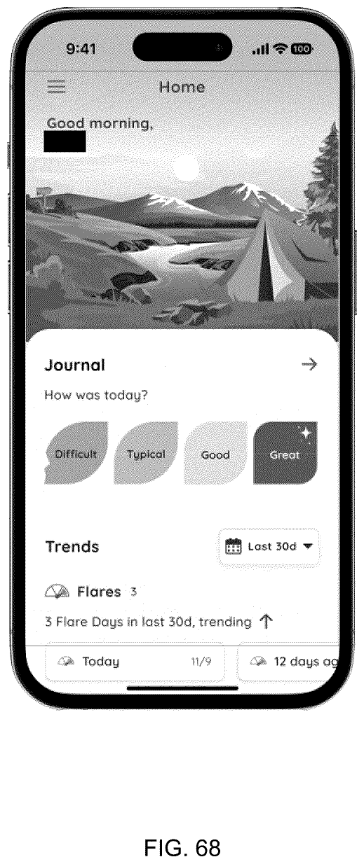

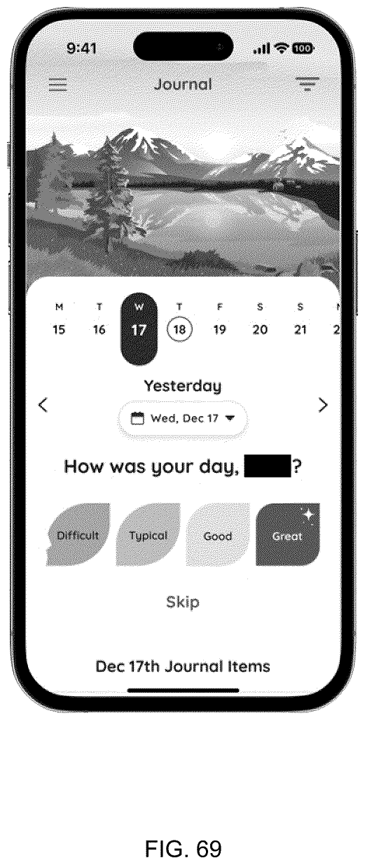

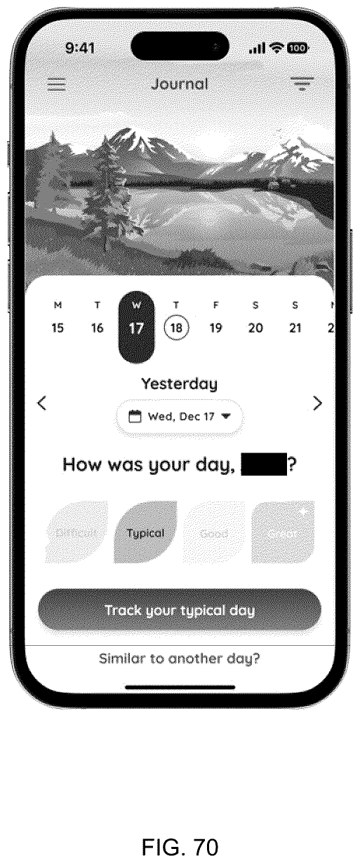

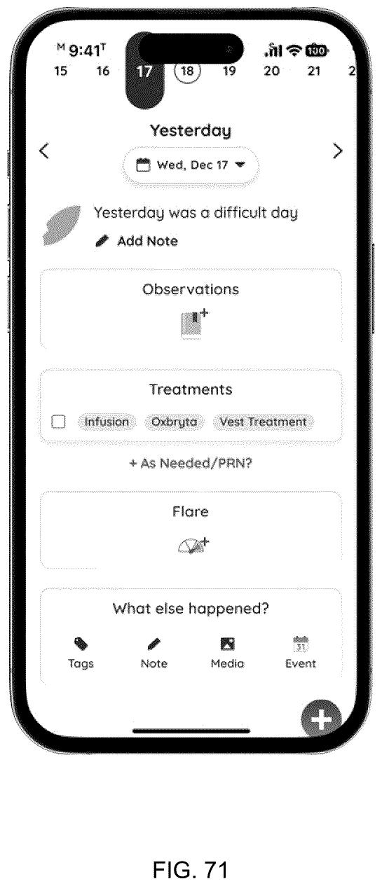

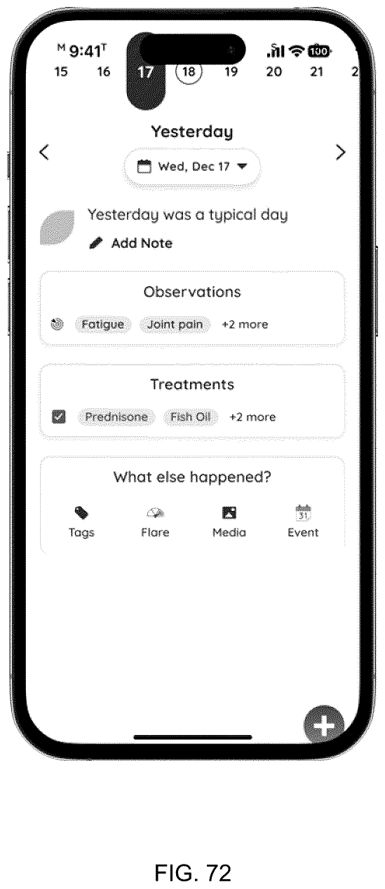

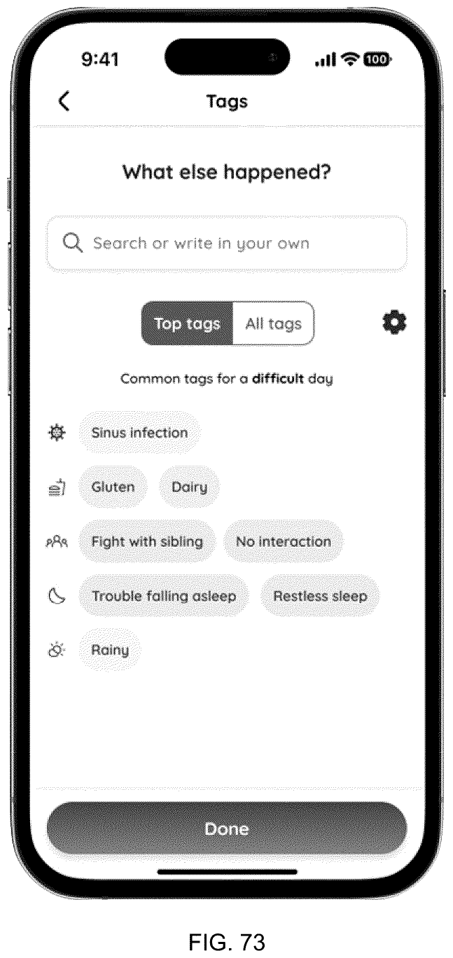

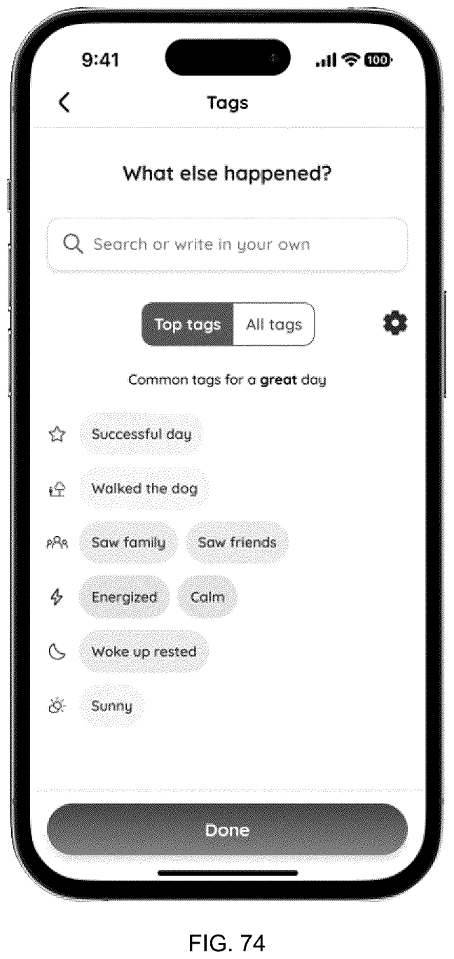

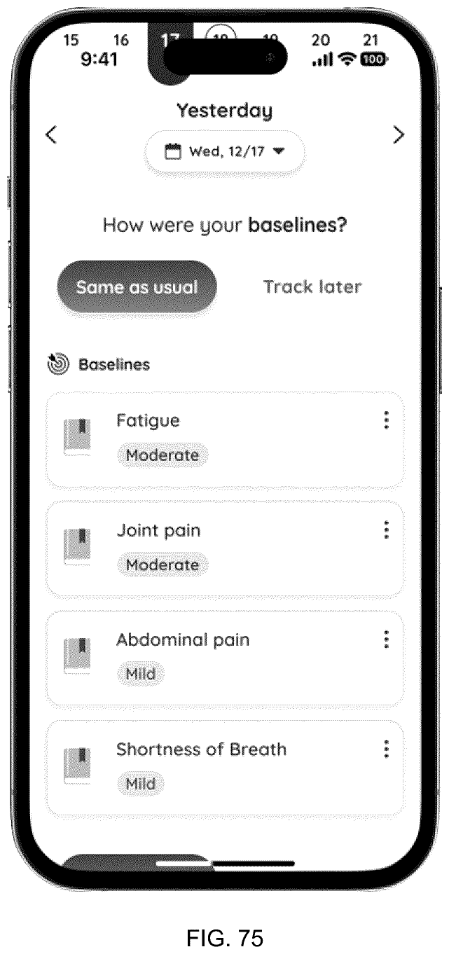

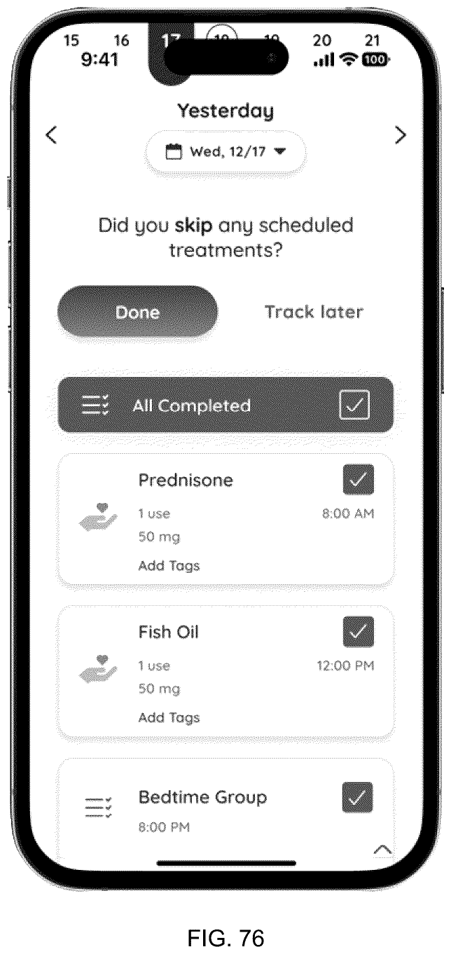

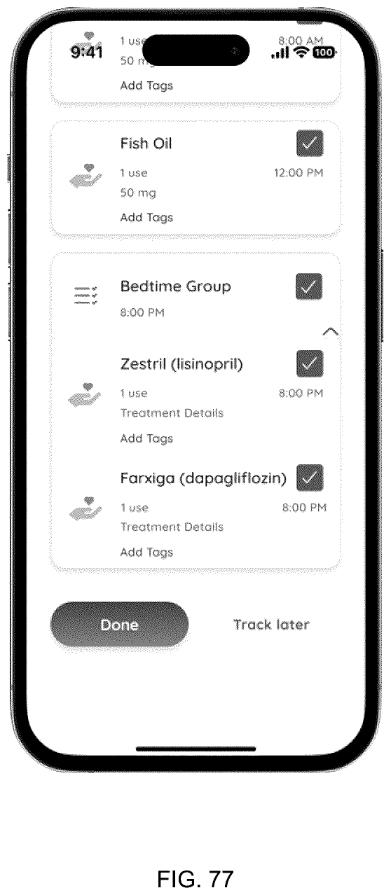

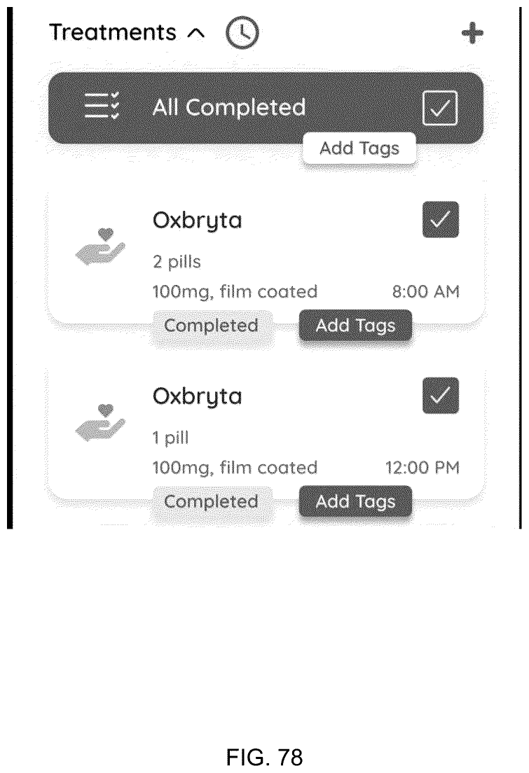

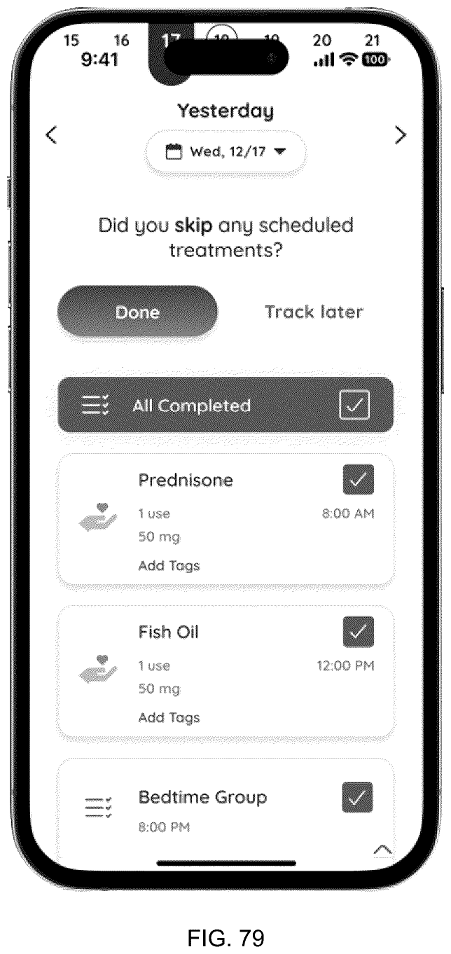

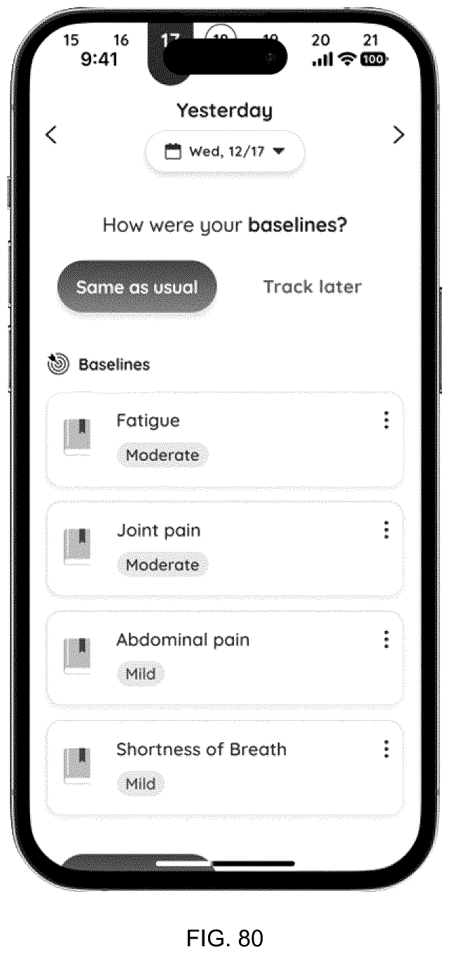

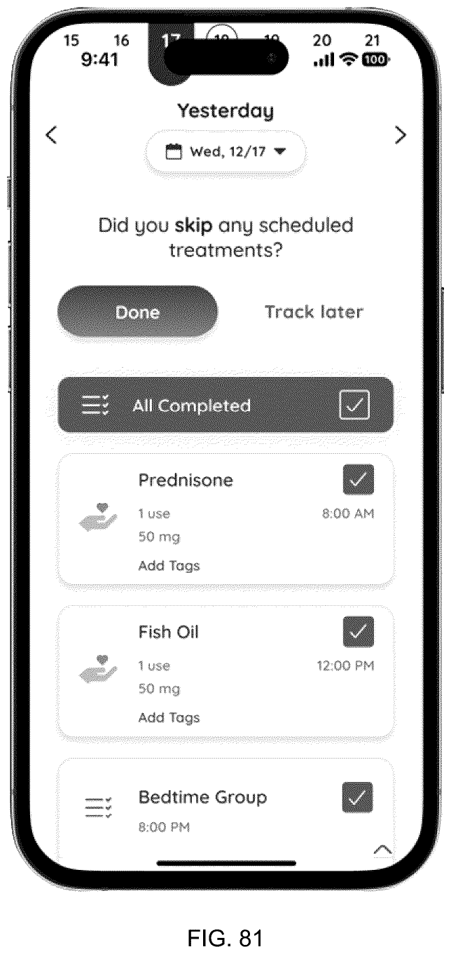

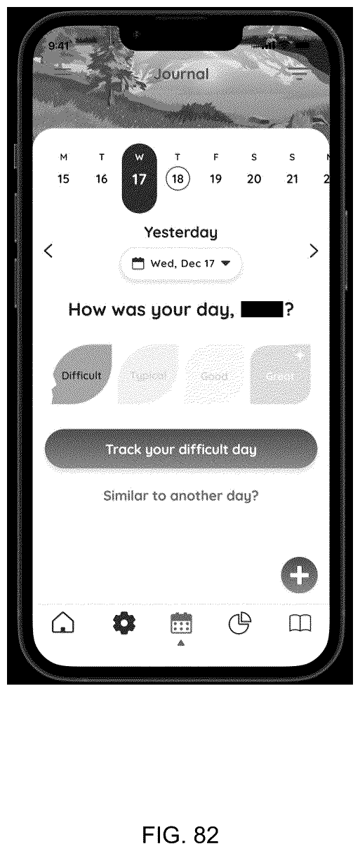

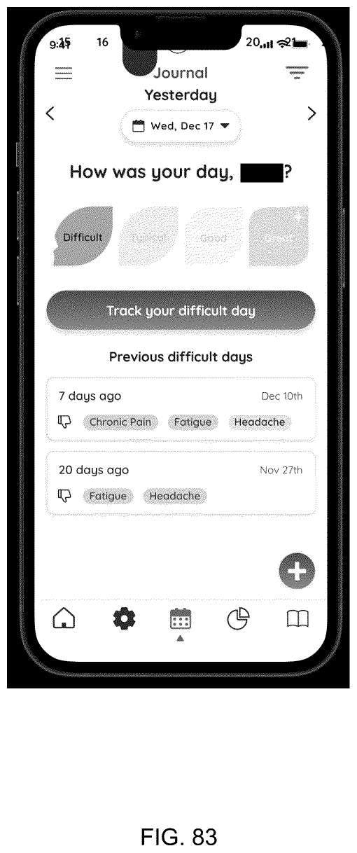

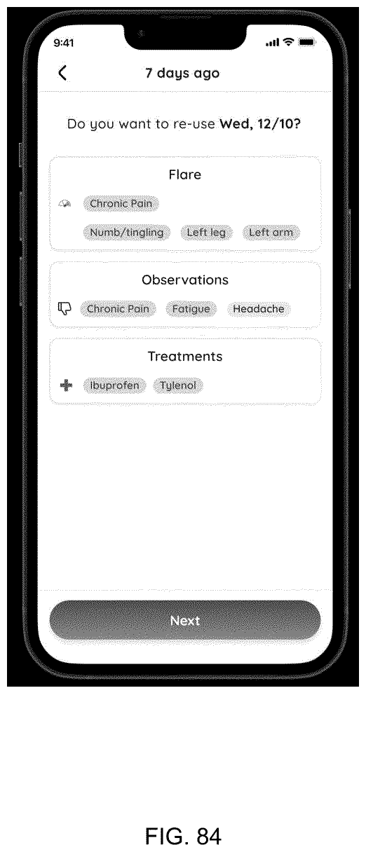

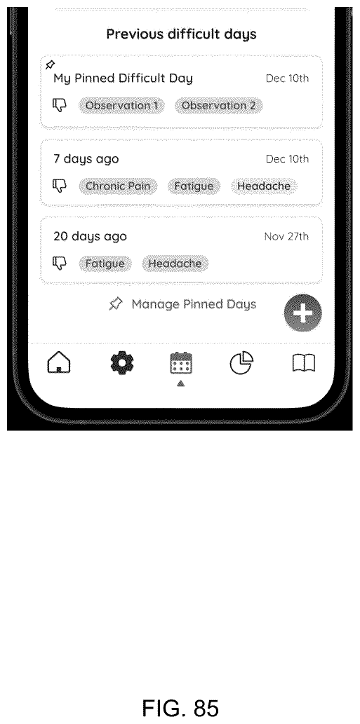

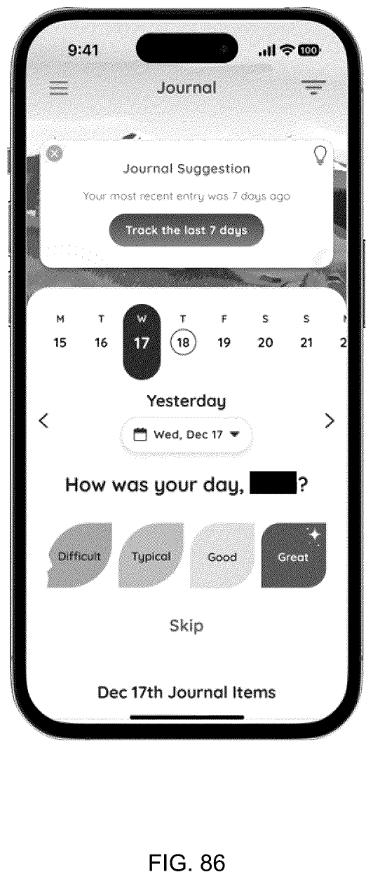

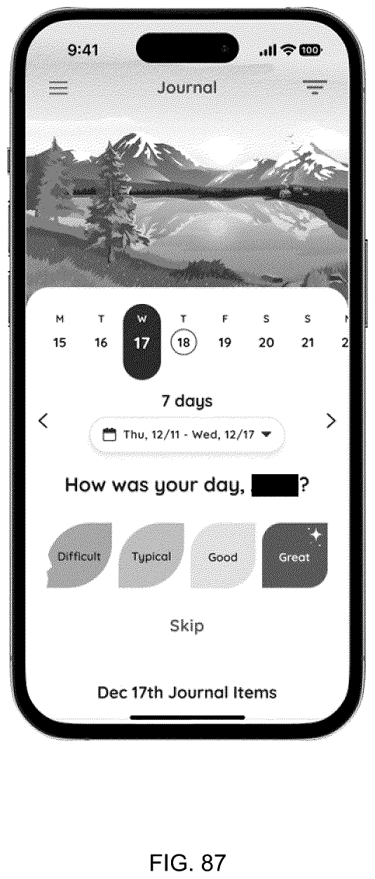

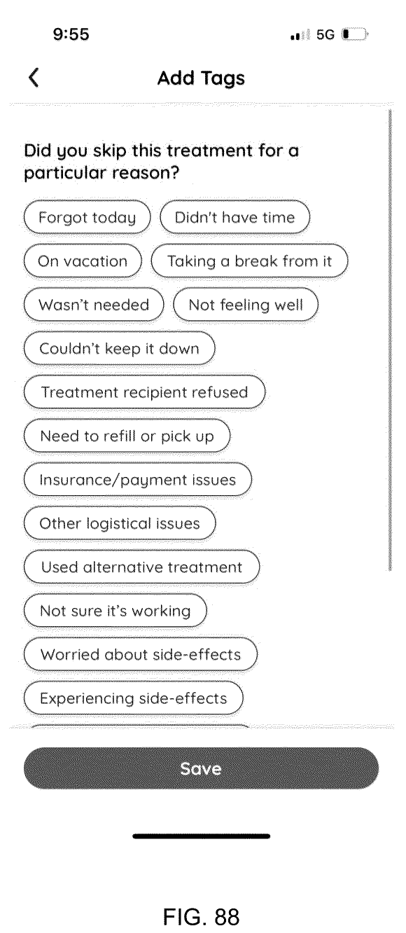

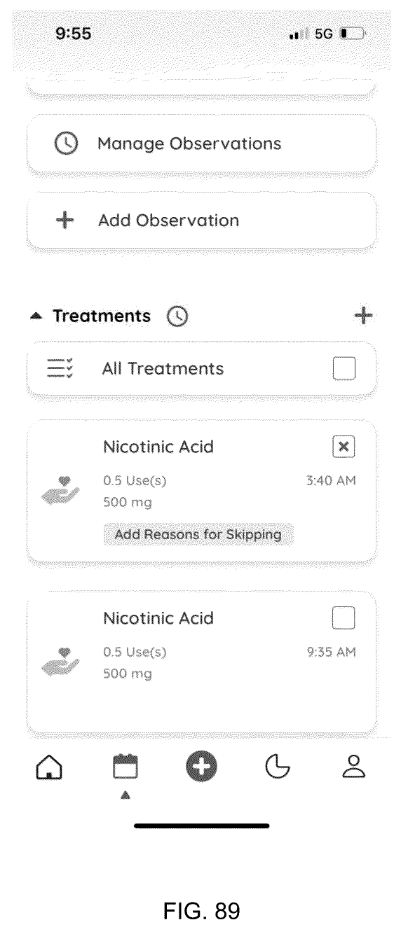

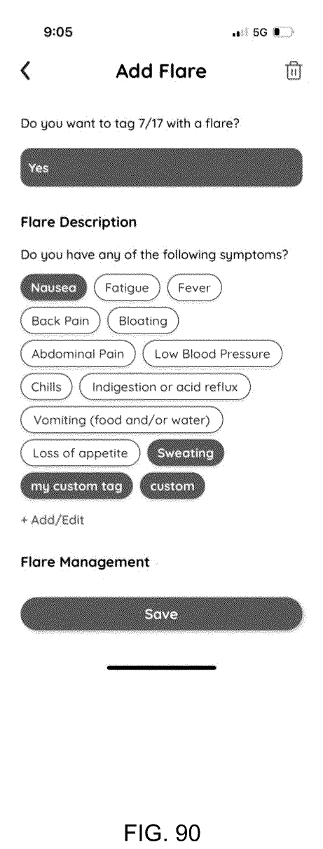

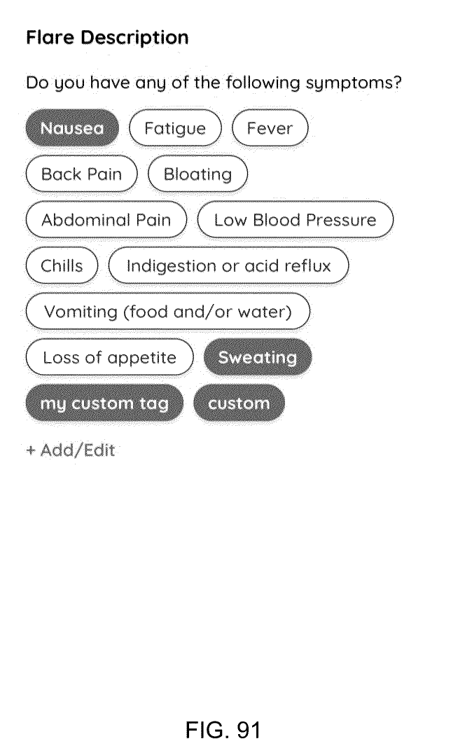

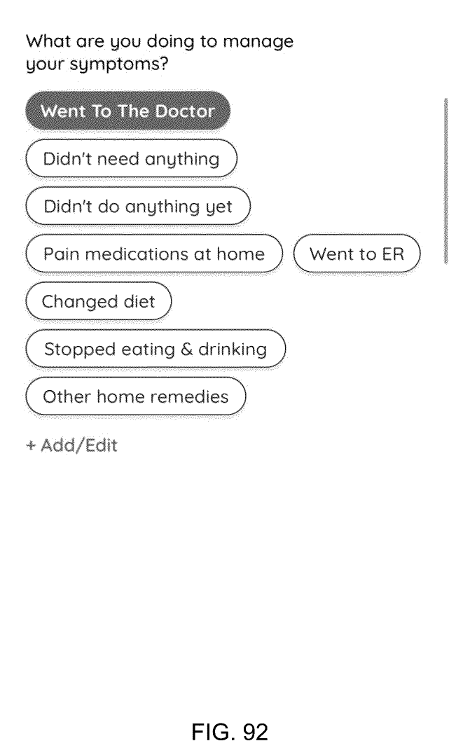

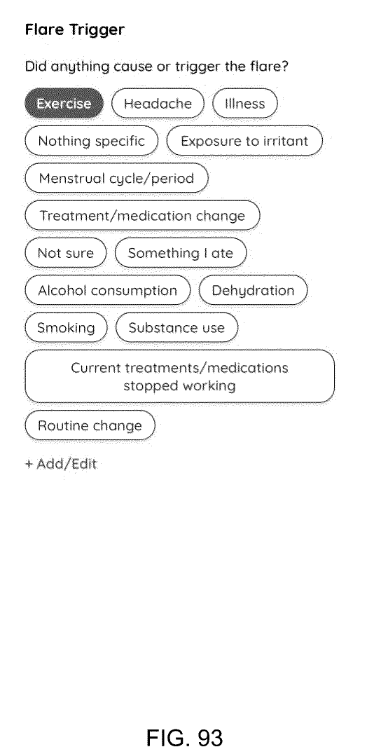

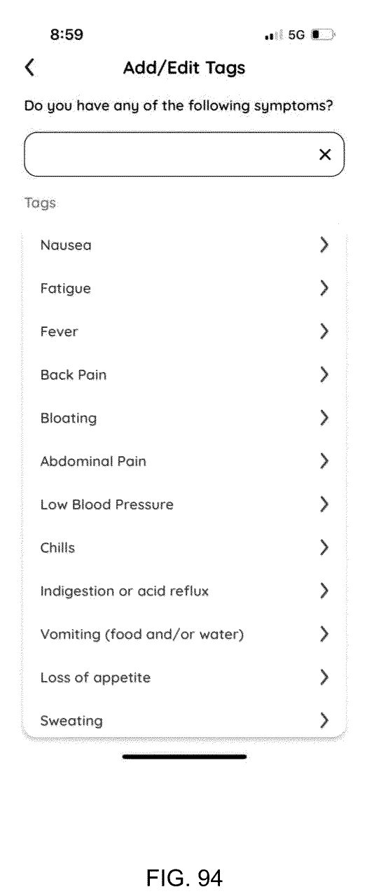

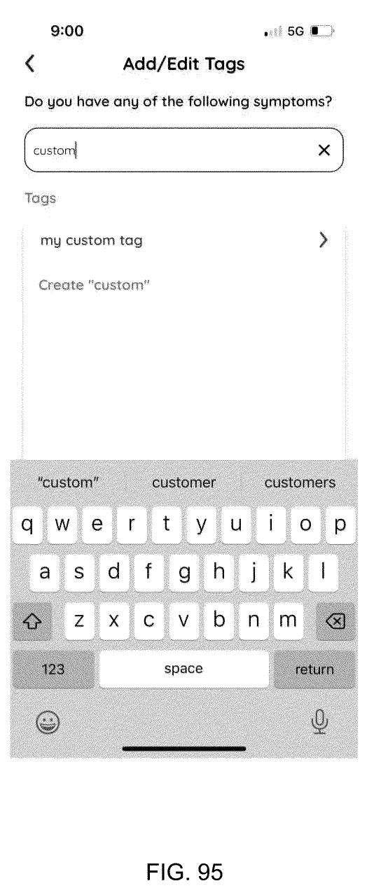

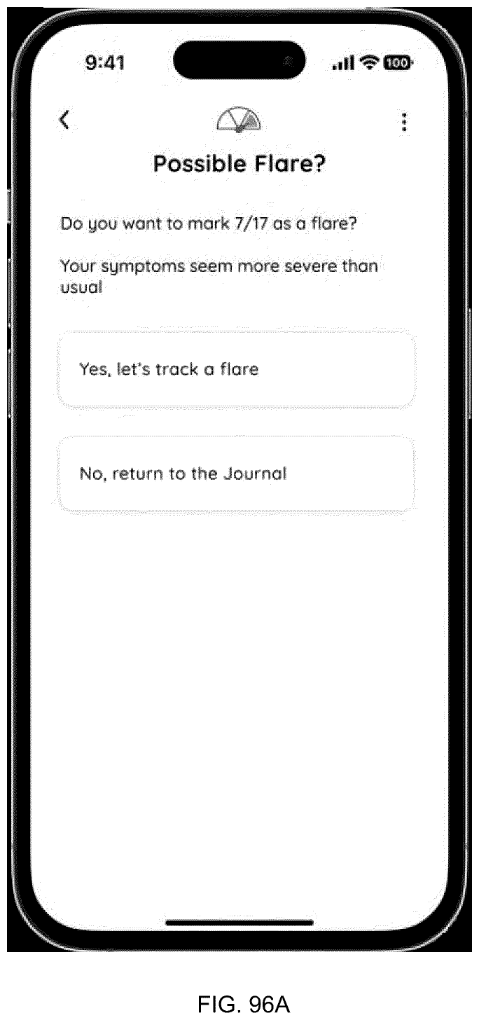

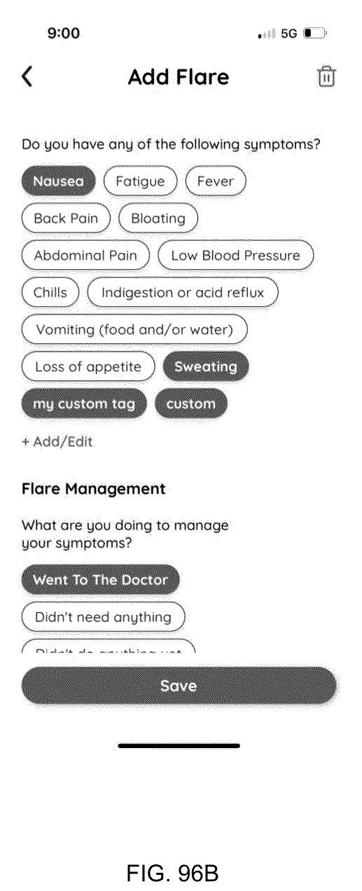

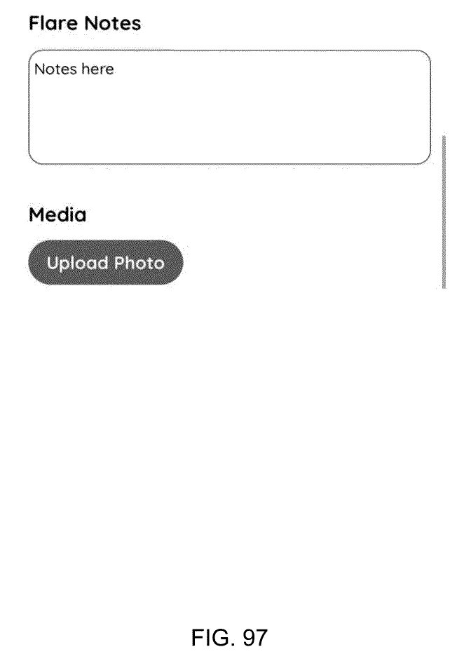

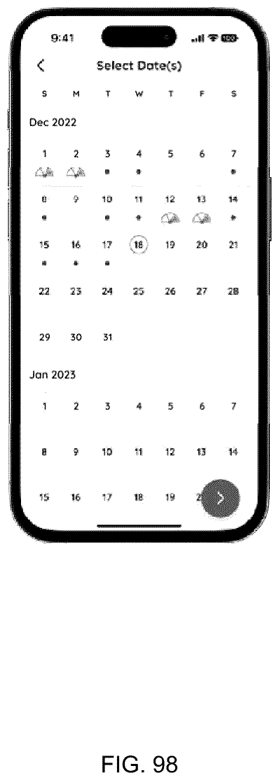

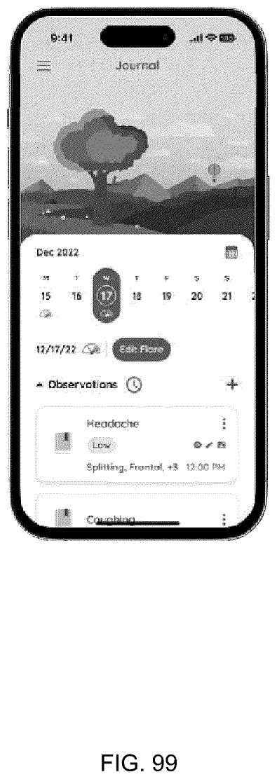

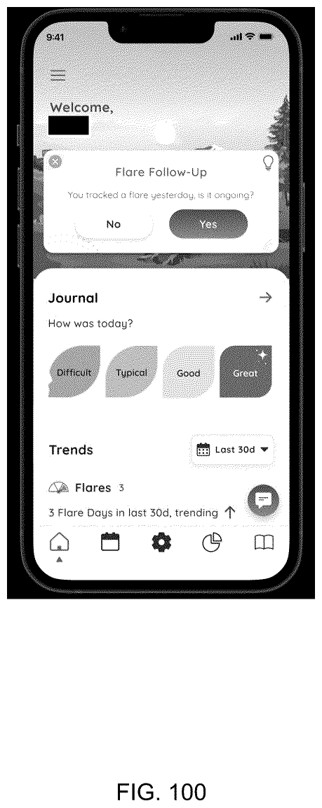

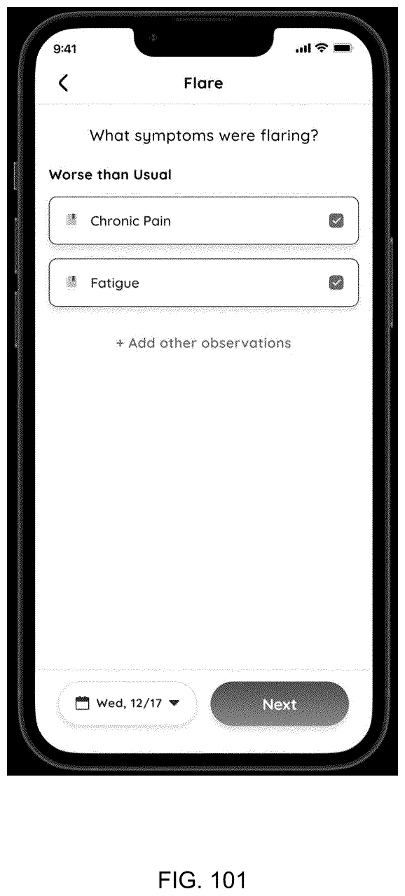

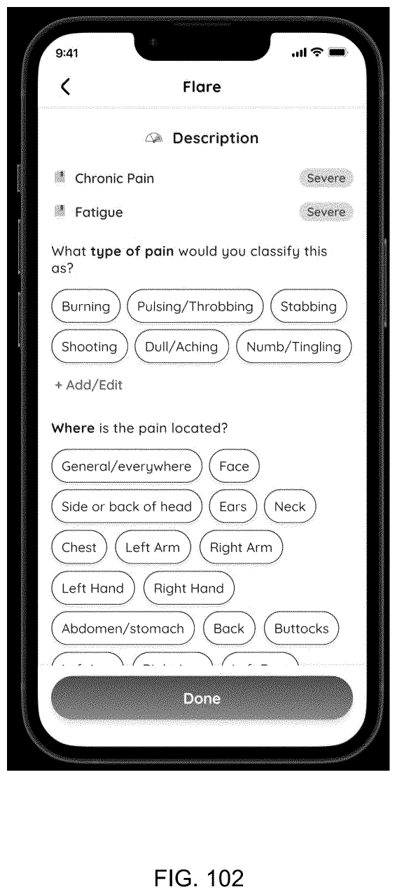

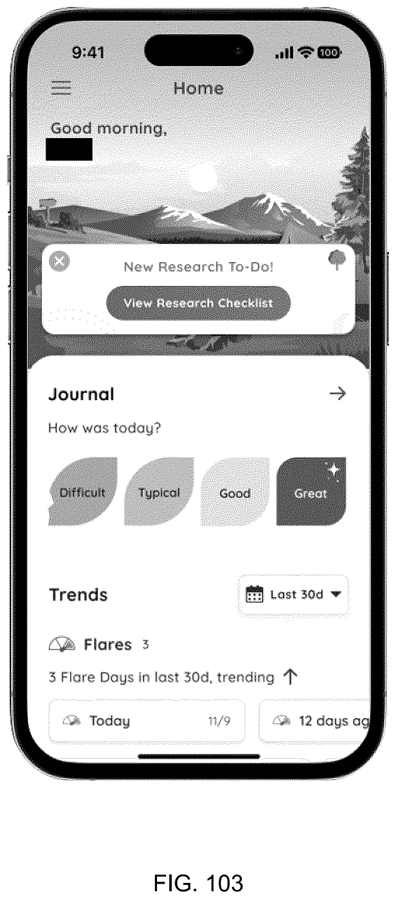

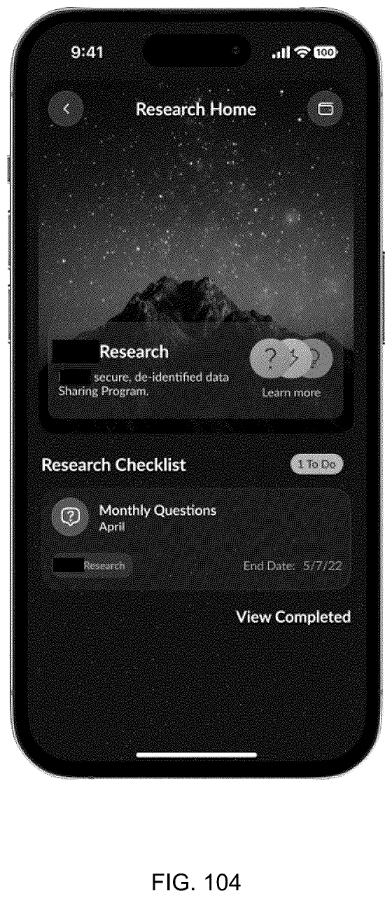

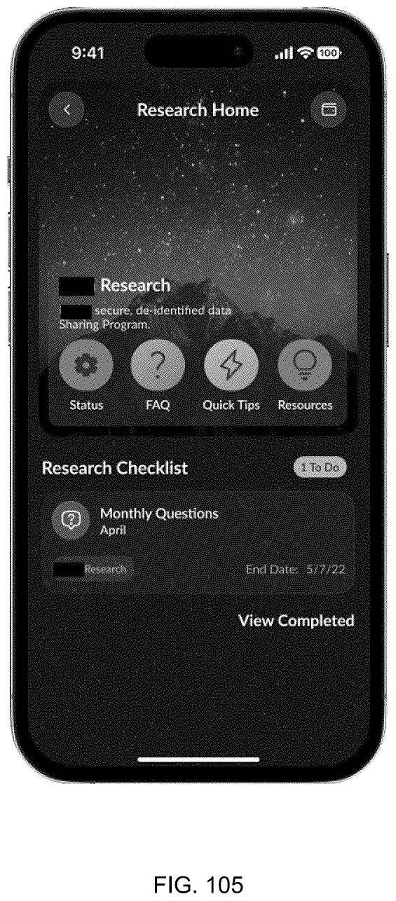

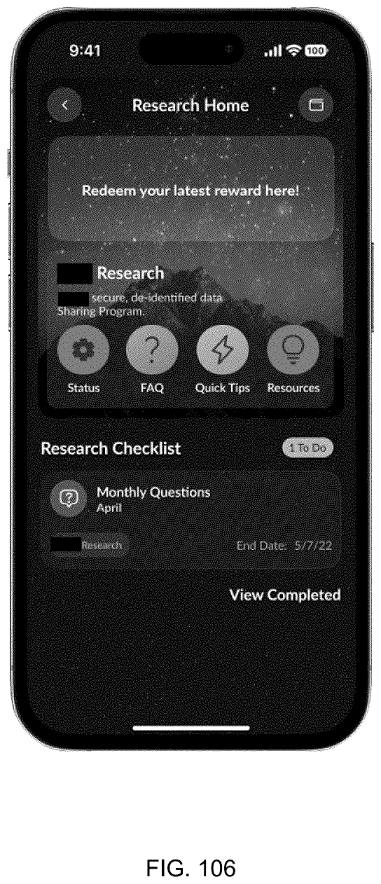

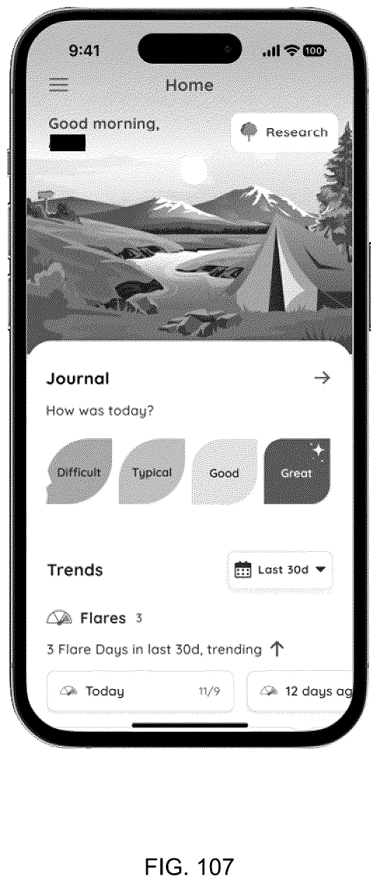

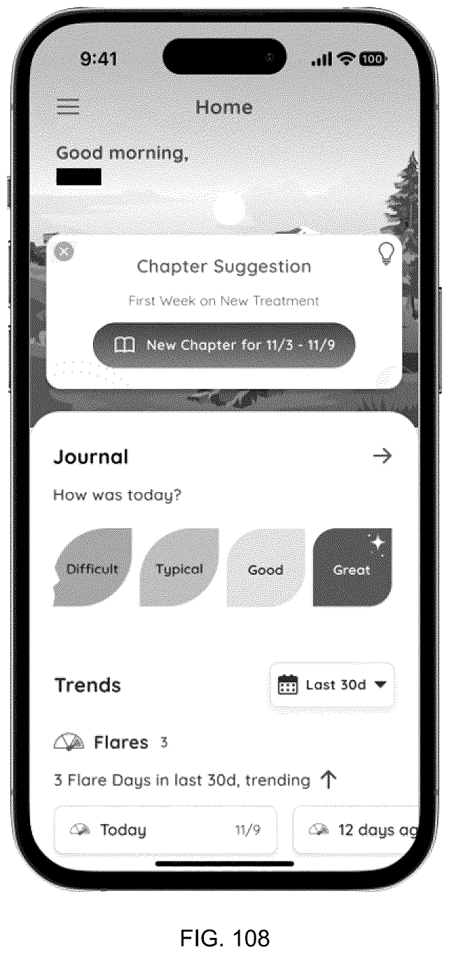

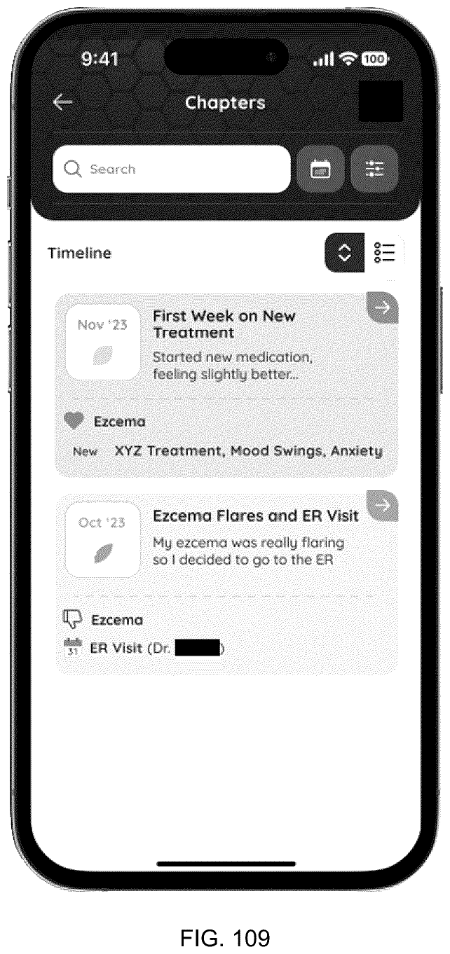

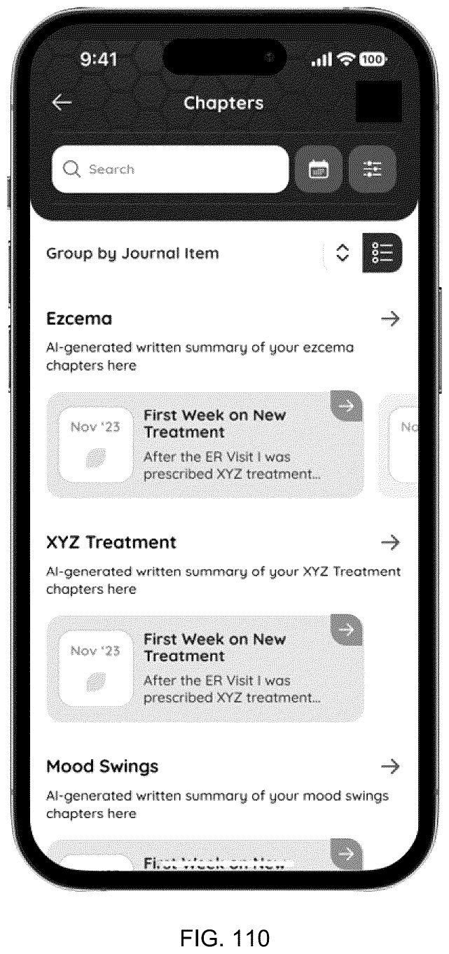

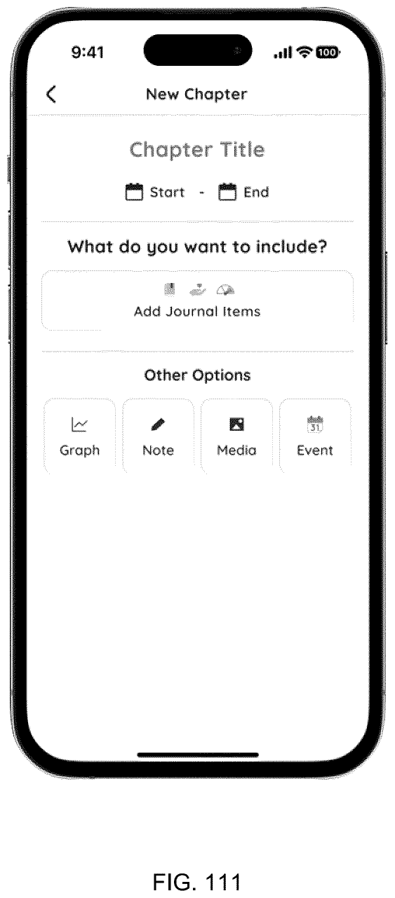

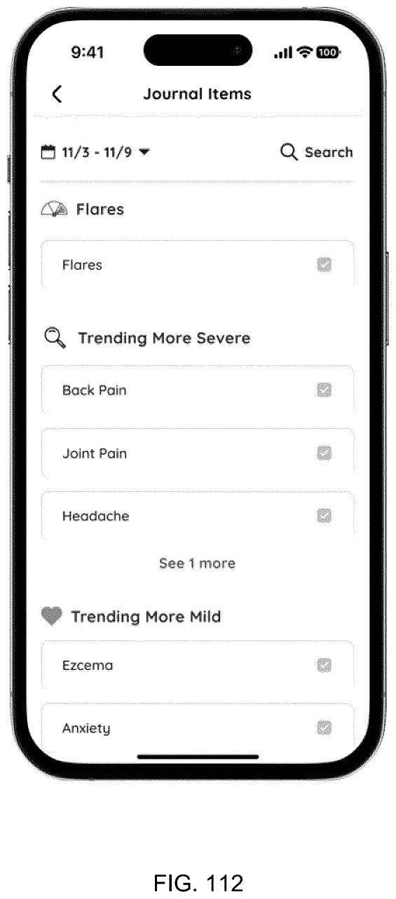

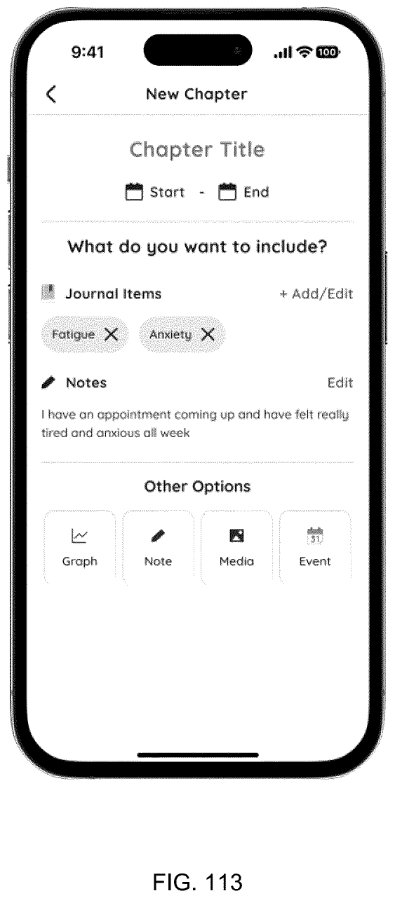

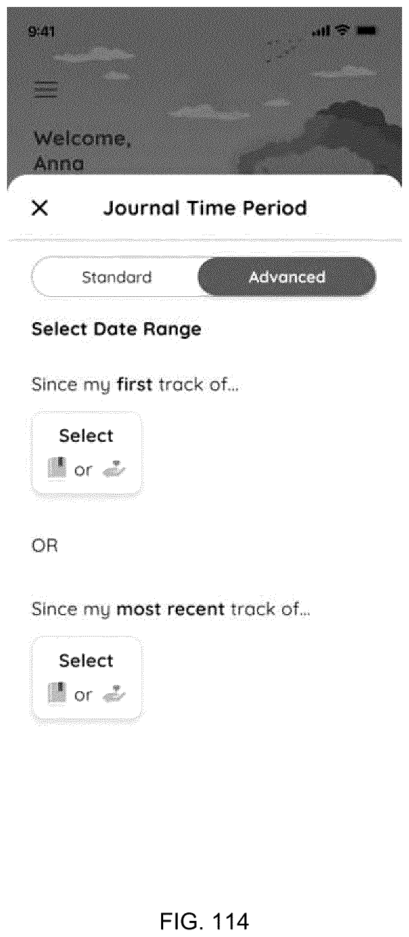

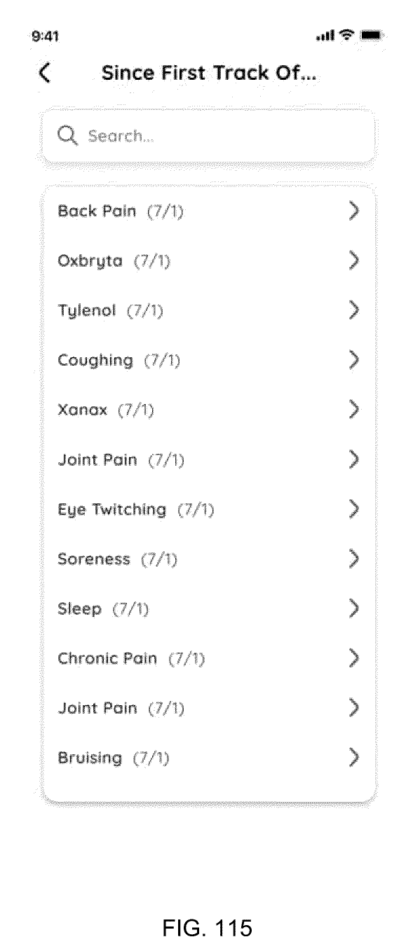

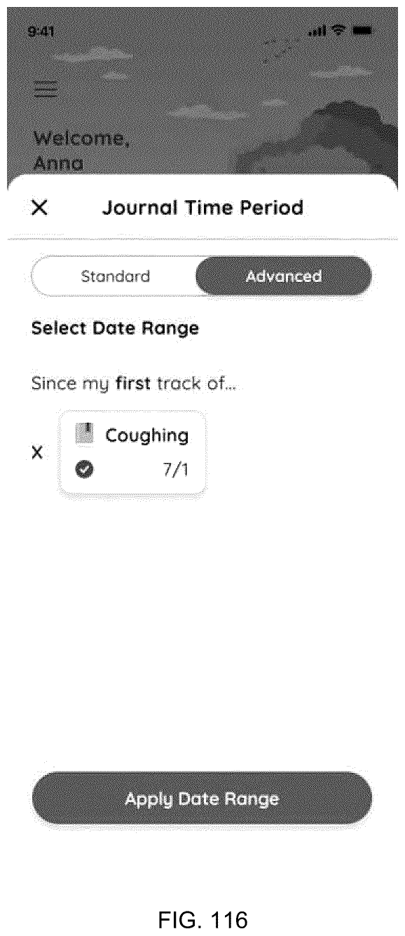

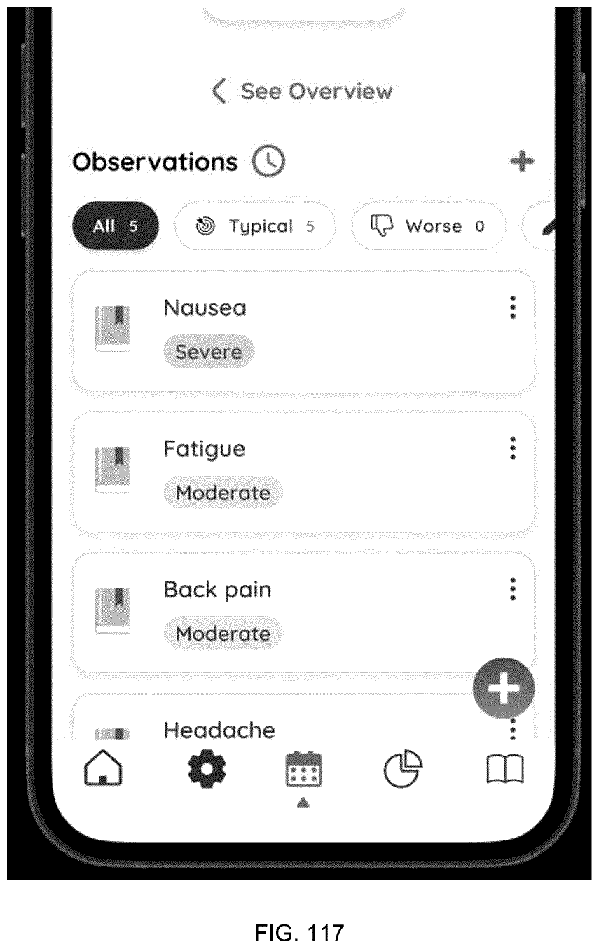

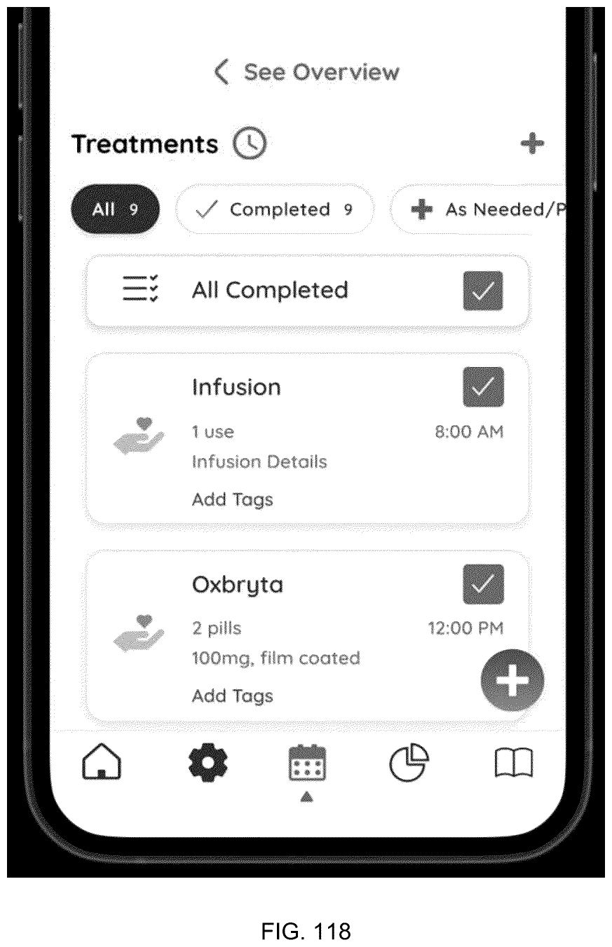

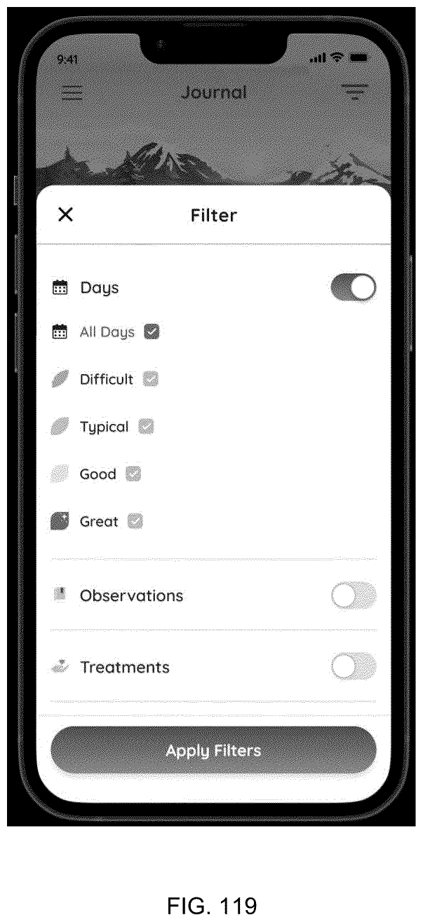

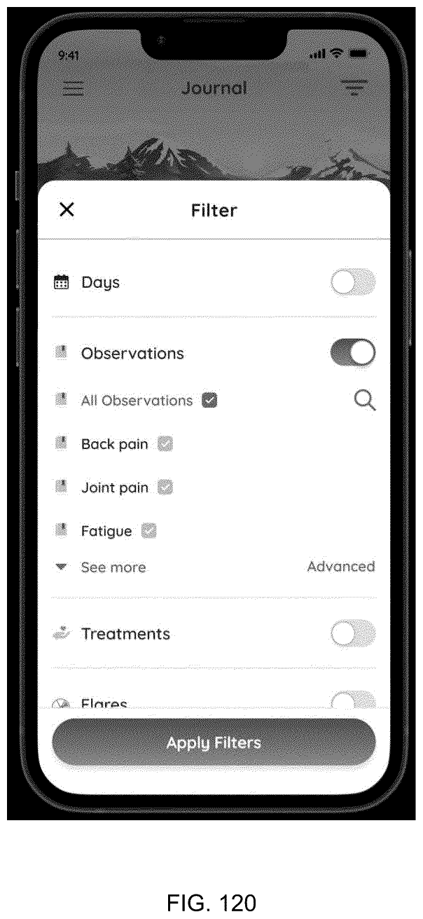

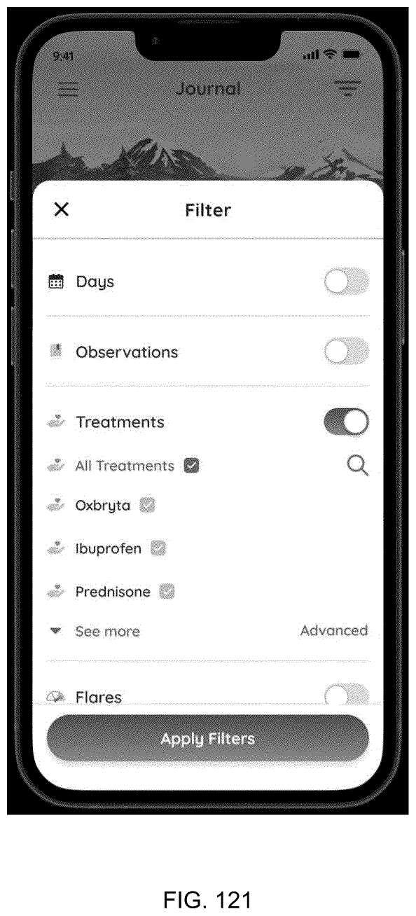

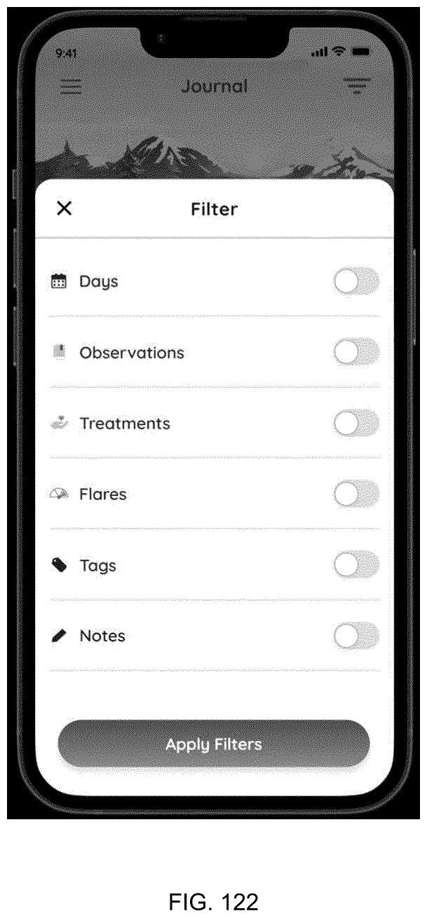

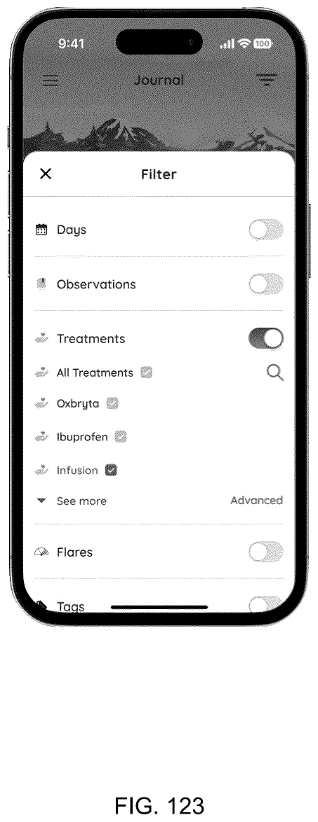

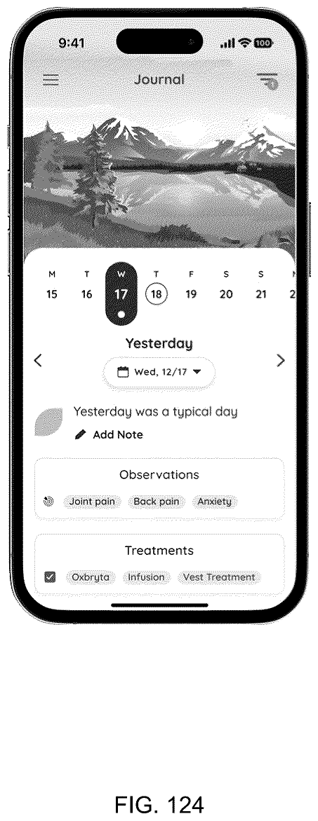

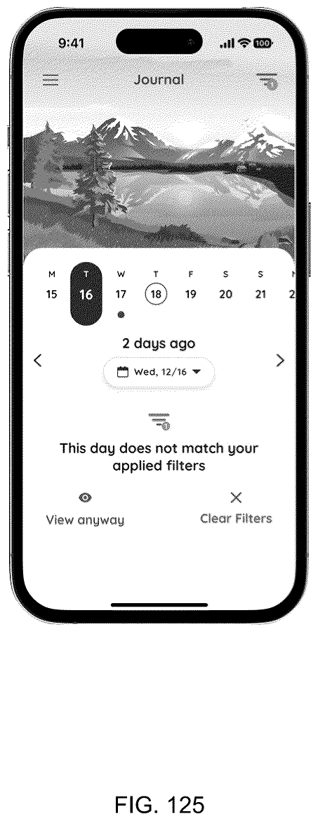

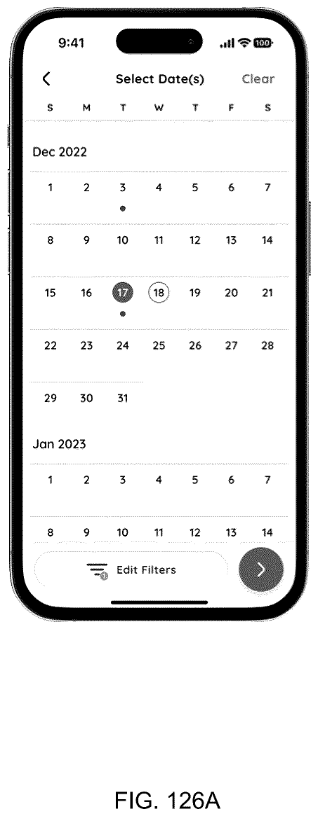

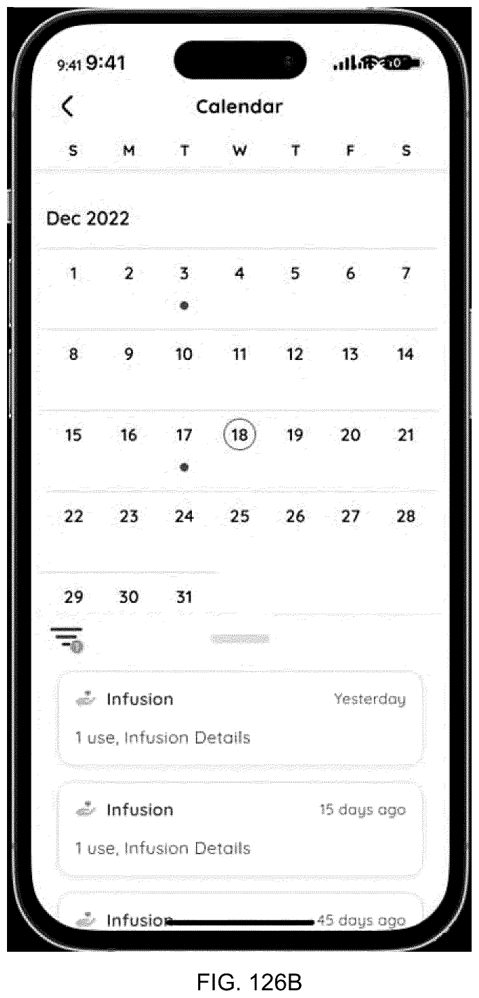

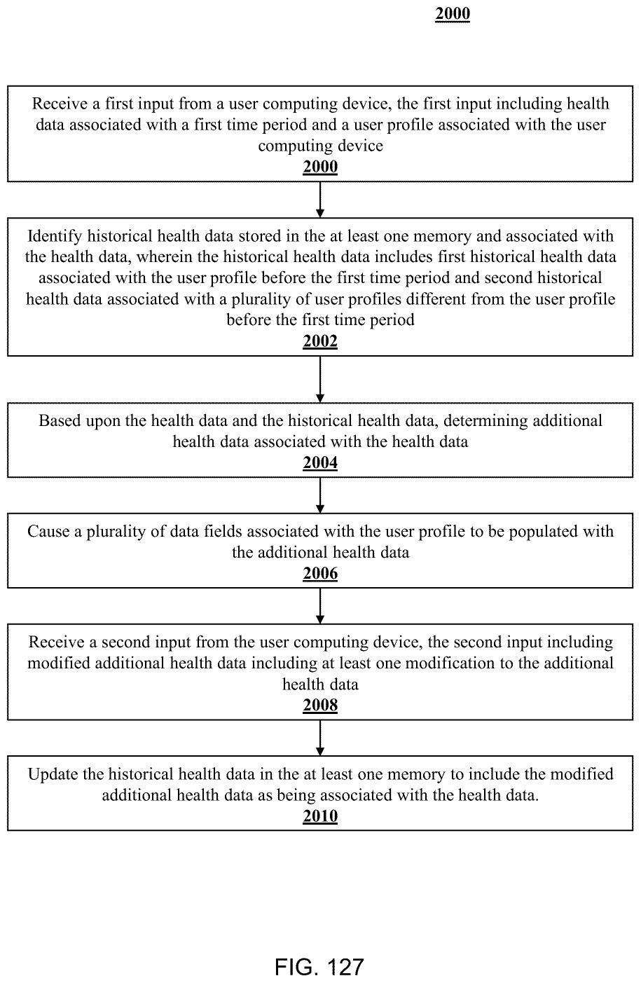

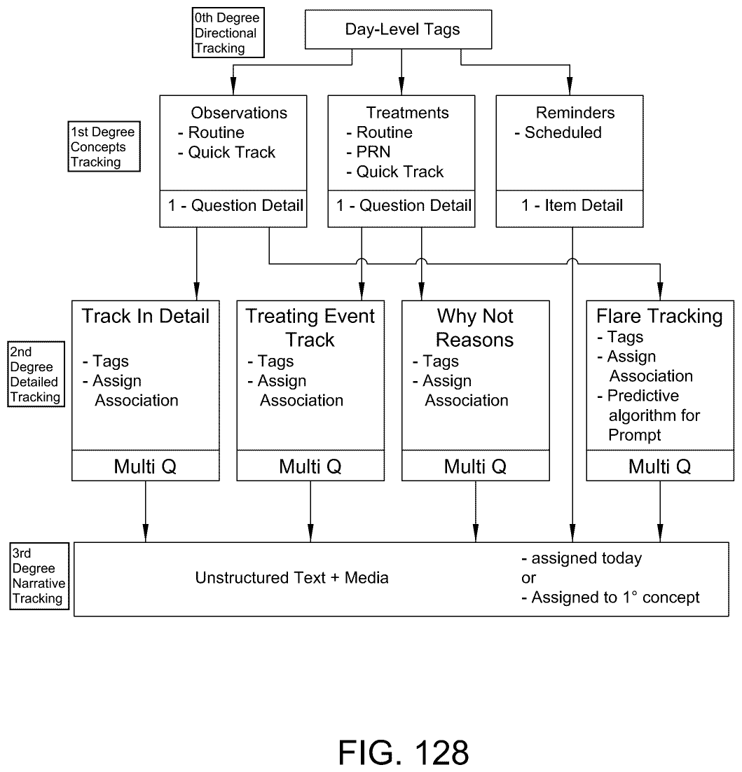

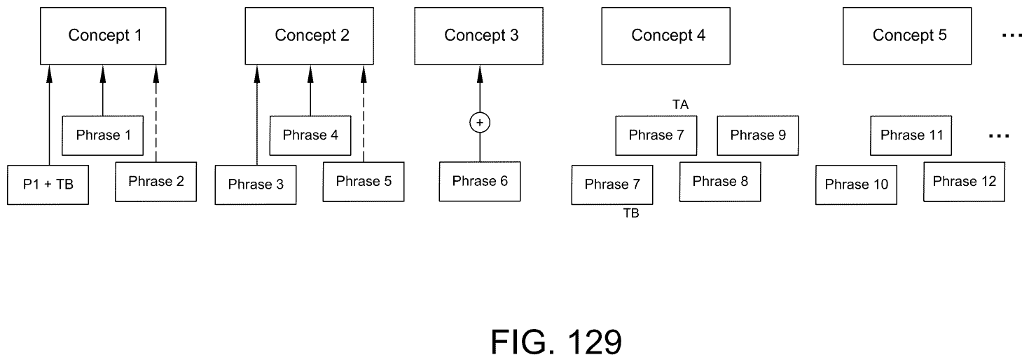

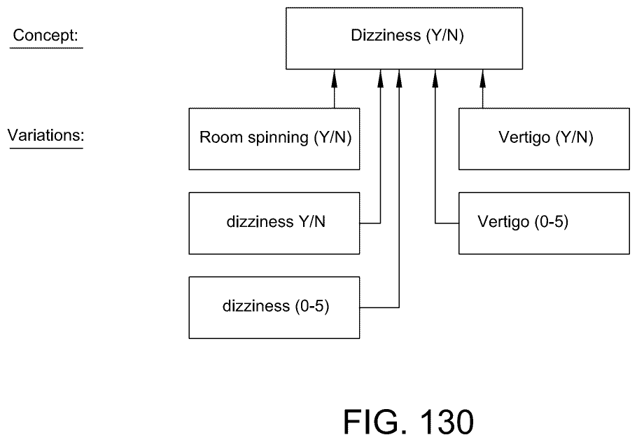

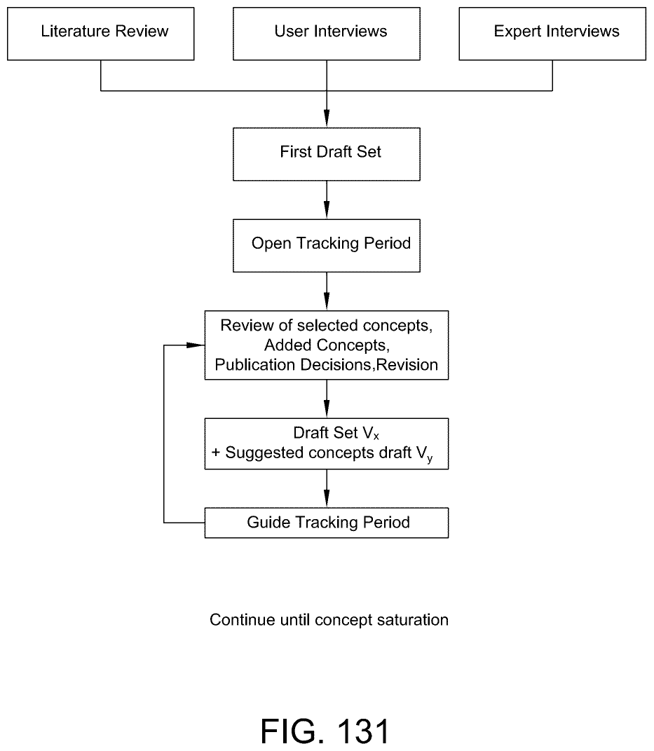

FIG. 25 illustrates a next GUI after the user selected track in FIG. 24. As shown in FIG. 25, a user may select certain keywords (e.g., from a dropdown and/or search bar) that describe how they feel that day. Further, a user may select an add selector to create their own entry (e.g., not from the drop down). FIG. 26 illustrates a GUI with example selectors provided in response to selection of the search keywords input area shown in FIG. 25. In the example of FIG. 26, calm sleep is selected. Accordingly, FIG. 27 shows a next example of the GUI shown in FIG. 25, including calm sleep as selected by the user in FIG. 26. FIG. 28 illustrates a next GUI providing the user the option to manually enter notes regarding their sleep, to supplement their selection of calm sleep, as shown.

Continuing the above example, FIG. 29 illustrates a GUI where the user is asked if they completed their Oxbryta treatment at breakfast, with selectors all done, some done, change amount, and not today provided. In this example, the user selects all done, prompting display of the GUI shown in FIG. 30 asking the user if they completed their dinner treatments (e.g., Folic Acid and Hydroxyurea). In response to selection of “Some Done” at the GUI of FIG. 30, the GUI of FIG. 31 is displayed asking the user to select which treatments were completed. As shown in FIG. 31, the user has indicated that the Folic Acid treatment was selected.

FIG. 32 illustrates a next GUI based on user selection of Folic Acid in FIG. 31 (e.g., and not Hydroxyurea). Accordingly, the GUI of FIG. 32 asks the user to log why they did not complete Hydroxyurea treatment, along with selectors to select a reason or skip selection. FIG. 33 illustrates a GUI displayed in response to user selection of select a reason in FIG. 32. The user is provided selectors associated with possible reasons, as well as the option to enter a custom reason. As shown in FIG. 33, the user has selected didn't have time.

Next, the user is asked if they had yellow eyes today at the GUI of FIG. 34, as prompted by the user selecting for yellow eyes to be tracked as yes/no (e.g., see FIG. 21). As shown in FIG. 34, the user can select yes or no.

FIG. 35 illustrates a GUI where a user can input a normal severity of fatigue in response to asking device 102 (e.g., via an input at their mobile device) to track their fatigue. Accordingly, FIG. 35 allows user selection, on a scale of 1-10, of their usual fatigue. As shown in FIG. 35, a user has dragged a selector from 5 (e.g., a starting position) to 7 (e.g., a final position) to indicate their normal fatigue is 7 out of 10. FIG. 36 illustrates a next GUI asking the user whether their fatigue is better, worse, or the same as usual, or if they would like to track fatigue in more detail. FIG. 37 illustrates a GUI following user selection of worse, and allowing the user to drag a selector to a different position between 1 and 10 (e.g., and including a baseline marker at 7). In the example of FIG. 37, the user has dragged the selector from 7 to 3.

FIG. 38 illustrates a GUI prompting a user to set baselines for depression (e.g., in response to the user prompting device 102 to track depression at FIG. 17). FIG. 39 illustrates an example GUI prompting the user to enter their usual depression on a scale of 1 to 10. As shown in FIG. 39, the user has selected 4. Device 102 then asks the user how their depression is today in the GUI shown at FIG. 40. In the example of FIG. 40, the user has selected same as usual (e.g., 4).

FIG. 41 illustrates a next GUI where a user is prompted to provide their daily water intake (e.g., in response to the selection shown in FIG. 18). Accordingly, a user can select to measure their water intake or select no measurement today. In response to selecting to measure their water, the GUI of FIG. 42 is provided and allows the user to input their daily water intake. As shown in FIG. 42, the user has inputted 58 oz as their water intake that day.

FIG. 43 illustrates a further GUI where the user is prompted to enter their hemoglobin (e.g., in response to the selection at FIG. 18), along with the options to measure their hemoglobin or skip measurement. FIG. 44 illustrates a GUI provided in response to user selection of skip at FIG. 43. In the example of FIG. 44, the user has completed their daily questions and is notified accordingly (e.g., and indicating a number of rewards earned (e.g., toward a next level) and the number of questions answered).

FIG. 45 illustrates an example GUI shown in response to user selection of an insights tab shown on the dashboard of FIG. 23, for example. As shown in FIG. 45, the user may select that a new graph be generated and displayed or may select quick insights. FIG. 46 illustrates a GUI shown in response to selection of the new graph selector shown in FIG. 45 and asks the user to select what they would like the graph to include. For example, in response to selection of the search bar shown in FIG. 46, the GUI of FIG. 47 may be provided to allow the user to select one or more options for display in the requested graph. FIG. 48 illustrates the GUI of FIG. 47 upon user entry of joint into the search bar, the results being filtered to options including the word joint, and user selection of joint pain (scale of 1-10).

FIG. 49 illustrates the GUI of FIG. 46, now including joint pain (scale of 1-10), as selected in FIG. 48. Notably, a user may select more than one option for a graph. As shown in FIG. 50, the user has indicated they would like an additional item be shown in the requested graph. After entry of “fatig” in the search bar, options including those letters are provided at the GUI shown in FIG. 51, and the user has selected “Fatigue (Scale of 1-10)”. As shown in FIG. 52, the user has requested yet another item be shown in the requested graph, has searched for “head”, and has selected “Headache (Scale of 1-10)”.

FIG. 53 illustrates the requested graph as generated by device 102 in response to the user selections shown in the preceding figures (e.g., showing data regarding joint pain, headache, and fatigue, on scales of 1-10, as informed by prior user daily entries).

FIGS. 54-59 illustrate example screenshots and/or GUIs (e.g., as shown at one of devices 108-112 based on data received from device 102 via API 104 and/or 106) for developing and utilizing participant-adjusted endpoints as implemented by PAEP system 100. Presented in these examples is a single platform combining personal health tracking with remote participation in research studies.

In one embodiment, data collected within the platform is available immediately for dual research and individual use. The user can track their personal health in one platform, and the data collected within that platform can be used for both managing their own personal health and sharing that data with external parties for research purposes. In one embodiment, PAEP system 100 includes a personal health tracking home screen including a research checklist area. The home screen of the PAEP system 100 may provide both guidance for personal health tracking and alerting the user when there is a separate task for them to complete as part of the Research Study they are enrolled in and have consented to be a part of. FIG. 54 illustrates an example home screen generated by the PAEP system 100.

In one embodiment, a research consent dashboard is included within the PAEP system 100. The user can manage their consent for both specific research studies that support clinical trials and general research within the same application where they track and manage their own personal health.

In one embodiment, the PAEP system 100 includes user-specific research sharing settings for user data. There may be a general research on/off setting, whereby the user has the opportunity to share their data within the platform to be used for general research purposes. By turning this general research setting on, they are consenting for their de-identified data to be aggregated and used for analyses.

In one embodiment, the PAEP system 100 includes a research history. Whenever the user's de-identified data is used for a retrospective analysis outside of a specific study the user has already specifically provided consent for, this analysis may be added to the user's analysis history, which they can view in their profile. This history serves as a record for the user to see how their data has been used in research.

In one embodiment, the PAEP system 100 includes research sharing for specific studies. Opting into a research study may prompt a consent flow. The user can manage with which specific studies they are sharing their tracking data. When the user consents to share their data, this action triggers a consent form that outlines the user's consent to share their data as part of the study.

In one embodiment, PAEP system 100 identifies one or more stages of a diagnostic odyssey and provides support for one or more of the stages. For people who do not have a diagnosed condition or do not think their diagnosis is accurate, PAEP system 100 allows them to identify which stage of the diagnostic odyssey they are in. There are various stages of the diagnostic odyssey, progressing from noticing symptoms to affirming diagnosis as defined by PAEP system 100. Each stage has a written description of what it is to help users identify the stage that most accurately reflects their current situation.

In one embodiment, PAEP system 100 tags days with summary tags to represent significant health developments and capture event-specific data. In one embodiment, PAEP system 100 includes flare tracking. Flares are a specific summary tag, where flare tracking refers to the user's ability to differentiate between days that contain “flare-ups” of their health versus days that are more typical. This differentiation allows a user to see spikes in their health over time, see what a typical flare looks like compared to a more normal day, and have data visualizations specifically around their flares. As part of this flare, the user can track a description of the flare, whether they did anything specific to manage the flare, any potential triggers of the flare, free-form written notes, among other details. There are differing versions of the flare tracking options to match different types of flares the user might experience. Based on the user's condition, PAEP system 100 will default to showing a specific flare type, which the user can manually change if they want different flare tracking options to more closely match the flare they are experiencing.

In one embodiment, PAEP system 100 may suggest that the user track a flare on one or more days. The user can choose to track the flare or dismiss the suggestion.

In one embodiment, PAEP system 100 provides a user with the opportunity to track other summary tags at the day-level, such as hospitalizations, emergency room (ER) visits, medical operations, treatment plan changes, new diagnoses, healthy/positive days, and custom summary tags of the user's own creation.

In one embodiment, PAEP system 100 includes responsive and customizable artwork for a daily health journal of a patient health tracking application. The user can choose the theme of the artwork that is shown in their daily health journal (e.g., forest and trees, ocean, lake, city life, etc.). The user may also have the option to upload their own image to put at the top of their journal.

In one embodiment, for one or more art scenes, the scene will respond to the time. For example, if it is between 7:30 PM and 5:00 AM, the scene will change to a night version, and if it is between 5:00 AM and 7:30 PM, the scene will be a day version. Some artwork scenes may also change based on the day of year, e.g., with some changing to a winter scene during winter, a spring scene during spring, a summer scene during summer, and a fall scene during fall.

In one embodiment, PAEP system 100 includes filtering of the calendar and daily journal to specific items. The user may have the ability to filter their journal to specific observations, treatments, or events. The journal will then only show items that match the applied filters. The user can see an overview of what information a day in their journal contains via indicators, which can show that they have tracked data for a day in the past or that they have an event coming up for a day in the future. When the user applies filters to the journal, the indicators showing what information will be shown for a day in the journal then also gets filtered. As an example, if the journal is filtered to just show the observation headache, then the indicator showing what days have data tracked for them will now represent days that have headache data tracked.

In one embodiment, flares and summary tags may be included in filtering. If a flare is tracked on a day in the past, the indicator for that day will be represented by a flare. Similar to the other indicators, this type of indicator may be filtered out if summary tags are not included in the applied filters. FIGS. 60-61 illustrate example screenshots and/or interfaces for flare tracking as implemented by the PAEP system shown in FIG. 1.

In one embodiment, PAEP system 100 includes a heat map. The heat map may be overlaid on a calendar or an expanded calendar and/or be responsive to filters. The heat map may be responsive to filters, such that the heat map will only display data that has been tracked with the selected filters.

In one embodiment, PAEP system 100 may represent scheduled treatments and events within a schedule (e.g., an hour-by-hour daily schedule). The user has the option to toggle the view of the day selected within the calendar page from the daily journal to the daily schedule. The daily schedule may be an hour-by-hour view of the information within that day, which defaults to showing just the user's treatments and events, including completion data for the treatments. The items displayed on the daily schedule may be responsive to filters, such that the heat map will only display data that has been tracked with the selected filters.

In one embodiment, PAEP system 100 includes suggested questions. Suggested questions provide dynamic user guidance by suggesting specific items to track on the basis of condition, age, or research status. FIGS. 55-56 illustrate example screenshots and/or interfaces for developing and utilizing participant-adjusted endpoints as implemented by the PAEP system shown in FIG. 1.

In one embodiment, PAEP system 100 includes suggested observations and treatments in onboarding. During onboarding, the types of observations and treatments that are suggested to the user to track in their journal will be customized based on the user's condition(s), age, and/or research status. The user may turn these suggested items on via pressing a toggle.

In one embodiment, PAEP system 100 includes a search tool to identify additional items beyond suggested questions. The user may interact with a GUI to perform a custom search for other observations and treatments that are not shown in the suggested grouping.

In one embodiment, PAEP system 100 includes smart suggestions. Smart suggestions may be algorithmic suggestions based on how people are using the platform. These suggestions may be based on the user's condition(s), age, and/or research status and may respond to user usage. The most commonly turned on items may be ranked higher up on a list of suggestions, and items that meet a usage threshold may be added to the list of suggestions.

In one embodiment, PAEP system 100 includes user-triggered suggestion refreshing that incorporates recent tracking. Outside of onboarding, the user may also re-trigger the suggested list of observations and treatments, and this list will be responsive to the user's tracking history in addition to the user's age, condition(s), and/or research status.

In one embodiment, PAEP system 100 includes platform-triggered suggestions based on tracking history. Outside of onboarding, PAEP system 100 may also suggest specific observations and treatments to track based on the user's tracking history.

In one embodiment, PAEP system 100 allows for individually flexible observation tracking within journal data capture. When tracking specific observations, users may have the flexibility to change the observation type so they can track observations in whichever data capture method most closely suits their tracking and visualization needs.

In one embodiment, PAEP system 100 may track additional detail via consistent, discrete tags. Whenever a user tracks an observation, they may have the ability to add tags. Tags are details of an observation that respond to a specific question or area of that observation and can be recorded consistently across observation sessions. As an example, if the user is tracking coughing, in addition to tracking the severity of the coughing, they could also track details such as the type of cough (e.g., dry, contains blood, etc.) via tags. Because tags are consistent, discrete values within an observation, they can then be visualized on a graph in addition to the core tracking value (like severity).

In one embodiment, PAEP system 100 includes user-customized tags. Tags can also be customized, such that the user can create their own tags within a grouping, and remove pre-existing tags that they do not wish to use.

In one embodiment, PAEP system 100 includes universal tags for observations. Universal Tags are a tag grouping that is present and consistent across every observation, so that if the user wants to track a specific tag across different observations, they can do so easily without having to re-create the tag in each observation.

In one embodiment, PAEP system 100 includes one or more time-based treatment groups and/or one-click scheduled treatment completion. Treatment tracking refers to the ability for the user to track completion data on which treatments they have taken, or which treatments from their treatment plan they have skipped. As shown in FIGS. 57-58, the user may have the ability to mark all scheduled treatments for a day complete in one click of a mark all complete button.

In one embodiment, PAEP system 100 includes time-based treatment groups. The user can create time-based groups of treatments, which display as grouped within the journal.

In one embodiment, PAEP system 100 allows users to provide reasons for skipping a treatment. When a treatment is skipped, the user has the ability to track a reason for skipping that treatment.

In one embodiment, PAEP system 100 includes pre-visit preparation and/or post-visit outcome tracking for visits and events. Visits and events tracking refers to the user's ability to track significant events like doctor's appointments within PAEP system 100. In pre-visit preparation, PAEP system 100 provides the user with a specific pre-visit flow to prepare what data they would like to show the doctor and/or what questions they have for the doctor.

In one embodiment, PAEP system 100 allows specific visualizations to be added to an event (e.g., a doctor's appointment). The user may create specific visualizations within an insights page, and they may add those visualizations to upcoming visits and events in the calendar. The user may also add platform-generated reports to their upcoming visits and events.

In one embodiment, PAEP system 100 includes open questions for doctors. The user may create a list of questions they have for their doctor and then choose to include those questions within their upcoming visits and events.

In one embodiment, PAEP system 100 includes post-visit outcome tracking. The user may keep track of any changes to their treatment plan/schedule that were made or suggested from specific visits and events. The user may keep track of which questions were resolved by the doctor from specific visits and events, and answers to questions from specific visits and events.

In one embodiment, PAEP system 100 includes heat map standardization in visualizations across observation types. When the user adds multiple observations into in a visualization, the visualization may be manually or automatically converted into a heat map so that the user can see peaks and valleys of the observations across different observation types. FIG. 59 illustrates an example screenshot and/or interface for heat map visualization as implemented by PAEP system 100.

In one embodiment, PAEP system 100 includes a readable simple report containing a consumable written overview of the user's health, dynamic to recent tracking patterns, which is supplemented by visualizations. The readable simple report may be a readable and easily consumable written report of recent tracking history. The report may be dynamic to recent tracking patterns. The written report may have the option to be supplemented with graphs to put a visual to the written report.

In one embodiment, PAEP system 100 includes condition-specific platform customization. Some areas of the platform are condition-responsive and may be customized to more closely match the experience of particular conditions.

In one embodiment, PAEP system 100 includes condition-specific suggested questions. The types of observations and treatments that are suggested to the user to track in their journal will be customized based on the condition the user selected.

In one embodiment, PAEP system 100 includes condition-specific suggested treatment schedules. The default schedule suggested for specific treatment may be customized based on the condition the user selected.

In one embodiment, PAEP system 100 includes condition-specific suggested visualizations. The recommended visualizations may be customized based on the condition(s) the user selected.

In one embodiment, PAEP system 100 includes a unique caregiver and/or patient account structure. On account creation, the user may choose between being a caregiver or a patient. The user may add a caregiver profile or additional patient profiles. The user may switch between the caregiver profile and any patient profiles they have within their account to track for multiple profiles.

### Home-Reported Outcomes

PAEP system 100 may store journal data, which includes all health observations made in a journal (e.g., an electronic journal that is stored with respect to a profile). Journal data may include observation severity, count, presence (e.g., yes/no), numeric values with a unit, tags, timings, duration, free text note, voice notes, and/or media images. Journal data may include treatment completion status, amount, tags, timing, and/or free text notes related to treatment completion status. Journal data may include medical visit information (including location and who was involved), free text notes, and/or voice notes related to medical visits. Journal data may include flare presence (e.g., yes/no), tags, free text notes, voice notes, and/or media images related to flare presence. Journal data may include general data on a day (e.g., was it a difficult, typical, or good day, other day-level tags for context around relevant daily details like sleep, meals, social connections, or more, as well as day-level notes and day-level media images). The functions and methods described below may be implemented on a user interface, for example, on user device 110, to accept user input and display information to a user (e.g., as caused by PAEP system 100 and/or one or more machine learning models associated therewith).

PAEP system 100 may include a platform and method for collecting, structuring, and/or analyzing journal data so that proper analysis can be conducted across individuals and time, including establishing a taxonomy of the journal data and aggregation and translation of journal data and other data sources into a data structure for consistent analysis. The taxonomy may include at least one or more journal items, pieces of journal data, journal item tags, and/or journal item filters, described below. The taxonomy standardizes symptom tracking and symptom definitions, providing a universal set of tags, items, definitions, and/or filters that are available to all users.

PAEP system 100 may include one or more journals, the one or more journals including one or more journal items. A journal of the one or more journals may be associated with a user. PAEP system 100 may further include a comprehensive set of one or more journal items. Each journal item may be individually flexible, such that an item type and data structure of the journal item are alterable or customizable by the user. Users may create items not already present in a default set of journal items stored in knowledge base 706.

Each journal item may include one or more pieces of journal data. Journal items may have one or more mutable properties—for example, the number of pieces of journal data and the type of journal data associated with the journal item. The one or more mutable properties may be updated, changed, and/or altered based on user input. The one or more mutable properties may further be updated, changed, and/or altered a determination or calculation by PAEP system 100 that the mutable properties require an update—for example, if a majority of users are adding a “duration” journal data field to the “headache” symptom, PAEP system 100 may automatically update the default “headache” symptom to add a “duration” journal data field to the default journal item associated with headaches.

PAEP system 100 may include a crowd-sourced database of journal items, including observations, treatments, tags, flare tags, and/or summary tags that are editable and re-published over time to ensure that journal data capture default values and that journal data capture suggestions stay relevant as more information is gained about how individuals use the journal. The PAEP system 100 may further include a function and method for developing a common language or structure for symptom tracking & definitions.

PAEP system 100 may include a suggestion function and method to suggest journal data capture items (including observations, treatments, flare tags) to individuals based on aspects of the individual's previously reported journal items or health information, including: one or more of their reported diagnosed or suspected conditions; the health observations and treatments that individuals have previously recorded; the research studies they are currently or have previously been participating in; areas of health improvement or health goals that they have identified as priority areas; age; gender; and/or other demographic information. PAEP system 100 may determine, based on journal items previously entered by the user, one or more predicted journal capture items, then suggest the journal data capture item to the user to prompt the user to enter data associated with the suggested journal item.

PAEP system 100 may include a periodic review function and method to deliver a periodic review of journal data to confirm its accuracy and to request a user to add additional information or context as necessary to specific accounts based on aspects of an account's previously reported health information, including: one or more of their reported diagnosed or suspected conditions; one or more health observations and treatments that the account has previously recorded; one or more research studies the account is currently or has previously been participating in; areas of health improvement or health goals that the account has identified as priority areas; age; gender; and/or other demographic information.

PAEP system 100 may aggregate a summary of one or more journal items associated with a timeframe. The timeframe may be determined by a user or may be a default timeframe as determined by PAEP system 100. The summary may be aggregated based on journal items contained within the timeframe. PAEP system 100 may determine one or more missing journal items, data fields, and/or other missing information based on the summary. If missing items are determined, PAEP system 100 may prompt a user to enter one or more missing items (e.g., data fields).

PAEP system 100 may include a baseline function and method for setting a baseline for individual journal items for severity-based observations, being able to track when a value is the same, better, worse, and/or different in relation to the baseline. For other observations, PAEP system 100 may include a function and method to track a value with the baseline serving as a preset value for a given day. FIGS. 62-67 illustrate example embodiments of the baseline function and method implemented on a user interface, for example, on user device 110. FIGS. 62-64 illustrate a symptom with no baseline, a symptom tracked with a non-severity baseline, and a symptom with a severity baseline, respectively, for specific symptoms. FIGS. 65-66 illustrate tracking a symptom against a severity baseline, where the symptom “brain fog” has the baseline severity set to moderate. FIG. 67 illustrates the baseline function and method tracking a symptom by count rather than severity.

PAEP system 100 may determine a baseline associated with a journal item based on user input and/or or may determine or calculate a baseline value based on other factors, such as average values from all journal items associated with the “headache” tag. For example, a user may select that their baseline severity for “headaches” is a value of three. The selected baseline value will be the default selection for future entries of the “headache” journal item for the user, and future calculations or determinations made by PAEP system 100 for the user may use the baseline value for headache as a point of comparison.

In one embodiment, knowledge base 706 of PAEP system 100 may include a central database of default journal items available to track. Each journal item of knowledge base 706 may be individually flexible, such that differing levels of detail or pieces of journal data may be associated with the journal item and stored. Users may manually create custom journal items and/or PAEP system 100 may automatically generate custom journal items. PAEP system 100 may publish new default journal items based on the crowd-sourced, custom journal items created by users. Publication of new journal items may occur based on user input, administrator input, or based on a calculation or determination by PAEP system 100 that a threshold value has been reached. For example, PAEP system 100 may automatically publish a new journal item if a threshold amount of users have created a custom variation of the same or a similar journal item.

PAEP system 100 may include a day tracking function and method and method to track whether a day or range of days was difficult, typical, good, or great via both the home screen and the journal. FIGS. 68-76 illustrate example embodiments of the tracking function. PAEP system 100 may cause display of an overview of the day's tracked information, both before and after the user goes through the tracking flow. The overview may include core sections of the journal (e.g., observations, treatments, what else happened) and within those sections, may include items with similar values on similar lines with a specific icon in the overview card (e.g., grouping observations that were worse than usual, grouping As Needed/PRN treatments). PAEP system 100 may suggest specific groups of data capture based on the type of day tracked (As Needed/PRN treatments or flares on a difficult day). PAEP system 100 may prompt a user to enter one or more journal items associated with one or more days. FIGS. 68-70 illustrate examples of different data entry prompts for tracking days. FIG. 68 illustrates a prompt for a user to track the current day. FIG. 69 illustrates a prompt for a user to enter tracking data for a previous data. FIG. 70 illustrates a prompt for a user to enter tracking data that defaults to a typical data, or is similar to another day that has already been tracked. FIGS. 71-72 illustrate an example embodiment an overview card for difficult and typical days, respectively.

PAEP system 100 may suggest individual day tags associated with a day based on the type of day tracked. PAEP system 100 may suggest context tags to track, where context tags were used previously on that specific type of day. FIGS. 73-74 illustrate example embodiments of suggested individual day tags for difficult days and great days, respectively. Suggested tags may vary based on the type of day being tracked. Context tags may be colored coded and grouped based on one or more criteria.

PAEP system 100 may prompt the user to go through a guided flow for each type of day that is catered to that type of day (e.g., a typical day flow will streamline that things are the same as usual and that all treatments were completed, while a difficult day flow will ask what was worse than usual, and if the user used an as needed/PRN treatment or had a flare). For example, the guided flow may be catered to weekends, weekdays, or other types of days as indicated by a user using either default or custom day types from the knowledge base. The guided flow defaults information in the user interface and selection menus to a baseline value based on the type of day being tracked. FIGS. 75-76 illustrate an example embodiment of a guided flow. FIG. 75 illustrates a first prompt for the user to enter baseline information on symptoms or other items. FIG. 76 illustrates a prompt to enter any treatment information or other items.

PAEP system 100 may calculate and display one or more daily trends to the user. PAEP system 100 may display an overview of one or more journal items over a period of time. After tracking a day, PAEP system 100 may display daily trends to the user based on a selected day's tracked information relative to the past several weeks (for example, “this was your 8th Great Day this month”, “this was your 2nd Great Day in a row that included seeing family”).

PAEP system 100 may include a one-click tracking function and method for one-click treatment group tracking of the completion value for a specific day. PAEP system 100 further includes a function to group treatments that are taken together into one group, where all treatments in the group can be completed or skipped together in the same click. FIG. 77 illustrates an example embodiment of a treatment group. Each item of the treatment group may be selected by default, and the user may select or deselect items to customize the tracking.

PAEP system 100 may include a one-click all treatment tracking of the completion value for a specific day. The user may complete all treatments in a day in one-click. FIG. 78 illustrates an example embodiment of the one-click all treatment tracking being selected to track completion of all treatments. FIG. 79 illustrates an example prompt offering one-click completion when a user is prompted with any skipped scheduled treatments.

PAEP system 100 may include a voice prompt function and method. The voice prompt may capture home-reported outcomes data via input audio from the user. For example, a user may say “today, all my baseline symptoms were worse than usual, and I completed all my treatments, and took 2 pills of Ibuprofen”. PAEP system 100 may convert this audio to text and store the text. Text generated by a voice prompt may be processed and segmented, and one or more journal items may be generated or edited based on the generated text. In the above example, a journal item for the day is generated showing the baseline systems are worse, all treatments were completed, and adding 2 pills of Ibuprofen to a medication section of the journal item.

PAEP system 100 may provide streamlined tracking of baseline symptoms and scheduled treatments based on a typical day. PAEP system 100 may determine, based on a day type, one or more suggested journal items and display the one or more suggested journal items to the user. For example, if a user indicates that a day is “typical”, PAEP system 100 may suggest and prepopulate journal items and baseline values for journal items previously appearing in “typical” days entered by the user. FIGS. 80-81 illustrate an example embodiment of a streamlined tracking of baseline symptoms prompt. FIG. 80 illustrates a prompt to the user providing pre-filled baseline values for each symptom. FIG. 81 illustrates a prompt for a user to enter any skipped treatments for the day, using the typical day as a baseline.

PAEP system 100 may further include a copying function and method to copy the observation and as needed/PRN values from a previous day and apply them to a different day in the journal. Other properties associated with a journal item representing a previous day may also be copied and applied to a different day. FIGS. 82-84 illustrate an example embodiment of the copy function to copy values from previous similar days. FIG. 82 illustrates an example prompt to track the day as similar to another day. FIG. 83 illustrates an example prompt suggesting values from previous similar days. FIG. 84 illustrates an example overview after a user selects a previous similar day, showing a summary of the journal items and tags associated with the selected previous day.

PAEP system 100 may include a previously tracked day saving function and method for saving the observation and as needed/PRN data from a previously tracked type day and naming or grouping the previously tracked observation and as needed/PRN data to track that set of data again in the future. Previously tracked days may be pinned, saved, or otherwise held for future use. FIG. 85 illustrates an example previously tracked day saving function for pinning a tracked day to a portion of the user interface for future re-use.

PAEP system 100 may include a suggestion function and method to detect if a user has not tracked journal items for a few days and suggest or prompt the user to track a group of days together based on the detecting. FIGS. 86-87 illustrate an example embodiment of the suggestion function, noting that the user had not tracked data for the last 7 days, and prompting the user to track data for the missing day or days.

PAEP system 100 may include a detailed capture function and method for capturing structured, additional detail to a specific journal data point. PAEP system 100 may include a default tag function for default tag groups and tags to be set for specific journal items via the knowledge base. PAEP system 100 may include a custom tag function allowing the user to add custom tags to specific journal items.

PAEP system 100 may include a function and method allowing the user to track journal tags to specific journal data points for additional data capture. Journal items or elements of journal items may be associated with a journal tag. For example, a user may tag an item with default or custom tags, including but not limited to tags related to severity, importance, differences with previous days, or other information. Tags may be used to capture information regarding additional characteristics of an observation, such as the description of a sensation, the location of the associated journal item, or anything else related to the overall health observation that is supplementary to a core tracking value.

PAEP system 100 may include a tag skip function and method to leverage the tag system to capture reasons for skipping a treatment on a given day. Upon a user marking a treatment as skipped, the tag skip function may prompt a user to enter one or more reasons why the user skipped treatment for a day or may prompt a user to enter a reason for skipping a treatment when the treatment is initially marked as skipped. FIG. 88 illustrates an example tag skip function, prompting a user to enter one or more pre-determined reasons why the user skipped treatment for a day. The user may also be prompted to enter the reason in an open-ended text prompt. FIG. 89 illustrates an example tag skip function, prompting a user to add reasons for skipping a treatment when the user marks the treatment as skipped.

PAEP system 100 may include a media tracking function and method for the user to track media files. The PAEP system 100 may analyze media files and provide a specific value for data capture of a corresponding journal item.

PAEP system 100 may include a structured tag function and method for structured tag data capture for the user to track specific location tags. PAEP system 100 may include an interactive body map for the user to interact with and track specific locations on a digital body representation as tags. PAEP system 100 may create journal items based on the user's selections on the interactive body map.

### Flare Tracking

PAEP system 100 may include a flare tracking function and method to tag a day (or set of days) with the presence of a flare. Flare tags may be associated with one or more journal items. Flare tracking function may further include capturing additional information about a flare, for example, by user input. FIGS. 90-102 illustrate an example embodiment of the flare tracking function. FIG. 90 illustrates an example embodiment of a prompt for a user to add a flare. Flare tracking function may prompt a user to enter information about what steps the user is taking to manage the flare. FIG. 91 illustrates an example of a symptom tracking interface following the addition of a flare. FIG. 92 illustrates an example prompt for a user to enter flare management tags. PAEP system 100 may prompt a user to enter flare triggers or causes, as shown in FIG. 93. PAEP system 100 may prompt a user to enter one or more flare symptom tags, as shown in FIG. 94. PAEP system 100 may prompt a user to enter one or more custom flare symptom tags, which may be saved and store for future flares, as shown in FIGS. 95, 96A, and 96B.

PAEP system 100 may include a flare suggestion function and method that includes creating a suggestion and/or prompt for a user to track a flare based on one or more parameters. For example, the one or more parameters may include how the user is tracking a specific day or set of days and/or the user's personal health information or existing journal items. The existing personal health information or existing journal items may include what condition and customized flare form the user has, such that the suggestion and/or prompt is based off the condition and customized flare form. FIG. 96A shows a prompt or suggestion for a user to track a flare based on a detection that the user's symptoms are more severe than usual.

PAEP system 100 may improve and/or update the flare suggestion function for a particular condition based on data or journal items from a plurality of users (e.g., historical data). For example, the flare suggestion function may be improved and/or updated based on the accuracy of flare suggestions to the plurality of users (for example, how often users are tracking flares when a flare suggestion is triggered) and retroactive analysis on most commonly correlated observations to flare tracks from the plurality of users. One or more machine learning models may be initially trained and/or trained over time based upon, for instance, the accuracy of flare suggestions and/or other data described herein.

PAEP system 100 may include a flare tag function and method to track structured details to a specific flare in the form of flare tags. Flare tags may be associated with a tracked flare. Flare tags may include description of the flare, what the user is doing to manage the flare, and any possible triggers for the flare. The user may add custom flare tags. Custom flare tags may be stored, and the user may be prompted to apply custom flare tags to future flare entries. FIGS. 95, 96A, and 96B illustrate an example prompt for a user to track predetermined flare tags, prompt a user to mark a date as a flare based on user-entered journal items or data, or enter a custom tag for a flare.

PAEP system 100 may customize the flare content and structure. Specifically, PAEP system 100 may determine what flare tag groups appear in a flare as well as the default flare tags that appear based on the user's personal health information. Default flare tags shown to a user may be based on data aggregated or calculated from knowledge base 706.

PAEP system 100 may prompt a user to enter and track free-text notes and media to a corresponding flare, as shown in FIG. 97. Entered data may be associated with the corresponding flare or a corresponding journal item.

PAEP system 100 may include a flare visualization function and method to visualize the presence of flares on all versions of the day-by-day calendar, as shown in FIG. 98 and FIG. 99. Flare visualization may include marking, highlighting, grouping, color-coding, and/or otherwise indicating the presence of a flare for one or more days.

PAEP system 100 may prompt the user to determine if the flare is still ongoing and if the user tracked a flare the previous day. PAEP system 100 may associate a flare with a day based on the day's journal items, for example, symptoms, treatments, context tags, and more. FIG. 100 illustrates a follow-up prompt on a day following a flare, prompting a user to enter if a flare is still ongoing. FIG. 101 illustrates a prompt for a user to enter information on the flaring symptoms, associating the symptoms with the day's journal items. FIG. 102 illustrates an example prompt for a user to tag pain type and pain location associated with a flare.

PAEP system 100 may include a flare association function and method to associate a flare into other journal items of a selected day (for example, symptoms that are worse than usual, As Needed/PRN treatments, and more). The flare association function associates the flare with one or more other journal items of the selected day.

### Participant-Adjusted Endpoints

PAEP system 100 may include a framework to accurately measure outcomes experienced in the course of living with a disease, and to establish a more robust understanding of the impact of new therapies, which crucially accounts for individual variation.

PAEP system 100 may include a function and method for capturing the unique experience of each individual to account for important signals regarding the efficacy of a new therapy and the current state of an individual's health.

To better address individual variation in disease manifestation and to more comprehensively describe the impact of therapy, PAEP system 100 may include participant-adjusted endpoints (PAEPs). PAEP system 100 may include a framework that accounts for individual variation by enabling measurement of different outcomes-of-interest among study participants, with population analysis focused on comparison of change at the domain level, and/or overall degree of change in disease burden experienced by participants.

### Two Birds/One Scone Research Concept

PAEP system 100 may include a platform for both capturing home-reported outcomes for the purpose of health management and contributing those outcomes to advance health research.

PAEP system 100 may include a journal for health data capture, systems and methods for integrating third-party data, and/or health management tools for visualizing and providing insights on the journal data. Tools for visualizing and providing insights may include graphs, charts, virtual body maps, overviews, summaries, synopsis, and/or other visualization or summarization tools. Visualization and insights may be created by PAEP system 100 based on user input, or may be automatically generated based on one or more criteria.

PAEP system 100 may include a function and method for users to consent to studies and contribute user data to advance health research.

PAEP system 100 may include a function and method for serving specific research questions and prompts to individual users based on their personal health information to increase the relevancy of research questions (e.g., asking the user about their perception of the effectiveness or impact of a specific medication that they have taken in the time period of the research). PAEP system 100 may determine one or more research questions and prompts that are relevant to a user based on the user's associated journal items and other health information. PAEP system 100 may transmit the one or more research questions and prompts to the user.

PAEP system 100 may include a function and method for a user to join and consent to a research study, participate in the research study by completing specific research prompts, manage the user's status in the study, receive payments associated with the study, and/or access resources (FAQ, health tips, etc.) associated with the study.

PAEP system 100 may prompt a user via a home screen to answer new prompts via a research homepage. The research homepage may further include a research checklist. The research checklist may display all potential, relevant, and/or active research opportunities to the user. FIG. 103 illustrates an example prompt for a user to view the research checklist from a dashboard page. FIG. 104 illustrates an example research homepage showing the research checklist. FIG. 105 illustrates an example expanded research home page showing multiple selectable options, include checking statuses of research, viewing an FAQ, viewing quick tips, and viewing one or more other resources.

A user can view, using a user interface of PAEP system 100, when the user has new payments or rewards available to the user. The research homepage is accessible via the home screen to the user. FIG. 106 illustrates an example prompt for a user to redeem research rewards. FIG. 107 illustrates an example research prompt viewable from a journal entry page.

PAEP system 100 may further include a revenue function and method for sharing the revenue from research studies to the participants of the research studies via rewards, such as gift cards, payments of legal tender, virtual credits, and/or other incentives.

### Chapters

PAEP system 100 may include a chapter function and method for creating a thematic, annotated timeline of health events, trends, and progressions.

The chapter function may create a chapter or sub-group of trends, notes, graphs, events, media over a period of time, resulting in a “zoomed out” view of the journal with annotated health trends. Chapters may be created based on user selection or automatically generated by PAEP system 100 based on one or more journal items.

PAEP system 100 may prompt a user with chapter suggestions to guide the user to create a chapter. Chapter suggestions may be determined and triggered by PAEP system 100 detecting one or more criteria from the user's journal data, such as initiation of a new treatment. FIG. 108 illustrates an example of a new chapter suggestion based on detecting a first week of new treatment.

PAEP system 100 may include a function to filter one or more chapters based on specific information in the chapters, a function to quickly search chapters for a particular phrase, a function to group chapters for horizontal scrolling, and an AI-generated summary of chapters based on one or more journal items contained in one or more chapters. The AI-generated summary may be generated by, for example, an AI component (e.g., computer module). The AI component may include, for example, a machine learning model such as a neural network. The AI component may be trained using a training data set including existing chapters created by a plurality of users (e.g., historical data). Inputs for the AI-generated summary may include a set of journal trends which typically precede the creation of a chapter both broadly (e.g., among all of the plurality of users) and for users of the plurality of users with similar personal health information (e.g., started a new treatment, new symptoms tracked, symptom severity getting significantly worse or better), and based on the user's current health trends. The AI component may output a chapter suggestion which prompts a user to create a chapter based on the user's journal trends. The AI component may be trained over time based upon which chapter suggestions are kept as suggested by the AI component and which chapter suggestions are modified (e.g., by a user).

FIG. 109 illustrates an example timeline overview of chapters, displaying summaries of different chapters. FIG. 110 illustrates an example of chapters grouped by journal items having AI-generated summaries, for example, grouped by disease, treatment, or other criteria. FIG. 111 illustrates an example prompt for chapter creation. FIG. 112 illustrates an example overview of a prompt to include journal items into a created chapter. FIG. 113 illustrates an example for confirming addition of journal items to a new chapter.

PAEP system 100 may cause display of chapters on a calendar, including all days the chapters span. The user may switch views from a timeline view to a view of chapters grouped by journal item.

### Advanced Date Selection for Journal Trends, Visualizations, Chapters

PAEP system 100 may include an insights function and method for the user to receive insights (e.g., via journal trends, visualizations, and/or chapters) over time periods that are specific to journal data, and not just generic time periods. For example, a user may receive insights related to a specific symptom or a specific tracked flare.

PAEP system 100 may include a specific item function and method for selecting a date included in a date range on the basis of a first or last data point of a specific journal item. FIG. 114 illustrates an example prompt for selecting a date range based on either a first track or a most recent track of a specific journal item. FIG. 115 illustrates a prompt for selecting a first track of specific journal items. FIG. 116 illustrates a prompt for confirming a selection of a specific journal item to track within a date range.

PAEP system 100 may include a date selection function and method for selecting a date included in a date range on the basis of a specific value tracked for a specific journal item. The selecting may include an option to center the date selection around the first of a specific tag or value of a journal item, (e.g., the first track where the user tracked joint pain in the left ankle).

PAEP system 100 may include a keyword function and method for selecting a date included in a date range based on specific keywords or phrases in journal data, like a note (e.g., the first date where the user wrote a note including “marathon”). Users may enter a keyword and a date range, and PAEP system 100 may cause display of one or more dates in the date range that contain a matching keyword to the user's input in an associated journal item.

### Journal and Search Filters

PAEP system 100 may include a filtering function and method for a user to filter, search, group the user's journal data. FIGS. 117-126B illustrate an example embodiment of the filtering function implemented on a user interface, for example, on user device 110.

The filtering function and method may include displaying a high-level overview of journal day(s), grouping similar journal data like as needed/PRN treatments into an overview card which clearly labels if as needed/PRN treatments have been tracked on that day, with drill-down filters in the journal available to see just the journal items in the grouping. FIGS. 117-118 illustrate an example of drill-down filters, the drill-down filters including the journal items available within a filtered grouping for each day. Drill-down filters may further filter or limit the display items associated with the filtered journal items.

The filtering function and method may further include a keyword search function for searching for specific keywords or phrases, generally or within specific journal items, such as notes (e.g., show the user when the user used the phrase “infusion” in any note in the journal). The filtering function may further include filters for specific values of specific journal items, either of the primary tracking value or the additional detail (e.g., filter to days when the user's coughing has been tracked as severe). FIGS. 119-122 illustrate example filter and sub-filter selections for filtering by days, observations, treatments, flares, tags, and/or notes. The filtering function may include combinations of search, filters, and/or drill-down filters. Days containing journal items or other data that match an applied filter may be highlighted, color-coded, selected, or otherwise indicated to the user. FIGS. 123-126B illustrate example implementations and calendar views of one or more applied filters, highlighting or otherwise indicating calendar dates that match the applied filters.

PAEP system 100 may display which days match the filters as denoted by a dot or other marker, and permit a user to scroll through the various tracked data points in the Journal that match the filters as shown in FIGS. 126A and 126B, for example, using a bottom half-modal display. When in this view and selecting a day that matches the filters, the bottom half-modal will scroll to display one or more tracked data points that are associated with the selected day.

FIG. 127 illustrates an example method 2000 for healthcare management. In some embodiments, a method for healthcare management is implemented by at least one processor in communication with at least one memory, the method including: receiving 2002 a first input from a user computing device, the first input including health data associated with a first time period and a user profile associated with the user computing device; identifying 2004 historical health data stored in the at least one memory and associated with the health data, wherein the historical health data includes first historical health data associated with the user profile before the first time period and second historical health data associated with a plurality of user profiles different from the user profile before the first time period; based upon the health data and the historical health data, determining 2006 additional health data associated with the health data; causing 2008 a plurality of data fields associated with the user profile to be populated with the additional health data; receiving 2010 a second input from the user computing device, the second input including modified additional health data including at least one modification to the additional health data; and updating 2012 the historical health data in the at least one memory to include the modified additional health data as being associated with the health data.

In some embodiments, method 2000 includes receiving a third input from the user computing device, the third input including second health data associated with a second time period and the user profile, wherein the second health data includes at least one data entry that is the same as the health data; identifying the updated historical health data in the at least one memory; and causing a second plurality of data fields associated with the user profile to be populated with the modified additional health data.

In some embodiments, method 2000 includes generating a calendar associated with the user profile; and causing an icon to be added to a portion of the calendar associated with the first time period, wherein the icon is associated with the health data.

In some embodiments, method 2000 includes, based upon the historical health data and the modified additional health data, determining one or more suggestions for a user associated with the user profile to address a health condition identified in the modified additional health data.

### Adoption of Common Question Language in Data Used for Analysis

PAEP system 100 may further include a standardization function and method for standardizing language through the taxonomy of data used to collect health research data. Standardization may be based on one or more criteria, including at least official medical publications, user-generated content (such as journal items), user suggestions, administrator decisions, or some combination thereof.

The taxonomy of data may include a multi-level data structure, as shown in FIG. 128. For example, the taxonomy may include a top level of directional tracking, a first level of concept tracking, a second level of detail tracking, and a third level of narrative tracking. PAEP system 100 may include dependency logic, such that PAEP system 100 may suggest a user include a next level of tracking upon determination that a threshold value has been reached by user's actions. For example, PAEP system 100 may suggest or prompt a user to track a value in greater detail if the value appears to deviate from existing data in some way. The threshold value may be set as a determination in response to a learning algorithm of the AI component, or may be set manually by an administrator, for example, in response to an analysis of existing data from a plurality of users or other research data.

In some embodiments, the first level of concept tracking may include observations, treatments, and reminders. The first level of concept tracking may involve a single question prompt to the user, or one or more question prompts. The second level of detail tracking may include more detailed or more numerous questions determined in response to user entries for the first level of concept tracking. The second level of detail tracking may include one or more multiple choice questions or other data entry options for the user. For example, if a user indicates at the first level that a treatment was skipped, PAEP system 100 may prompt a user to enter details on why the treatment was skipped at the second level. Once the second level of detail is entered, a user may be prompted to enter the third level of narrative tracking. Narrative tracking may include unstructured text or media entries, for example, freely typed narrative text and inclusion of photographs, videos, or other media. As an example, the AI component described herein may be trained by utilizing different stages of training sets in order to improve accuracy of the AI component (e.g., the different stages corresponding to the first level, the second level, and the third level described herein).

FIG. 129 shows another example of a taxonomy of data, showing a common language for reporting symptoms, behaviors, and treatment. FIG. 129 shows this example from a point of view or a patient or a family caregiver in a daily life setting. The common language may include one or more concepts as a concept set, and one or more phrases associated with a concept. There is a knowable set (e.g., stored in computer memory) of symptoms and behavior concepts (hereinafter, “observations”) that describe the possible physical and mental or emotion sensations that a user may notice and wish to track, to better describe and understand the user's current state of health.

Concepts may be assigned (e.g., by PAEP system 100) as primary title and tracking types, for example, TA, TB, TC, TD, where the title is a phrase that is used to identify the concept from primary search results in PAEP system 100 and may also be used for aggregation and reporting for research-related functions. The tracking type may be the default observation tracking type for a data capture, for example, TA may be tracked on a yes/no scale, TB may be tracked as an S-point-likened scale, and other tracking types may be used. To allow individual flexibility in use of tracking features of PAEP system 100 and to account for potential variability that is not yet fully known to researchers, multiple phrases and track types may be made available under one concept. See, for example, FIG. 130, showing an example implementation of such a taxonomy.

FIG. 130 illustrates dizziness being tracked as a concept, with room spinning, vertigo, and dizziness being tracked as yes or no phrases, and dizziness and vertigo further being tracked on a 0-5 severity scale. Depending on one or more factors, users may either be asked by PAEP system 100 to migrate tracking to a primary concept title and track-type, or the variations can be aggregated and converted to a common concept at the point of analysis, for example, when PAEP System 100 analyzes data from a plurality of users.

Development of the set of concepts may be completed iteratively through at least the following process shown in FIG. 131. First, one or more input data may be received or retrieved, including literature reviews, user reviews, and expert interviews. A first draft set (e.g., for training the AI component) may be created based on the one or more input data. The draft set may be used to open a tracking period. During the tracking period, PAEP system 100 may permit review of selected concepts, adding concepts, publication decisions and revision of the first draft set.

A second draft set and a suggested concepts draft set may be produced following the review, addition, and publication and revision of the first draft set. The second draft set and suggested concepts draft set may then be used as the first draft set, such that the second draft set and suggested concepts draft go through review of selected concepts, adding concepts, publication decisions and revision in an iterative process (e.g., further training of the AI component). This process (e.g., iterative training of the AI component) may be continued over time.

As one example, concept saturation may be achieved, which may occur when no new concepts are suggested by one or more parties, including but not limited to users and experts. The concept set may continue to be updated over time in response to changing human conditions, changes to a landscape of available treatments, or based on some other criteria.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A healthcare management computing system comprising at least one processor in communication with at least one memory, wherein the at least one processor is configured to:
receive a first input from a user computing device, the first input comprising health data associated with a first time period and a user profile associated with the user computing device;
identify historical health data stored in the at least one memory and associated with the health data, wherein the historical health data comprises first historical health data associated with the user profile before the first time period and second historical health data associated with a plurality of user profiles different from the user profile before the first time period;
based upon the health data and the historical health data, determine additional health data associated with the health data;
cause a plurality of data fields associated with the user profile to be populated with the additional health data;
receive a second input from the user computing device, the second input comprising modified additional health data comprising at least one modification to the additional health data; and
update the historical health data in the at least one memory to include the modified additional health data as being associated with the health data.

2. The healthcare management computing system of claim 1, wherein the at least one processor is further configured to:
receive a third input from the user computing device, the third input comprising second health data associated with a second time period and the user profile, wherein the second health data comprises at least one data entry that is the same as the health data;
identify the updated historical health data in the at least one memory; and
cause a second plurality of data fields associated with the user profile to be populated with the modified additional health data.

3. The healthcare management computing system of claim 1, wherein the first time period is one day.

4. The healthcare management computing system of claim 1, wherein the health data is associated with one of a typical day for a user associated with the user profile or an atypical day for the user.

5. The healthcare management computing system of claim 1, wherein the health data comprises at least one of an observation severity, a count, a presence, a numeric value with a unit, a tag, a timing, a duration, a free text note, a voice note, or a media image.

6. The healthcare management computing system of claim 1, wherein the at least one processor is further configured to:
generate a calendar associated with the user profile; and
cause an icon to be added to a portion of the calendar associated with the first time period, wherein the icon is associated with the health data.

7. The healthcare management computing system of claim 1, wherein the user profile and the plurality of user profiles different from the user profile are associated with a same health condition.

8. The healthcare management computing system of claim 1, wherein the at least one processor is further configured to, based upon the historical health data and the modified additional health data, determine one or more suggestions for a user associated with the user profile to address a health condition identified in the modified additional health data.

9. At least one non-transitory computer-readable storage medium with instructions stored thereon that, in response to execution by at least one processor, cause the at least one processor to:
receive a first input from a user computing device, the first input comprising health data associated with a first time period and a user profile associated with the user computing device;
identify historical health data stored in the at least one non-transitory computer-readable storage medium and associated with the health data, wherein the historical health data comprises first historical health data associated with the user profile before the first time period and second historical health data associated with a plurality of user profiles different from the user profile before the first time period;
based upon the health data and the historical health data, determine additional health data associated with the health data;
cause a plurality of data fields associated with the user profile to be populated with the additional health data;
receive a second input from the user computing device, the second input comprising modified additional health data comprising at least one modification to the additional health data; and
update the historical health data in the at least one non-transitory computer-readable storage medium to include the modified additional health data as being associated with the health data.

10. The non-transitory computer-readable storage medium of claim 9, wherein the at least one processor is further configured to:
receive a third input from the user computing device, the third input comprising second health data associated with a second time period and the user profile, wherein the second health data comprises at least one data entry that is the same as the health data;
identify the updated historical health data in the at least one non-transitory computer-readable storage medium; and
cause a second plurality of data fields associated with the user profile to be populated with the modified additional health data.

11. The non-transitory computer-readable storage medium of claim 9, wherein the first time period is one day.

12. The non-transitory computer-readable storage medium of claim 9, wherein the health data is associated with one of a typical day for a user associated with the user profile or an atypical day for the user.

13. The non-transitory computer-readable storage medium of claim 9, wherein the health data comprises at least one of an observation severity, a count, a presence, a numeric value with a unit, a tag, a timing, a duration, a free text note, a voice note, or a media image.

14. The non-transitory computer-readable storage medium of claim 9, wherein the at least one processor is further configured to:
generate a calendar associated with the user profile; and
cause an icon to be added to a portion of the calendar associated with the first time period, wherein the icon is associated with the health data.

15. The non-transitory computer-readable storage medium of claim 9, wherein the user profile and the plurality of user profiles different from the user profile are associated with a same health condition.

16. The non-transitory computer-readable storage medium of claim 9, wherein the at least one processor is further configured to, based upon the historical health data and the modified additional health data, determine one or more suggestions for a user associated with the user profile to address a health condition identified in the modified additional health data.

17. A method for healthcare management implemented by at least one processor in communication with at least one memory, the method comprising:
receiving a first input from a user computing device, the first input comprising health data associated with a first time period and a user profile associated with the user computing device;
identifying historical health data stored in the at least one memory and associated with the health data, wherein the historical health data comprises first historical health data associated with the user profile before the first time period and second historical health data associated with a plurality of user profiles different from the user profile before the first time period;
based upon the health data and the historical health data, determining additional health data associated with the health data;
causing a plurality of data fields associated with the user profile to be populated with the additional health data;
receiving a second input from the user computing device, the second input comprising modified additional health data comprising at least one modification to the additional health data; and
updating the historical health data in the at least one memory to include the modified additional health data as being associated with the health data.

18. The method of claim 17, further comprising:
receiving a third input from the user computing device, the third input comprising second health data associated with a second time period and the user profile, wherein the second health data comprises at least one data entry that is the same as the health data;
identifying the updated historical health data in the at least one memory; and
causing a second plurality of data fields associated with the user profile to be populated with the modified additional health data.

19. The method of claim 17, further comprising:
generating a calendar associated with the user profile; and
causing an icon to be added to a portion of the calendar associated with the first time period, wherein the icon is associated with the health data.

20. The method of claim 17, further comprising:
based upon the historical health data and the modified additional health data, determining one or more suggestions for a user associated with the user profile to address a health condition identified in the modified additional health data.
